(19) United States
(12) Patent Application Publication
(10) Pub. No.: US 2026/0274838 A1
(43) Pub. Date: Sep. 17, 2026

(54) 3H-PYRAZOLO[4.3:f] QUINOLINE-BASED COMPOUNDS AS STING ANTAGONISTS

(71) Applicant: PURDUE RESEARCH FOUNDATION, West Lafayette (IN)

(72) Inventors: Herman O. SINTIM, Westfield, IN (US); Neetu DAYAL, Agra (IN); Jones LAMPTEY, Kaneshie, Accra (GH); Wei Shiuan Wilson ONG, Singapore (SG)

(21) Appl. No.: 19/168,089

(22) PCT Filed: Mar. 22, 2024
(86) PCT No.: PCT/US2024/021183

§ 371 Date: Sep. 23, 2025

Related U.S. Application Data
(60) Provisional application No. 63/454,438, filed on Mar. 24, 2023.

(51) Int. Cl. C07D 471/04; A61K 31/4745; A61K 31/496; A61K 31/5377; A61K 31/541; C07D 471/14; C07D 491/147; C07D 491/20; C07D 495/14; C07D 519/00

(52) U.S. Cl. CPC C07D 471/04; A61K 31/4745; A61K 31/496; A61K 31/5377; A61K 31/541; C07D 471/14; C07D 491/147; C07D 491/20; C07D 495/14; C07D 519/00

(57) ABSTRACT

3H-pyrazolo[4,3-f]quinoline-based compounds that inhibit Stimulator of interferon genes (STING); compositions comprising same; and their use for treating STING-driven diseases such as STING-driven inflammatory diseases, autoimmune diseases, diabetes, cancer, traumatic brain injury, and fibrosis.

### CROSS-REFERENCE TO RELATED APPLICATION

This application claims priority to U.S. provisional patent application No. 63/454,438, which was filed Mar. 24, 2023, and which is hereby incorporated by reference in its entirety.

### TECHNICAL FIELD

The present disclosure relates to 3H-pyrazolo[4,3-f]quinoline-based compounds that are Stimulator of Interferon Genes (STING) inhibitors and their use in treating or inhibiting STING-driven diseases such as inflammatory diseases and response to injuries such as cardiac, traumatic, and brain injury.

### BACKGROUND

This section introduces aspects that may help facilitate a better understanding of the disclosure. Accordingly, these statements are to be read in this light and are not to be construed as admissions about what is or is not prior art.

The activation of innate immunity is critical for mounting swift responses toward adverse events such as pathogenic infections or cellular damage. The sensing of pathogen-associated molecular patterns (PAMPs) or damage-associated molecular patterns (DAMPs) by pattern recognition receptors allows for downstream activation and induction of pro-inflammatory cytokine expressions such as type 1 interferon, nuclear factor kappa-beta (NF-κβ) and other cytokines such as Interleukins 1 and 6, which play vital roles in T cell priming and activation. As a signaling motif indicating cellular damage or an invading DNA-containing pathogen, cytosolic double-stranded DNA is recognized by the DNA sensor cyclic GMP-AMP Synthase (cGAS)3, initiating 2′-3′ cyclic GMP-AMP (cGAMP) production through the catalysis of phosphodiester bond formation between adenosine triphosphate (ATP) and guanosine triphosphate (GTP). Binding of cGAMP to the universal cyclic dinucleotide sensor protein, Stimulator of IFN genes (STING), results in the recruitment of Tank Binding Kinase I (TBK1), which activates the transcription factor interferon regulatory factor 3 (IRF3) via phosphorylation. Following IRF3 activation and translocation to the nucleus, IRF3 serves as a transcriptional activator towards the potent induction of type 1 interferons, which are critical towards mounting appropriate immune responses against pathogenic invasion, as illustrated in FIG. 1.

The cGAS-STING axis plays an important role in protecting higher organisms against invading pathogens or cancer by promoting the production of cytokines and interferons. Despite the good side of the cGAS-STING activation, aberrant activation and dysfunction of this axis leads to chronic upregulation of cytokine expression, which has been shown to play a critical role in the development of chronic autoimmune disorders. Constitutive activation of STING due to gain of function (GOF) mutations plays a critical role in the development of debilitating diseases such as STING-associated vasculopathy with onset in infancy (SAVI), characterized by symptoms such as prominent vascular lesions and pulmonary inflammation. Activated STING is believed to play an important role in worsening various diseased states, such as traumatic brain injury, diabetic kidney disease, and colitis.

Alternatively, dysfunctions in cytosolic nucleic acid clearance mechanisms, as in the case of TREX1 exonuclease loss of function leading to DNA accumulation and chronic cGAS-STING activation, play major roles in the onset and development of other autoimmune disorders such as Aicardi-Goutières syndrome (AGS). With further mounting evidence suggesting cGAS-STING axis implication in various diseases where inflammation contributes towards disease onset, STING has since been viewed as an attractive target towards the amelioration of associated symptoms correlated with these autoimmune disorders and garnered significant attention (European Journal Medicinal Chemistry, 2019, 182, 111591; Frontiers in Immunology, 2022, 13, 5232). Antagonists of STING could play important roles in managing various inflammatory diseases. Agonists of STING have the potential to be used as immunotherapy.

To date, a small handful of small molecule-based inhibitors of STING have been discovered, primarily based on the use of cell-based quantification of cytokines downstream of the cGAS-STING pathway, such as interferon-β. This is achieved either through reporter cell bioassays or fundamental strategies such as the quantification of cytokine mRNA expression via quantitative polymerase chain reaction (qPCR) analyses, as with the discovery of previous STING antagonists such as C-176, H-151, and Astin C (see FIG. 2) (Nature 2018, 559, 269-273; Cell Rep 2018, 25, 3405-3421; Proc Natl Acad Sci USA 2021; 118, 24; ACS Med Chem Lett 2019; 10:92-97).

Therefore, there is a need to develop a compound that potently binds and inhibits STING. It is an object of the present disclosure to provide such a compound. This and other objects and advantages, as well as inventive features, will be apparent from the detailed description.

### SUMMARY

Provided is a method for treating or inhibiting disease associated with overacting of Stimulator of Interferon Genes (STING) comprising administering to a patient a therapeutically effective amount of a compound of formula (I):

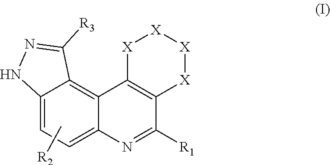

wherein
R₁ is selected from H, alkyl, cycloalkyl, heteroalkyl, cycloheteroalkyl, aryl, heteroaryl, alkenyl, alkynyl, and amide, wherein the amide is selected from sulfonamide, benzamide, nicotinamide, picolinamide, pyrimidine carboxamide, imidazole carboxamide, pyrazole carboxamide and a derivative of any of the forgoing;
R₂ is selected from H, alkyl, heteroalkyl, aryl, heteroaryl, alkenyl, alkynyl, and halogen;
R₃ is selected from H, alkyl, heteroalkyl, aryl, heteroaryl, alkenyl, and alkynyl; and each X is independently selected from O, S, SO, SO₂, CRR′, CNRR′, COR′, NR′ and Xₙ, wherein n is 0-2; wherein each R and R′ is independently selected from H, halogen, CF₃, CN, alkyl, aryl, heteroalkyl, heteroaryl, CONR₂R₂, COR₂, SO₂R₂, (C═O)NR₂, (C═NH)R₂, (C═O)OR₂, C₆H₅R₂, morpholine and piperazine, wherein R₂ is as defined above; or
R and R′, together with the atom to which they are attached, form a 4- to 6-membered cyclic or heterocyclic ring;
or a pharmaceutically acceptable salt, hydrate, tautomer, or optical isomer thereof or a pharmaceutical composition comprising the compound and a pharmaceutically acceptable carrier, excipient, or diluent.

Examples of the diseases associated with overacting of STING include, but are not limited to, inflammatory diseases, autoimmune diseases, diabetes, cancer, traumatic brain injury, and fibrosis. In some embodiments, the diseases are inflammatory diseases.

Provided is a compound of formula (IA):

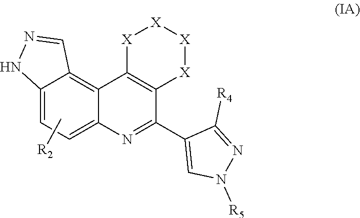

wherein
R₂ is selected from H, alkyl, heteroalkyl, aryl, heteroaryl, alkenyl, alkynyl, and halogen;
R₄ is selected from H, alkyl, cycloalkyl, heteroalkyl, heterocycloalkyl, aryl, heteroaryl, alkenyl, alkynyl, and amide, wherein the amide is selected from sulfonamide, benzamide, nicotinamide, picolinamide and a derivative of any of the forgoing; and R₄ is optionally substituted with OH, OR₂, halogen, CF₃, CN, NRR′, CONRR′, SO₂R₂, SO₂NRR′, NCOR₂ or NSO₂R₂, wherein each R and R′ is independently selected from H, halogen, CF₃, CN, alkyl, aryl, heteroalkyl, heteroaryl, CONR₂R₂, COR₂, SO₂R₂, (C═O)NR₂, (C═NH)R₂, (C═O)OR₂, C₆H₅R₂, morpholine and piperazine, wherein R₂ is as defined above; or
R and R′, together with the atom to which they are attached, form a 4- to 6-membered cyclic or heterocyclic ring;
R₅ is selected from alkyl, cycloalkyl, heteroalkyl, heterocycloalkyl, aryl, heteroaryl, alkenyl, alkynyl, and amide, wherein the amide is selected from sulfonamide, benzamide, nicotinamide, picolinamide and a derivative of any of the forgoing; and R₅ is optionally substituted with OH, OR₂, halogen, CF₃, CN, NRR′, CONRR′, SO₂R₂, SO₂NRR′, NCOR₂ or NSO₂R₂, wherein R₂, R and R′ are as defined above; and with the proviso that R₅ is not H, methyl, CF₃, CH₂—CH₂—OH, CH₂—CH₂—C₁, COOH, or optionally substituted phenyl, wherein the substitution on phenyl is selected from Ome, COOMe, and amino;
R₄ and R₅ can be the same or different; and
each X is independently selected from O, S, SO, SO₂, CRR′, CNRR′, COR′, NR′ and Xₙ, wherein n is 0-2; wherein each R and R′ is independently selected from H, halogen, CF₃, CN, alkyl, aryl, heteroalkyl, heteroaryl, CONR₂R₂, COR₂, SO₂R₂, (C═O)NR₂, (C═NH)R₂, (C═O)OR₂, C₆H₅R₂, morpholine and piperazine, wherein R₂ is as defined above; or
R and R′, together with the atom to which they are attached, form a 4- to 6-membered cyclic or heterocyclic ring;
or a pharmaceutically acceptable salt, hydrate, tautomer, or optical isomer thereof.

In some embodiments, the compound of formula (IA) is

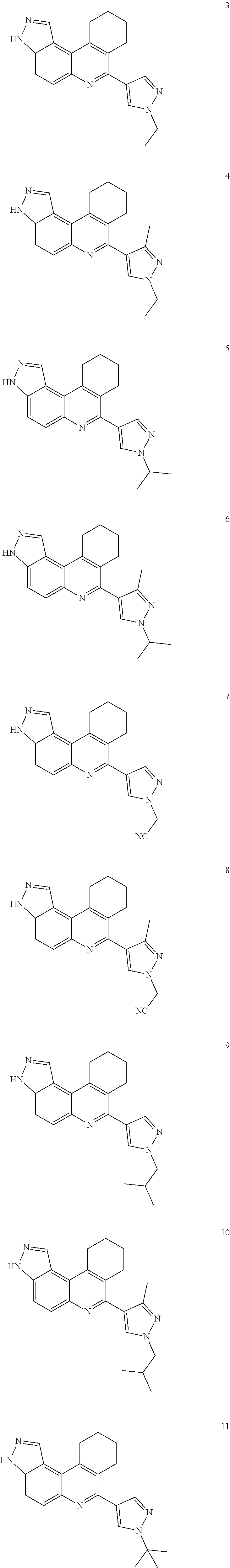

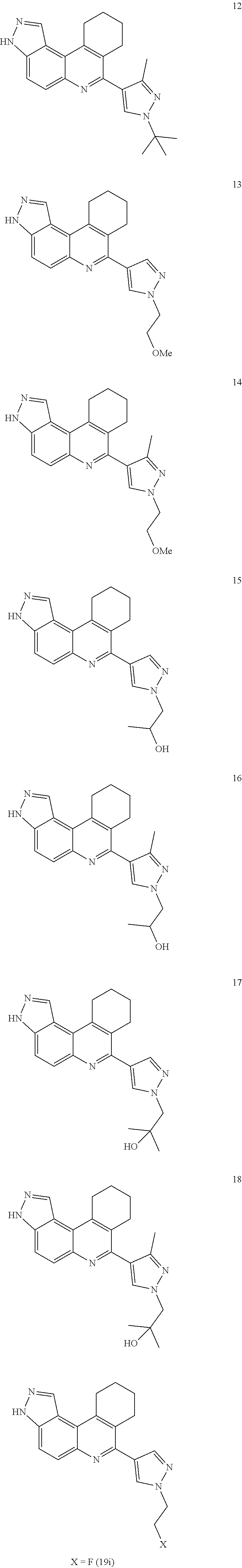

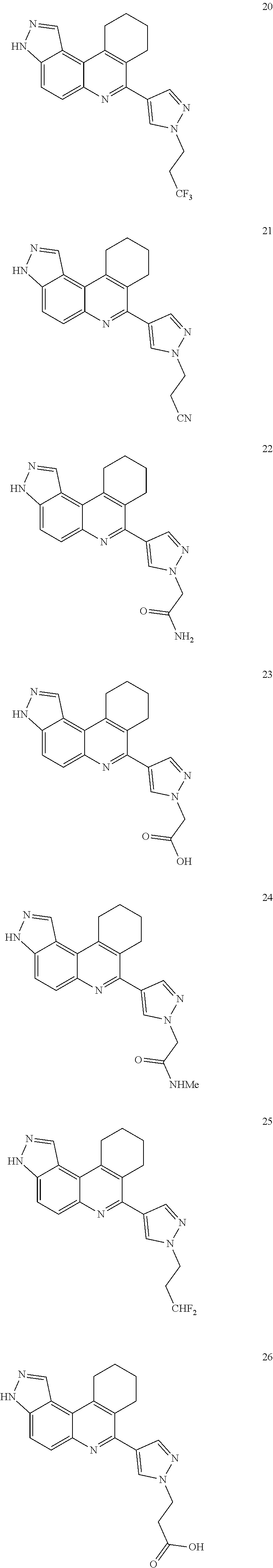

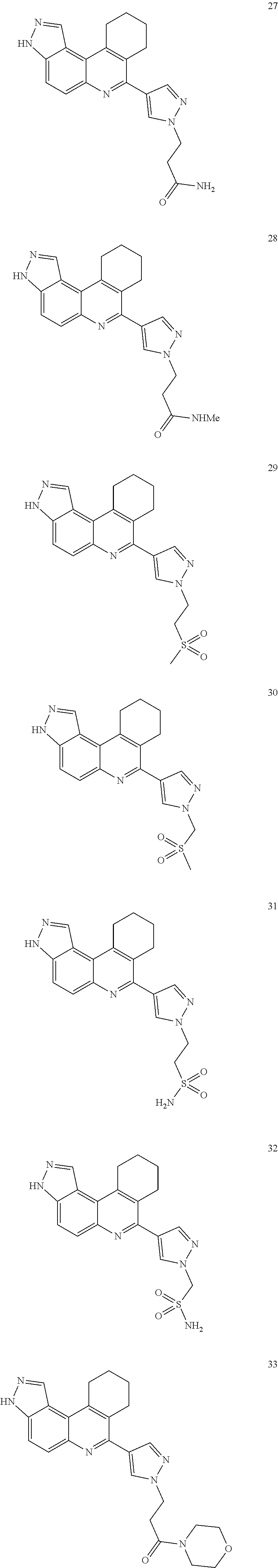

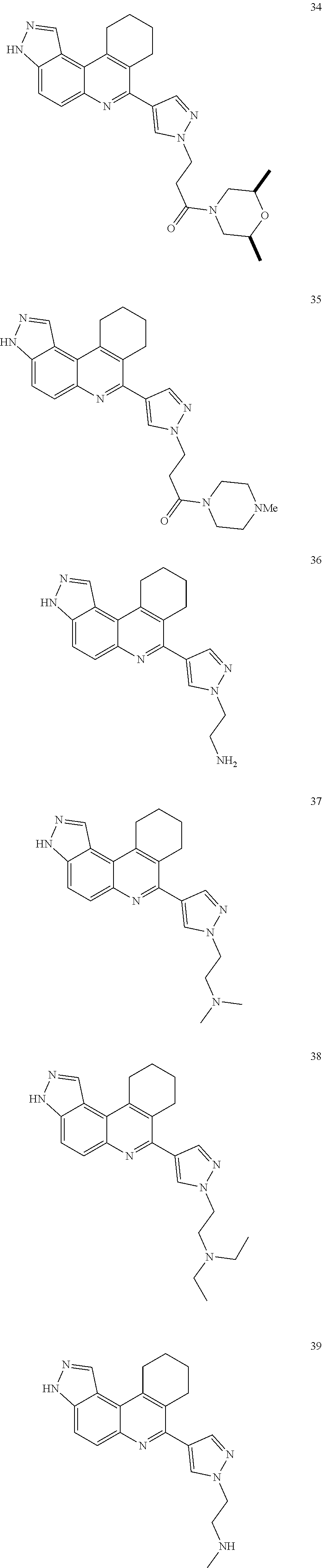 [SMILES: C[C@@H]1CN(C(=O)CCn2cc(-c3nc4ccc5[nH]ncc5c4c4c3CCCC4)cn2)C[C@H](C)O1 ; CN1CCN(C(=O)CCn2cc(-c3nc4ccc5[nH]ncc5c4c4c3CCCC4)cn2)CC1 ; NCCn1cc(-c2nc3ccc4[nH]ncc4c3c3c2CCCC3)cn1 ; CN(C)CCn1cc(-c2nc3ccc4[nH]ncc4c3c3c2CCCC3)cn1 ; CCN(CC)CCn1cc(-c2nc3ccc4[nH]ncc4c3c3c2CCCC3)cn1 ; CNCCn1cc(-c2nc3ccc4[nH]ncc4c3c3c2CCCC3)cn1]

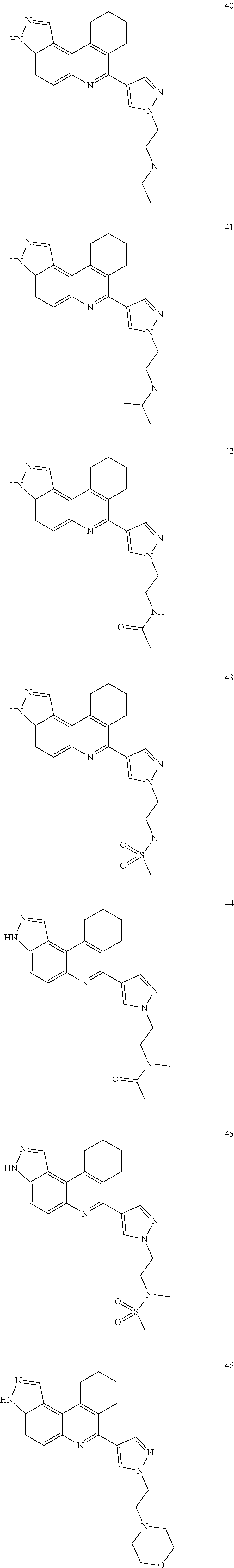

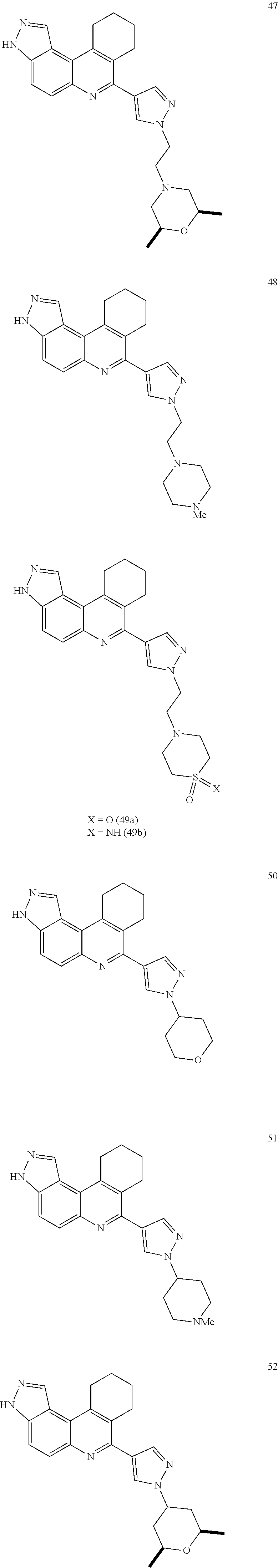

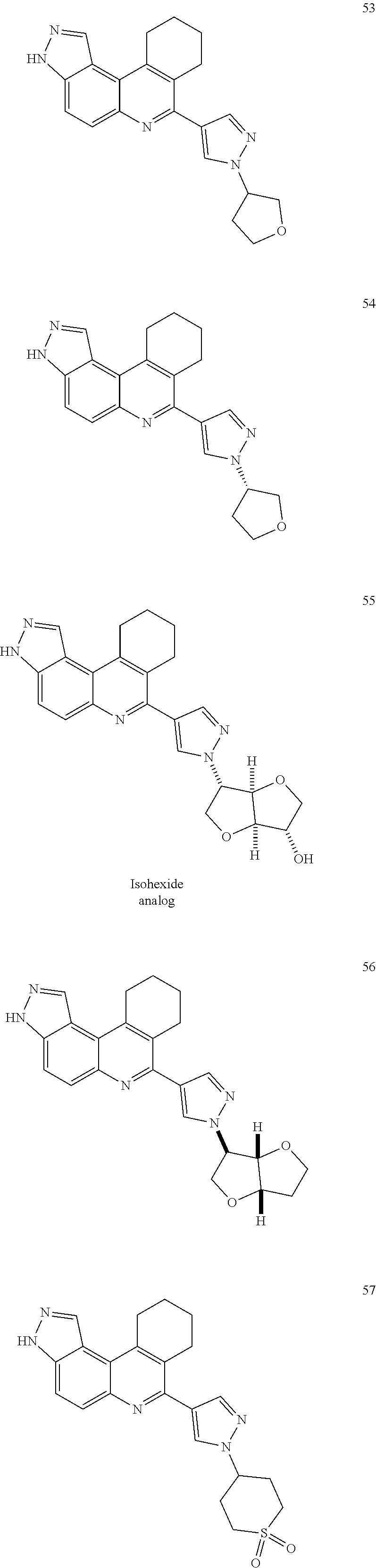

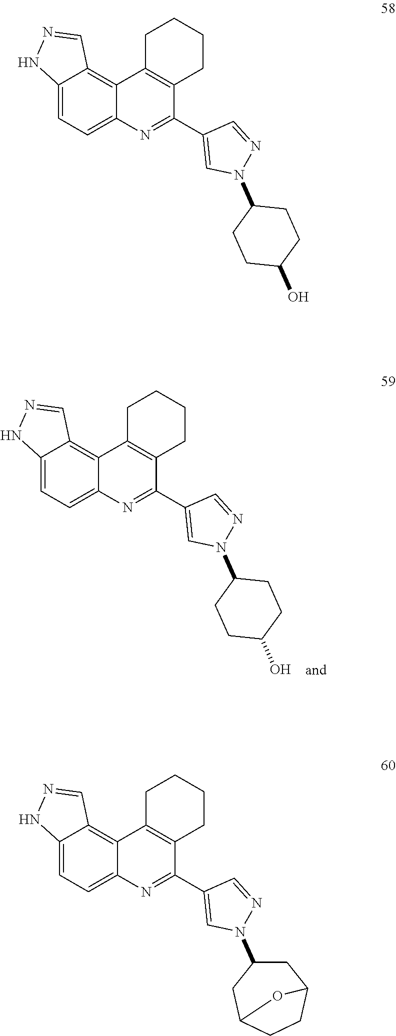

or a pharmaceutically acceptable salt, hydrate, tautomer, or optical isomer thereof.

In some embodiments, the compound of formula (IA) is

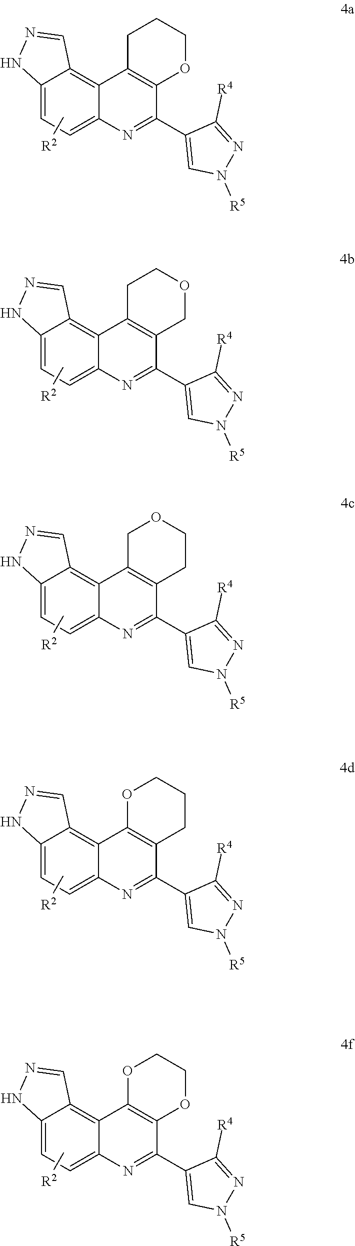

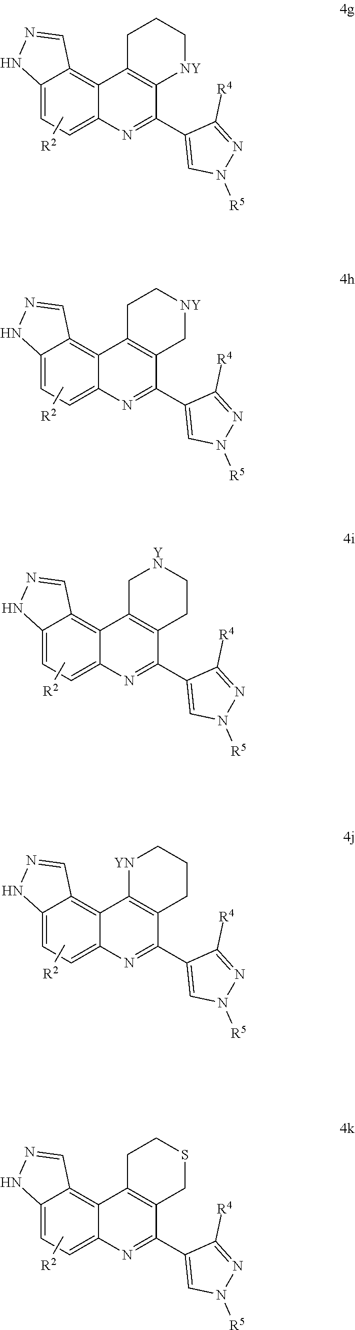

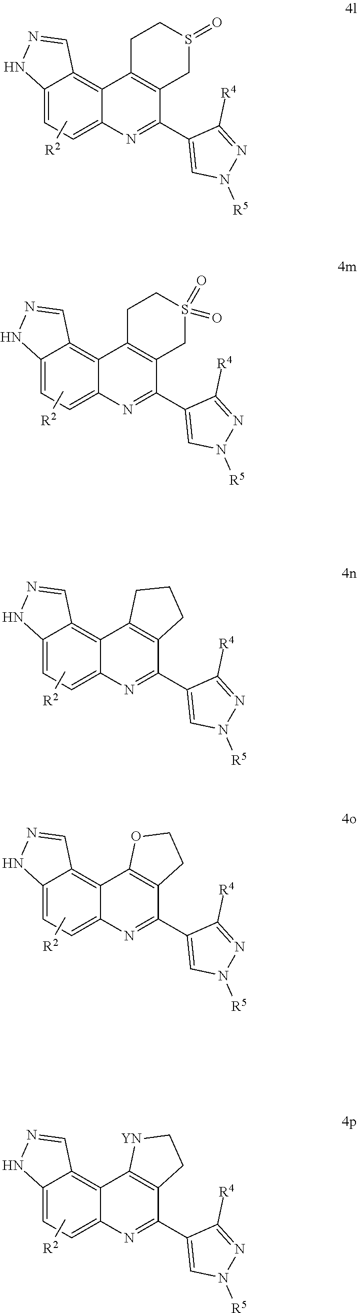

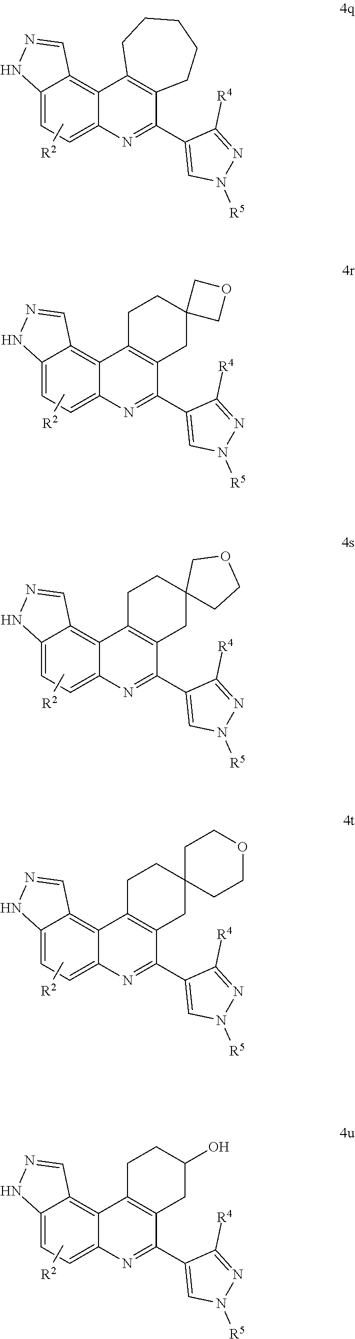

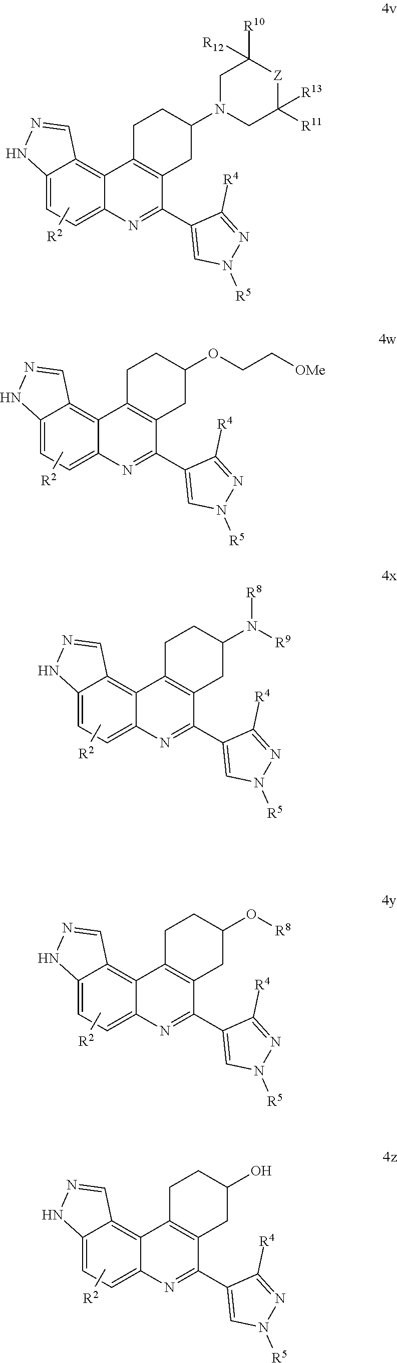

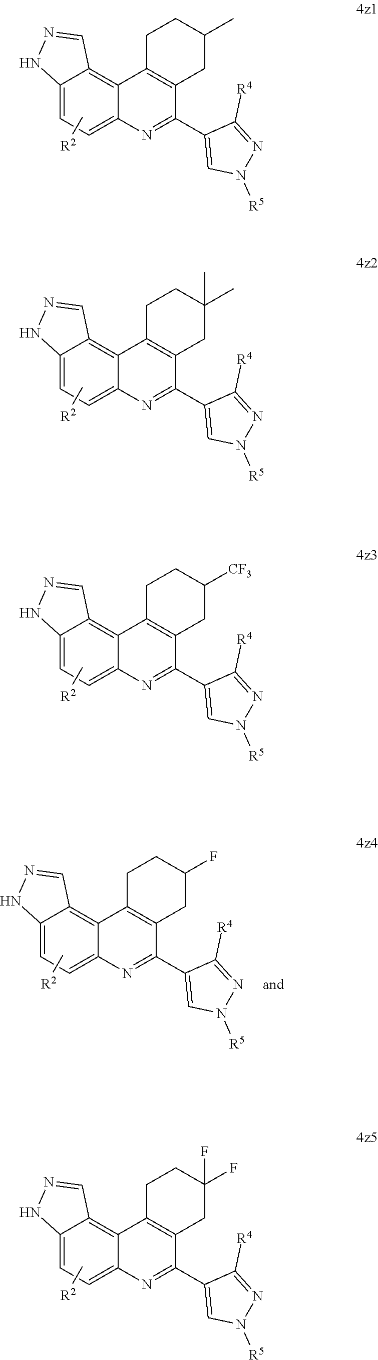

wherein
R₂ is selected from H, alkyl, heteroalkyl, aryl, heteroaryl, alkenyl, alkynyl, and halogen;
each R₄ and R₅ is independently selected from H, alkyl, cycloalkyl, heteroalkyl, heterocycloalkyl, aryl, heteroaryl, alkenyl, alkynyl, and amide, wherein the amide is selected from sulfonamide, benzamide, nicotinamide, picolinamide and a derivative of any of the forgoing, wherein alkyl is optionally substituted with OH, OR₂, halogen, CF₃, CN, NRR′, CONRR′, SO₂R₂, SO₂NRR′, NCOR₂ or NSO₂R₂, wherein R₂, R and R′ are as defined above;
each R₈ and R₉ is independently selected from H, methyl, alkyl, and heteroalkyl;
each R₁₀, R₁₁, R₁₂, and R₁₃ is independently selected from H, methyl, alkyl, and heteroalkyl; or R₁₀ and R₁₂, together with the atom to which they are attached, form a 4- to 6-membered cyclic or heterocyclic ring; or
R₁₁ and R₁₃, together with the atom to which they are attached, form a 4- to 6-membered cyclic or heterocyclic ring;
Z is selected from O, S, SO, SO₂, CRR′, CNRR′, COR′ and NR′, wherein each R and R′ is as defined above;
Y is selected from COR₂, SO₂R₂, (C═O)NR₂, (C═NH)R₂, (C═O)OR₂ and C₆H₅R₂, wherein R₂ is as defined above;
or a pharmaceutically acceptable salt, hydrate, tautomer, or optical isomer thereof.

Provided is a compound of formula (IB):

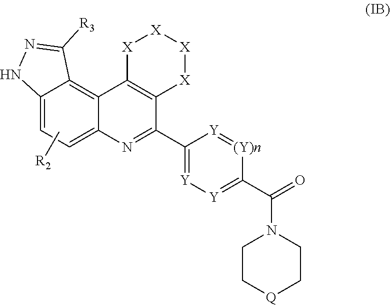

wherein
R₂ is selected from H, alkyl, heteroalkyl, aryl, heteroaryl, alkenyl, alkynyl, and halogen;
R₃ is selected from H, alkyl, heteroalkyl, aryl, heteroaryl, alkenyl, and alkynyl; each X is independently selected from O, S, SO, SO₂, CRR′, CNRR′, COR′, NR′ and Xₙ, wherein n is 0-2; wherein each R and R′ is independently selected from H, halogen, CF₃, CN, alkyl, aryl, heteroalkyl, heteroaryl, CONR₂R₂, COR₂, SO₂R₂, (C═O)NR₂, (C═NH)R₂, (C═O)OR₂, C₆H₅R₂, morpholine and piperazine, wherein R₂ is as defined above; or
R and R′, together with the atom to which they are attached, form a 4- to 6-membered cyclic or heterocyclic ring;
Y is N or CR₂, wherein R₂ is as defined above;
n is 0 or 1;
Q is selected from NR″, CR₂R₂, N(COR₂), N(SO₂)R₂, N(C═O)NR₂, N(C═NH)R₂, and N(C═O)OR₂, with the proviso that when Q is NR″, R″ is not H, methyl, isopropyl, CH₂CH₂OH, COCH₃ or SO₂-methyl; and when Q is CR₂R₂, R₂ is not H or dimethyl amine;
or a pharmaceutically acceptable salt, hydrate, tautomer, or optical isomer thereof.

In some embodiments, the compound of formula (IB) is

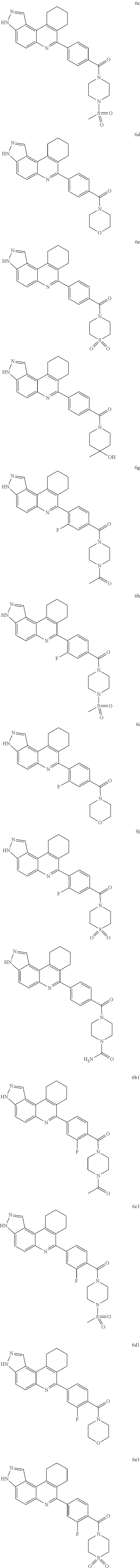

[chemical structure]

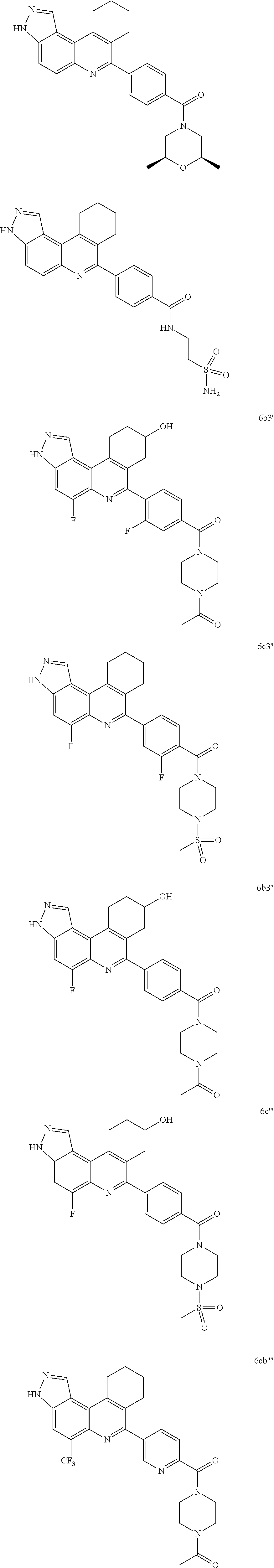

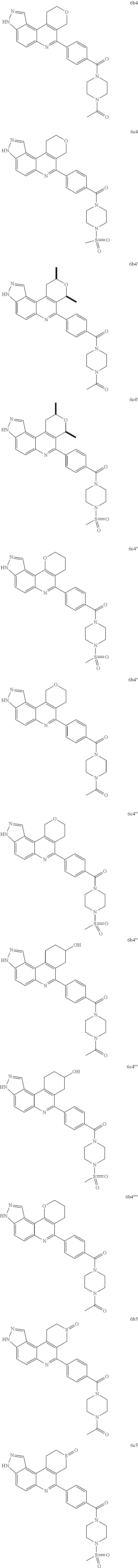

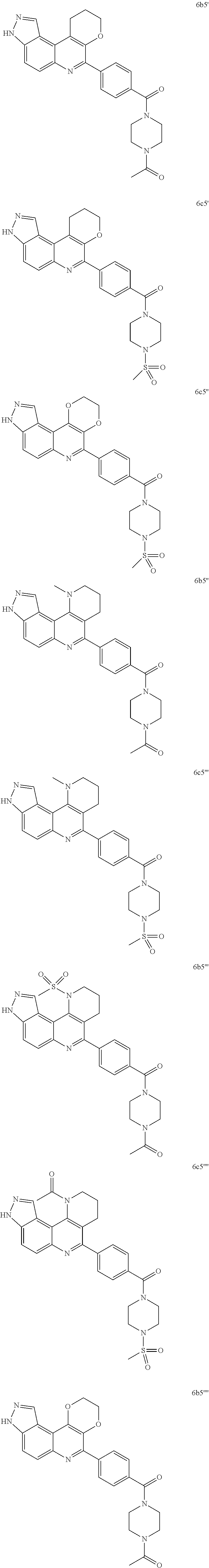

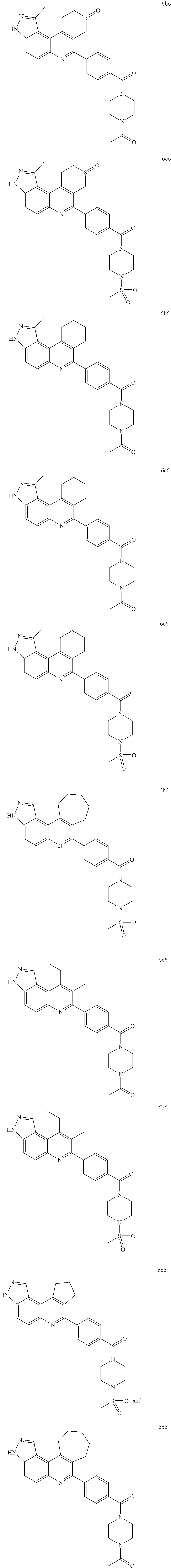

or a pharmaceutically acceptable salt, hydrate, tautomer, or optical isomer thereof.

In some embodiments, the compound of formula (I) is represented by structures:

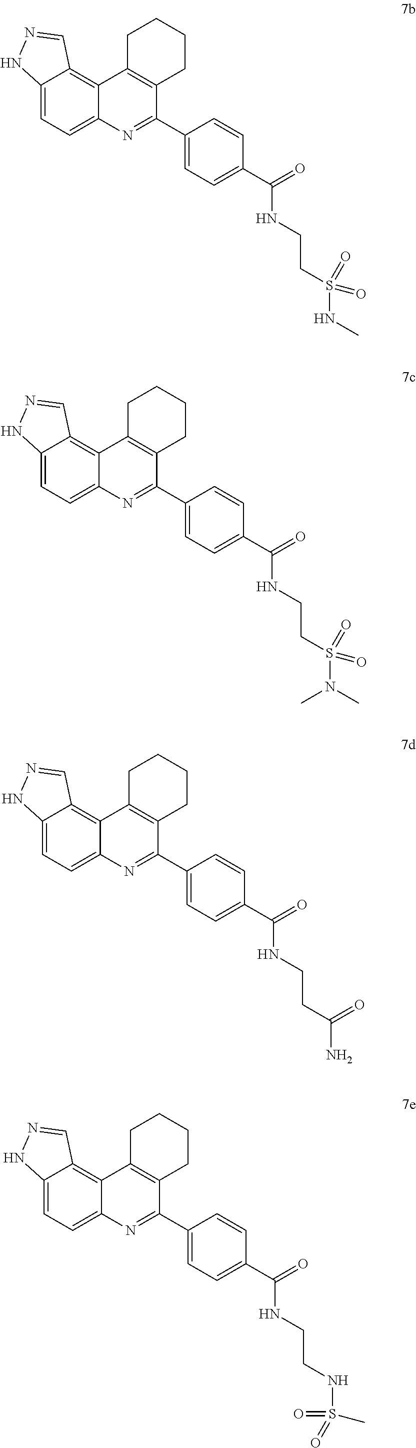 [SMILES: CNS(=O)(=O)CCNC(=O)c1ccc(-c2nc3ccc4[nH]ncc4c3c3c2CCCC3)cc1 ; CN(C)S(=O)(=O)CCNC(=O)c1ccc(-c2nc3ccc4[nH]ncc4c3c3c2CCCC3)cc1 ; NC(=O)CCNC(=O)c1ccc(-c2nc3ccc4[nH]ncc4c3c3c2CCCC3)cc1 ; CS(=O)(=O)NCCNC(=O)c1ccc(-c2nc3ccc4[nH]ncc4c3c3c2CCCC3)cc1]

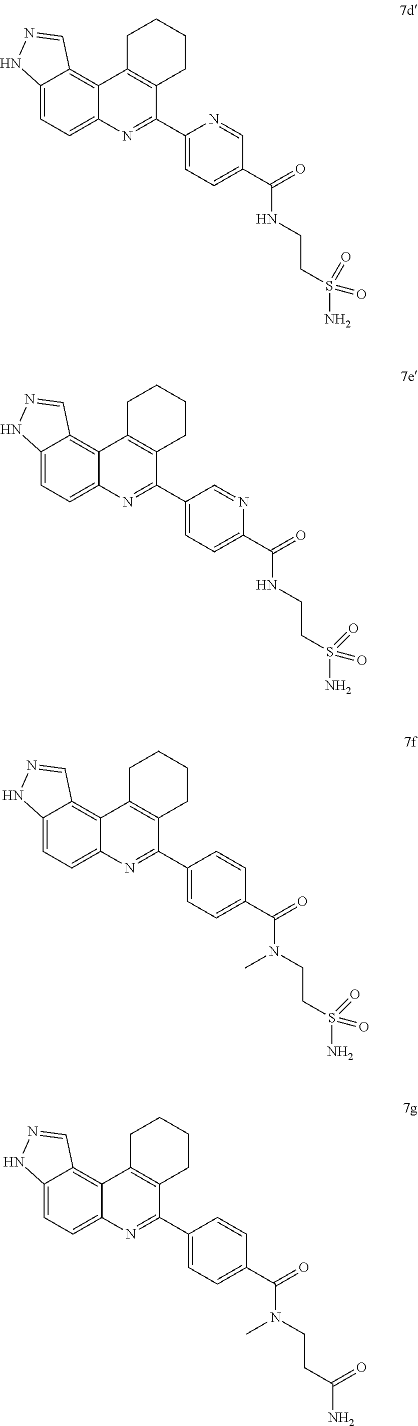 [SMILES: NS(=O)(=O)CCNC(=O)c1ccc(-c2nc3ccc4[nH]ncc4c3c3c2CCCC3)nc1 ; NS(=O)(=O)CCNC(=O)c1ccc(-c2nc3ccc4[nH]ncc4c3c3c2CCCC3)cn1 ; CN(CCS(N)(=O)=O)C(=O)c1ccc(-c2nc3ccc4[nH]ncc4c3c3c2CCCC3)cc1 ; CN(CCC(N)=O)C(=O)c1ccc(-c2nc3ccc4[nH]ncc4c3c3c2CCCC3)cc1]

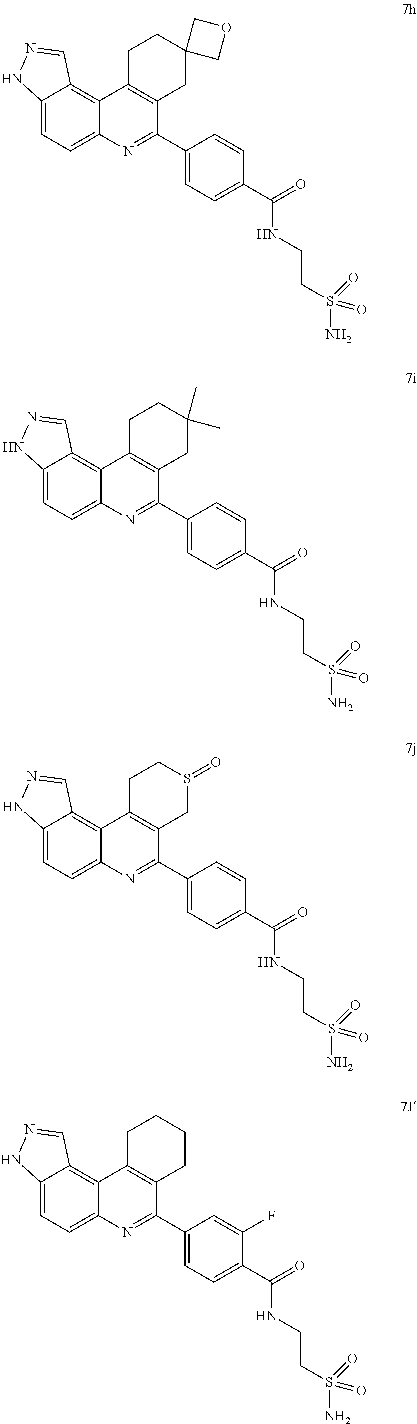 [SMILES: NS(=O)(=O)CCNC(=O)c1ccc(-c2nc3ccc4[nH]ncc4c3c3c2CC2(CC3)COC2)cc1 ; CC1(C)CCc2c(c(-c3ccc(C(=O)NCCS(N)(=O)=O)cc3)nc3ccc4[nH]ncc4c23)C1 ; NS(=O)(=O)CCNC(=O)c1ccc(-c2nc3ccc4[nH]ncc4c3c3c2CS(=O)CC3)cc1 ; NS(=O)(=O)CCNC(=O)c1ccc(-c2nc3ccc4[nH]ncc4c3c3c2CCCC3)cc1F]

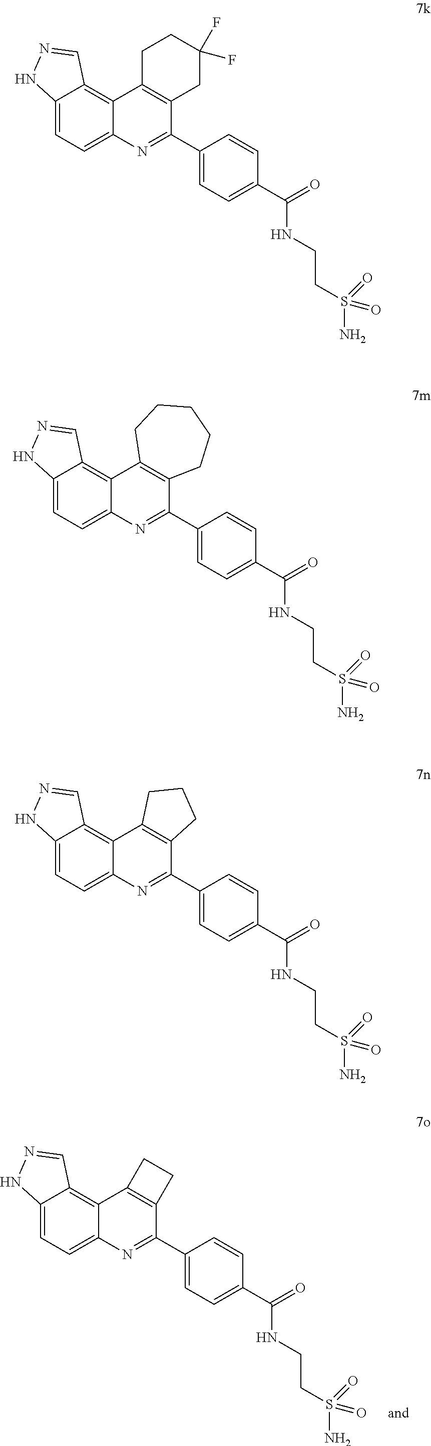 [SMILES: NS(=O)(=O)CCNC(=O)c1ccc(-c2nc3ccc4[nH]ncc4c3c3c2CC(F)(F)CC3)cc1 ; NS(=O)(=O)CCNC(=O)c1ccc(-c2nc3ccc4[nH]ncc4c3c3c2CCCCC3)cc1 ; NS(=O)(=O)CCNC(=O)c1ccc(-c2nc3ccc4[nH]ncc4c3c3c2CCC3)cc1 ; NS(=O)(=O)CCNC(=O)c1ccc(-c2nc3ccc4[nH]ncc4c3c3c2CC3)cc1]

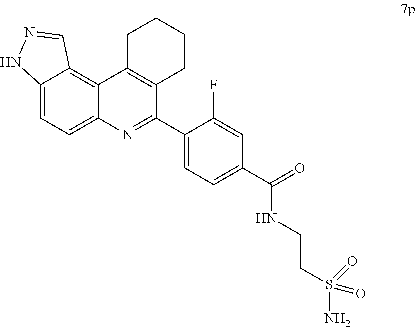 [SMILES: NS(=O)(=O)CCNC(=O)c1ccc(-c2nc3ccc4[nH]ncc4c3c3c2CCCC3)c(F)c1]

or a pharmaceutically acceptable salt, hydrate, tautomer, or optical isomer thereof.

Provided is a pharmaceutical composition comprising a compound of formula (I) or a pharmaceutically acceptable salt, hydrate, tautomer, or optical isomer thereof and a pharmaceutically acceptable carrier, excipient, or diluent.

Provided is a pharmaceutical composition comprising a compound of formula (IA) or (IB) or a pharmaceutically acceptable salt, hydrate, tautomer, or optical isomer of either of the foregoing, and a pharmaceutically acceptable carrier, excipient, or diluent.

Further provided is a method of treating or inhibiting disease associated with overacting of Stimulator of Interferon Genes (STING) comprising administering to a patient a therapeutically effective amount of a compound of formula (IA) or a pharmaceutically acceptable salt, hydrate, tautomer, or optical isomer thereof or a pharmaceutical composition comprising the compound and a pharmaceutically acceptable carrier, excipient, or diluent.

Still further provided is a method of treating or inhibiting disease associated with overacting of Stimulator of Interferon Genes (STING) comprising administering to a patient a therapeutically effective amount of a compound of formula (IB) or a pharmaceutically acceptable salt, hydrate, tautomer, or optical isomer thereof or a pharmaceutical composition comprising the compound and a pharmaceutically acceptable carrier, excipient, or diluent.

Examples of the diseases associated with overacting of STING include, but are not limited to inflammatory diseases, autoimmune diseases, diabetes, cancer, traumatic brain injury, and fibrosis. In some embodiments, the diseases are inflammatory diseases.

### BRIEF DESCRIPTION OF THE DRAWINGS

The above and other objects, features, and advantages of the present disclosure will be apparent when the description is read in conjunction with the drawings.

FIG. 1 shows the cyclic GMP-AMP Synthase-Stimulator of Interferon Genes (cGAS-STING) activation pathway.

FIG. 2 shows the structures of reported small compounds, e.g., H-151, C-178, Astin-C, SN-011, and C-18, that act as STING antagonists.

FIG. 3 shows probe displacement by STING binder. A) illustrates the structure of the F-c-di-GMP probe. B) illustrates a schematic diagram depicting the repurposing of the developed FP assay for the potential screening of small molecule binders of STING, which competitively displace F-c-di-GMP.

FIG. 4A shows the synthesis of quinoline compounds using the Doebner-Povarov multicomponent reaction and structure of a compound HSD1077.

FIG. 4 B shows the plot depicting anisotropy values versus the concentration of the compound HSD1077, which indicates dose-dependent competitive probe displacement. Concentrations used for probe and STING were 50 nM and 10 μM, respectively.

FIG. 5A shows analogues modified at ring A tested for STING binding through STING-FP assay.

FIG. 5 B shows the fraction of probe bound to STING (Fbound) upon incubation with listed drug compounds. Fluorophore-c-di-GMP was used at a concentration of 50 nM, STING at 10 μM. All compounds, for example, ADU-S100, DIABZI-3, and compound HSD1077, were used at 20 μM concentrations.

FIG. 6A shows analogues modified at cyclohexyl ring B tested for STING binding through STING-FP assay.

FIG. 6 B shows the fraction of probe bound to STING (Fbound) upon incubation with drug compounds, for example, ADU-S100, DIABZI-3, and compound HSD1077.

FIG. 7A shows analogues modified at ring C tested for STING binding through STING-FP assay.

FIG. 7 B shows the fraction of probe bound to STING (Fbound) upon incubation with drug compounds, for example, ADU-S100, DIABZI-3, and compound HSD1077. Fluorophore-c-di-GMP was used at a concentration of 50 nM, STING at 10 μM. The compounds ADU-S100, DIABZI-3, and compound HSD1077 were used at 20 μM concentrations.

FIG. 8A shows the treatment of compound HSD1077 to RAW Interferon-stimulated gene (ISG) Blue for 24 hours showed non-significant changes in interferon expression levels. Pre-treatment of compound HSD1077, followed by cGAMP induction, results in attenuated expression of type 1 interferon in a dose-dependent manner.

FIG. 8B illustrates RAW ISG cells were pre-treated with either compound HSD1077 or compound H-151 as a positive control for 6 hours and subsequently stimulated with 100 μM of cGAMP for 3 hours for induction of Interferon-β. mRNA levels were quantified by RT-PCR. Gene expression was normalized with β-actin. Experiments were performed in two biological replicates. Error bars indicate the error of the mean of two independent experiments.

FIG. 9 shows compound HSD1077 attenuates STING and IRF3 phosphorylation in Raw ISG cells. Raw ISG cells were treated with 5 μM compound HSD1077 or 1 μM compound H₁₅₁ for 4 hours, followed by 100 μM cGAMP treatment for 3 hours. Control cells were treated with dimethyl sulfoxide (DMSO) and sterile water. p-STING and p-IRF3 levels were analyzed using western blotting. The experiment was done in two biological replicates.

FIG. 10A shows pre-treatment of compound HSD1077 to THP-1 dual monocytes, human cells for 2 hours, prior to induction of interferon by 2′-3′ cGAMP stimulation for 24 hours, results in attenuated expression of interferon in a dose-dependent manner.

FIG. 10 B shows the treatment of compound HSD1077 to human THP-1 dual (KI STING N154S) cells with a point mutation resulting in a gain of function showed a decrease in interferon expression levels upon 24 hours of incubation.

FIG. 11 shows the treatment of compounds to THP-1 Dual (KI STING N154S) cells with a point mutation resulting in a gain of function, indicating a decrease in interferon expression levels upon 24 hours of incubation.

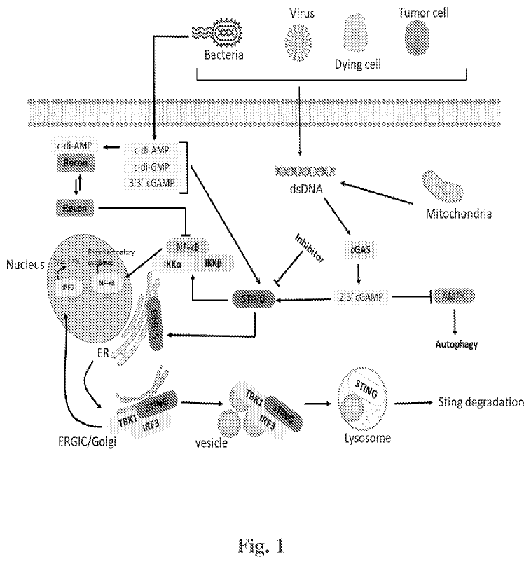

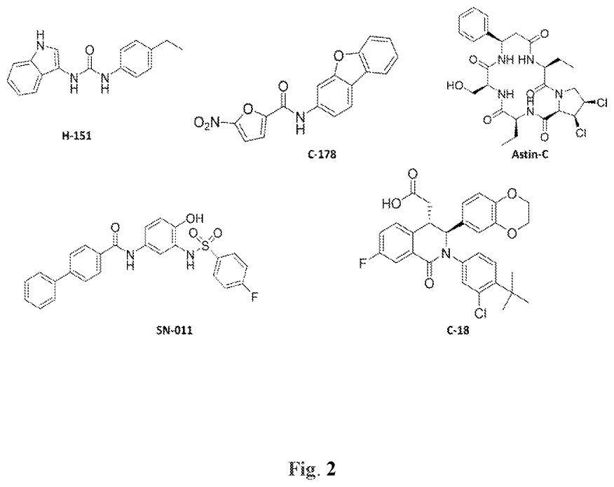

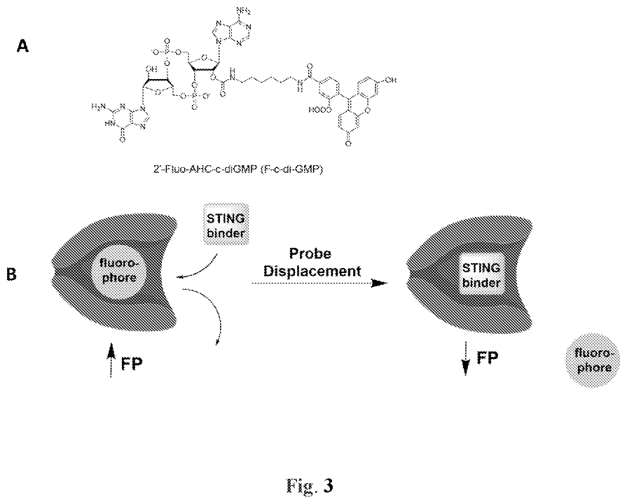

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended.

The term ‘STING’ (also known as MITA, MPYS, ERIS, and TMEM173) refers to Stimulator of Interferon Genes or Stimulator of IFN genes, an adaptor protein that is functionally activated by cyclic dinucleotides, which leads to the production of interferons and inflammatory cytokines such as TNF, IL-1, IL-6, IFNγ, type 1 interferon (IFN) and, nuclear factor kappa-beta (NF-κβ).

The present disclosure is predicated, at least in part, on the discovery that the 3H-pyrazolo[4,3-f]quinoline moiety is a privileged moiety that binds to hinge regions in kinases (Int′. Pat. Appl. No. WO2018183586A1, U. S. Pat. Pub. No. 20200308173A1, U.S. patent Ser. No. 11/040,973); substitution of the 3H-pyrazolo[4,3-f]quinoline moiety leads to compounds that inhibit kinases with antiproliferative properties. For many therapeutic applications, compounds of the 3H-pyrazolo[4,3-f]quinoline class can have low cytotoxicity against mammalian cells while displaying high potency against receptors that regulate diseased states.

In view of the above, the present disclosure provides compounds that comprise a 3H-pyrazolo[4,3-f]quinoline moiety that binds to STING to inhibit STING's function and can be tolerable to many mammalian cell lines at concentrations of 0.5 μM or higher.

Provided is a method of treating or inhibiting disease associated with overacting of Stimulator of Interferon Genes (STING) comprising administering to a patient a therapeutically effective amount of a compound of formula (I):

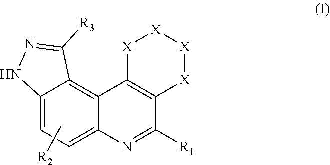

wherein
R₁ is selected from H, alkyl, cycloalkyl, heteroalkyl, cycloheteroalkyl, aryl, heteroaryl, alkenyl, alkynyl, and amide, wherein the amide is selected from sulfonamide, benzamide, nicotinamide, picolinamide, pyrimidine carboxamide, imidazole carboxamide, pyrazole carboxamide and a derivative of any of the foregoing;
R₂ is selected from H, alkyl, heteroalkyl, aryl, heteroaryl, alkenyl, alkynyl, and halogen;
R₃ is selected from H, alkyl, heteroalkyl, aryl, heteroaryl, alkenyl, and alkynyl; and each X is independently selected from O, S, SO, SO₂, CRR′, CNRR′, COR′, NR′ and Xₙ, wherein n is 0-2; wherein each R and R′ is independently selected from H, halogen, CF₃, CN, alkyl, aryl, heteroalkyl, heteroaryl, CONR₂R₂, COR₂, SO₂R₂, (C═O)NR₂, (C═NH)R₂, (C═O)OR₂, C₆H₅R₂, morpholine and piperazine, wherein R₂ is as defined above; or
R and R′, together with the atom to which they are attached, form a 4- to 6-membered cyclic or heterocyclic ring;
or a pharmaceutically acceptable salt, hydrate, tautomer, or optical isomer thereof or a pharmaceutical composition comprising the compound and a pharmaceutically acceptable carrier, excipient, or diluent.

In some embodiments, the compound of formula (I) is represented by a compound of formula (IA):

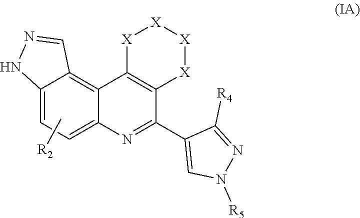

wherein
R₂ is selected from H, alkyl, heteroalkyl, aryl, heteroaryl, alkenyl, alkynyl, and halogen;
R₄ is selected from H, alkyl, cycloalkyl, heteroalkyl, heterocycloalkyl, aryl, heteroaryl, alkenyl, alkynyl, and amide, wherein the amide is selected from sulfonamide, benzamide, nicotinamide, picolinamide and a derivative of any of the forgoing; and R₄ is optionally substituted with OH, OR₂, halogen, CF₃, CN, NRR′, CONRR′, SO₂R₂, SO₂NRR′, NCOR₂ or NSO₂R₂, wherein each R and R′ is independently selected from H, halogen, CF₃, CN, alkyl, aryl, heteroalkyl, heteroaryl, CONR₂R₂, COR₂, SO₂R₂, (C═O)NR₂, (C═NH)R₂, (C═O)OR₂, C₆H₅R₂, morpholine and piperazine, wherein R₂ is as defined above; or
R and R′, together with the atom to which they are attached, form a 4- to 6-membered cyclic or heterocyclic ring;
R₅ is selected from alkyl, cycloalkyl, heteroalkyl, heterocycloalkyl, aryl, heteroaryl, alkenyl, alkynyl, and amide, wherein the amide is selected from sulfonamide, benzamide, nicotinamide, picolinamide and a derivative of any of the forgoing, wherein R₅ is optionally substituted with OH, OR₂, halogen, CF₃, CN, NRR′, CONRR′, SO₂R₂, SO₂NRR′, NCOR₂ or NSO₂R₂, wherein
R₂, R and R′ are as defined above; and with the proviso that R₅ is not H, methyl, CF₃, CH₂—CH₂—OH, CH₂—CH₂—C₁, COOH, or optionally substituted phenyl, wherein the substitution on phenyl is selected from OMe, COOMe, and amino;
R₄ and R₅ can be the same or different; and
each X is independently selected from O, S, SO, SO₂, CRR′, CNRR′, COR′, NR′ and Xₙ, wherein n is 0-2; wherein each R and R′ is independently selected from H, halogen, CF₃, CN, alkyl, aryl, heteroalkyl, heteroaryl, CONR₂R₂, COR₂, SO₂R₂, (C═O)NR₂, (C═NH)R₂, (C═O)OR₂, C₆H₅R₂, morpholine and piperazine, wherein R₂ is as defined above; or
R and R′, together with the atom to which they are attached, form a 4- to 6-membered cyclic or heterocyclic ring;
or a pharmaceutically acceptable salt, hydrate, tautomer, or optical isomer thereof.

In some embodiments, the compound of formula (IA) is

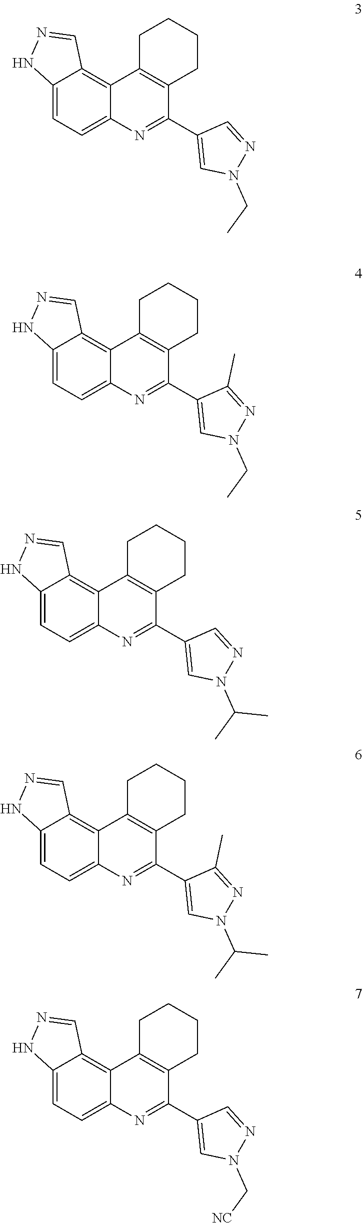

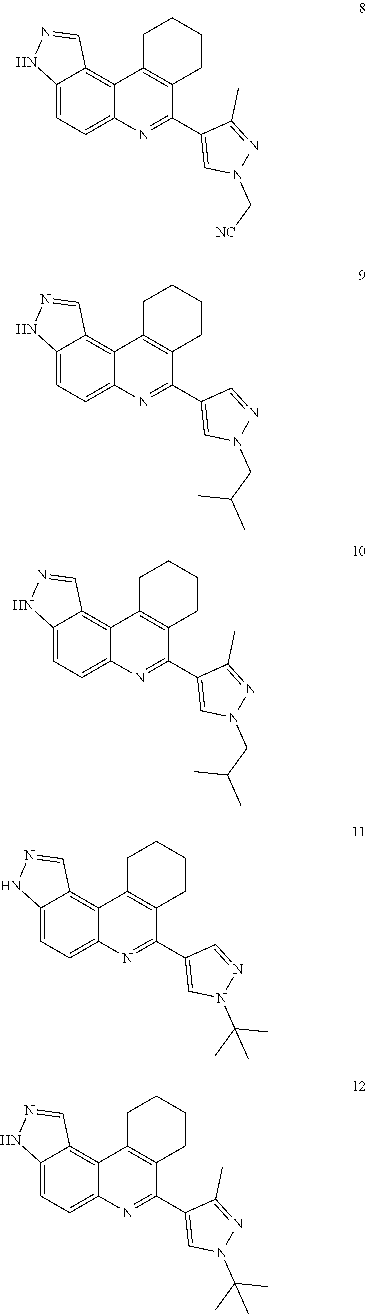 [SMILES: Cc1nn(CC#N)cc1-c1nc2ccc3[nH]ncc3c2c2c1CCCC2 ; CC(C)Cn1cc(-c2nc3ccc4[nH]ncc4c3c3c2CCCC3)cn1 ; Cc1nn(CC(C)C)cc1-c1nc2ccc3[nH]ncc3c2c2c1CCCC2 ; CC(C)(C)n1cc(-c2nc3ccc4[nH]ncc4c3c3c2CCCC3)cn1 ; Cc1nn(C(C)(C)C)cc1-c1nc2ccc3[nH]ncc3c2c2c1CCCC2]

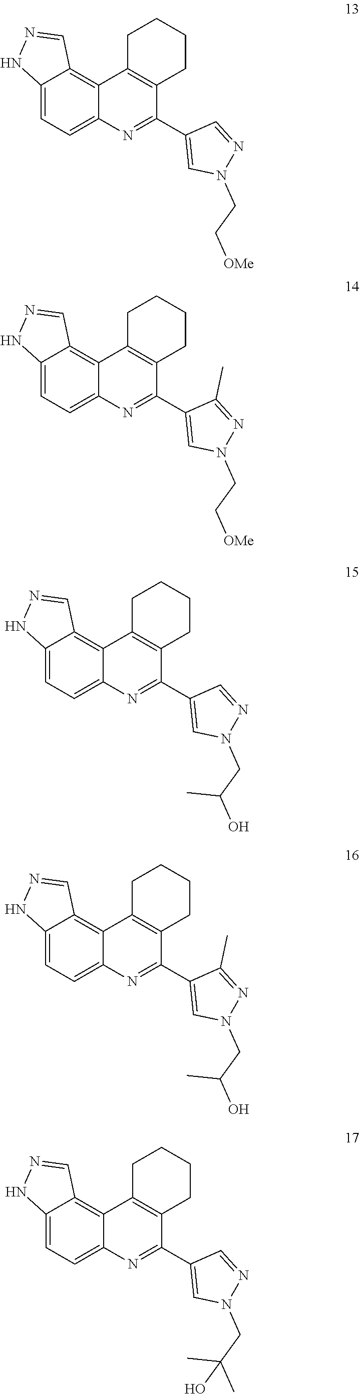 [SMILES: COCCn1cc(-c2nc3ccc4[nH]ncc4c3c3c2CCCC3)cn1 ; COCCn1cc(-c2nc3ccc4[nH]ncc4c3c3c2CCCC3)c(C)n1 ; CC(O)Cn1cc(-c2nc3ccc4[nH]ncc4c3c3c2CCCC3)cn1 ; Cc1nn(CC(C)O)cc1-c1nc2ccc3[nH]ncc3c2c2c1CCCC2 ; CC(C)(O)Cn1cc(-c2nc3ccc4[nH]ncc4c3c3c2CCCC3)cn1]

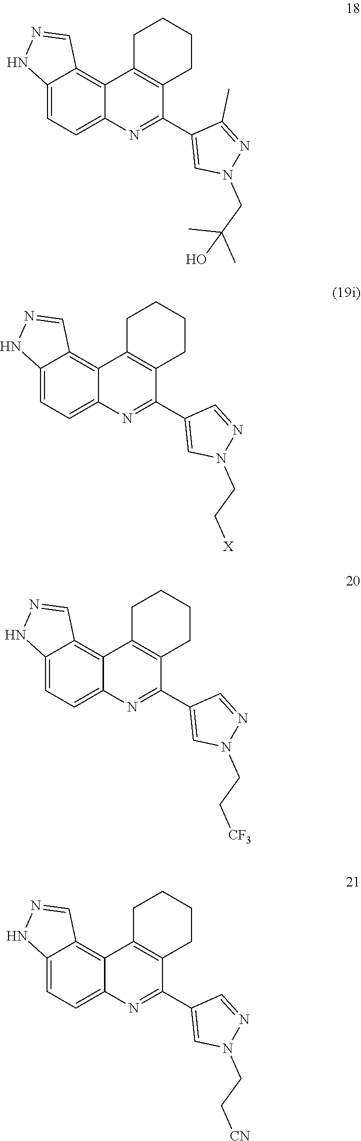

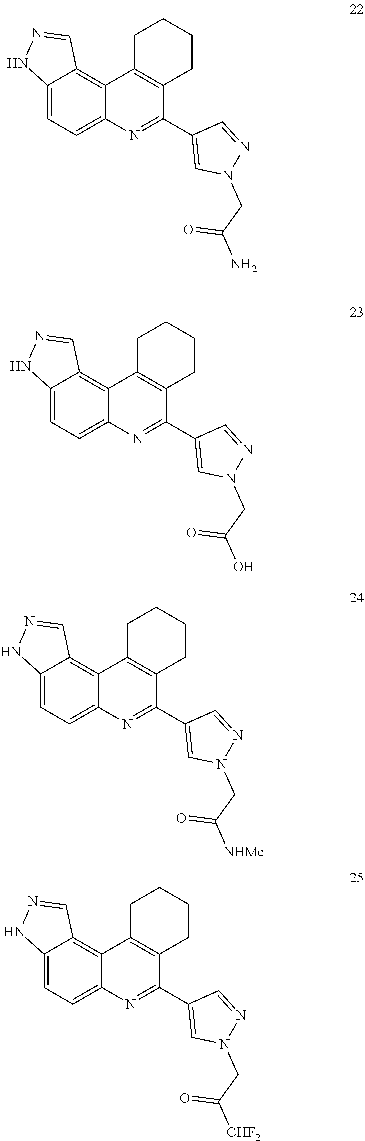 [SMILES: NC(=O)Cn1cc(-c2nc3ccc4[nH]ncc4c3c3c2CCCC3)cn1 ; O=C(O)Cn1cc(-c2nc3ccc4[nH]ncc4c3c3c2CCCC3)cn1 ; CNC(=O)Cn1cc(-c2nc3ccc4[nH]ncc4c3c3c2CCCC3)cn1 ; O=C(Cn1cc(-c2nc3ccc4[nH]ncc4c3c3c2CCCC3)cn1)C(F)F]

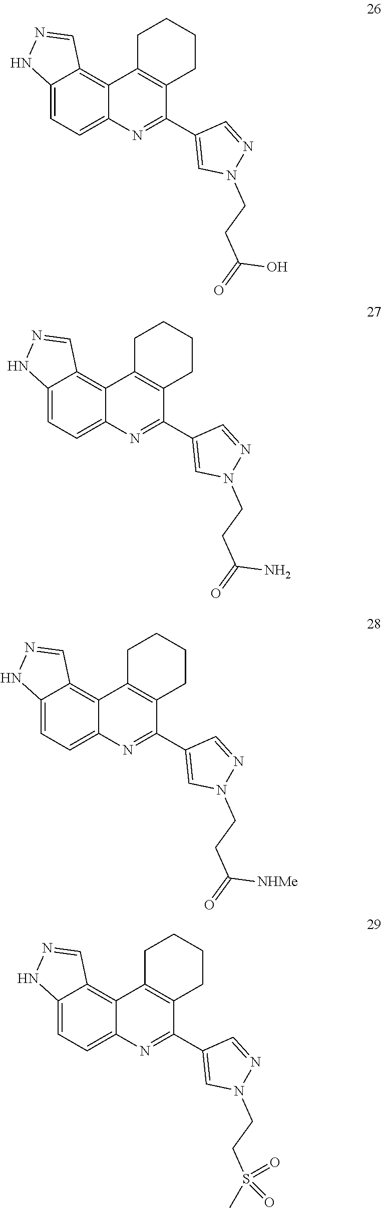

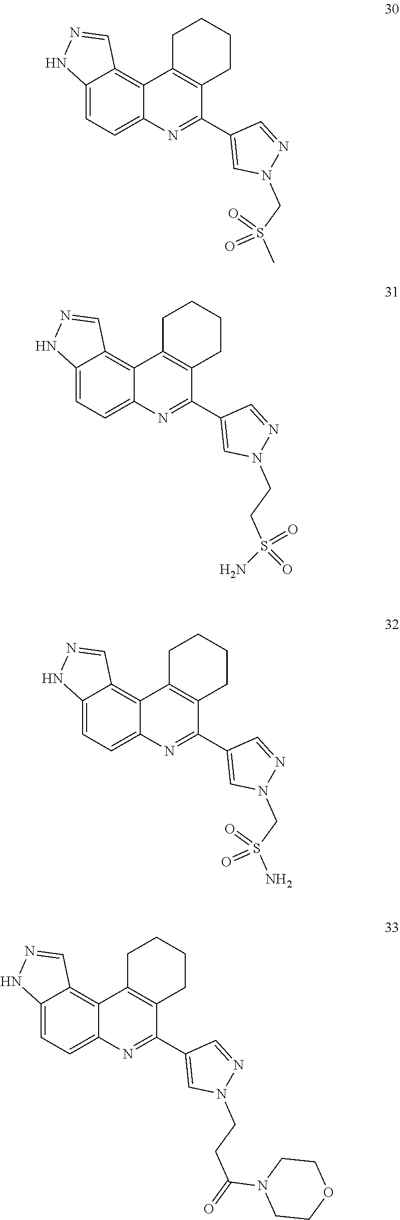 [SMILES: CS(=O)(=O)Cn1cc(-c2nc3ccc4[nH]ncc4c3c3c2CCCC3)cn1 ; NS(=O)(=O)CCn1cc(-c2nc3ccc4[nH]ncc4c3c3c2CCCC3)cn1 ; NS(=O)(=O)Cn1cc(-c2nc3ccc4[nH]ncc4c3c3c2CCCC3)cn1 ; O=C(CCn1cc(-c2nc3ccc4[nH]ncc4c3c3c2CCCC3)cn1)N1CCOCC1]

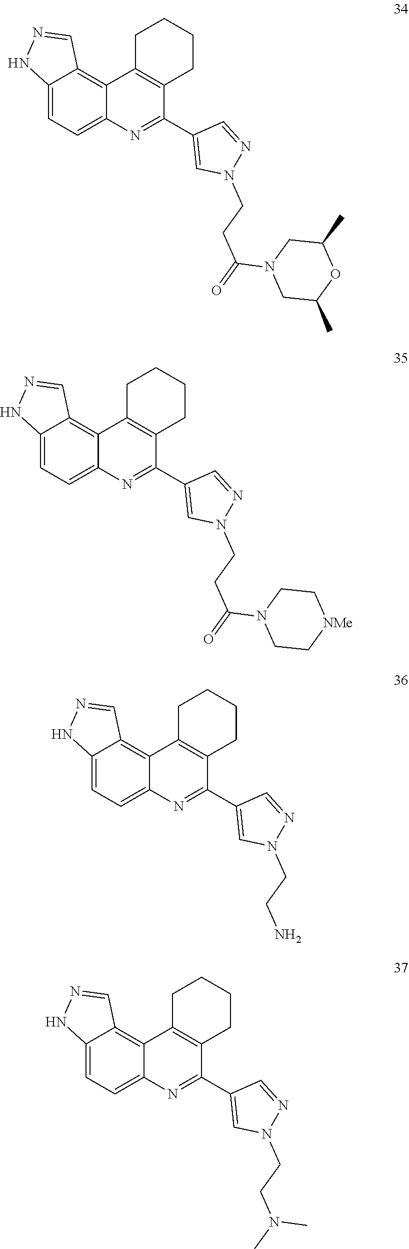 [SMILES: C[C@@H]1CN(C(=O)CCn2cc(-c3nc4ccc5[nH]ncc5c4c4c3CCCC4)cn2)C[C@H](C)O1 ; CN1CCN(C(=O)CCn2cc(-c3nc4ccc5[nH]ncc5c4c4c3CCCC4)cn2)CC1 ; NCCn1cc(-c2nc3ccc4[nH]ncc4c3c3c2CCCC3)cn1 ; CN(C)CCn1cc(-c2nc3ccc4[nH]ncc4c3c3c2CCCC3)cn1]

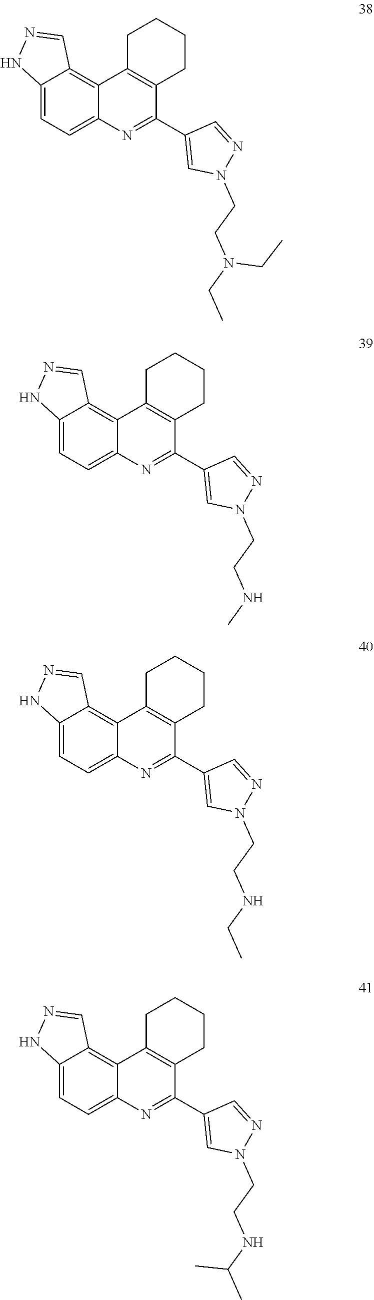 [SMILES: CCN(CC)CCn1cc(-c2nc3ccc4[nH]ncc4c3c3c2CCCC3)cn1 ; CNCCn1cc(-c2nc3ccc4[nH]ncc4c3c3c2CCCC3)cn1 ; CCNCCn1cc(-c2nc3ccc4[nH]ncc4c3c3c2CCCC3)cn1 ; CC(C)NCCn1cc(-c2nc3ccc4[nH]ncc4c3c3c2CCCC3)cn1]

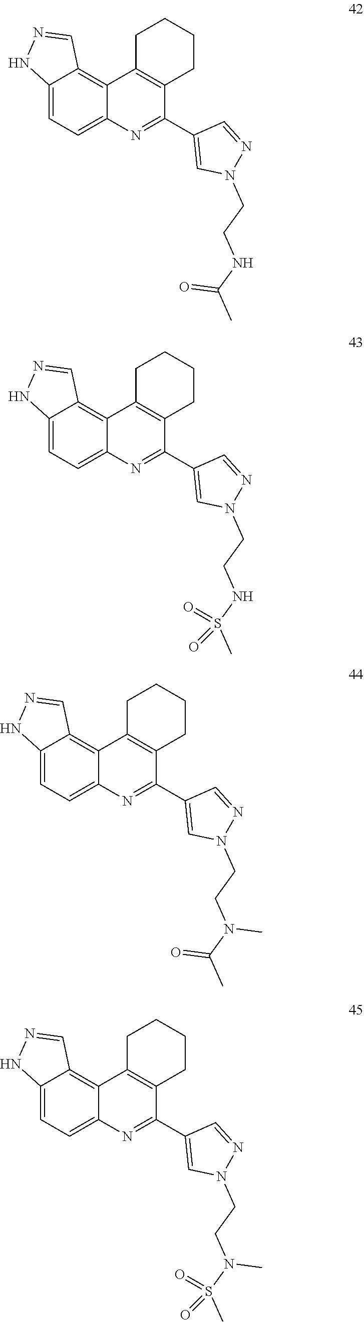 [SMILES: CC(=O)NCCn1cc(-c2nc3ccc4[nH]ncc4c3c3c2CCCC3)cn1 ; CS(=O)(=O)NCCn1cc(-c2nc3ccc4[nH]ncc4c3c3c2CCCC3)cn1 ; CC(=O)N(C)CCn1cc(-c2nc3ccc4[nH]ncc4c3c3c2CCCC3)cn1 ; CN(CCn1cc(-c2nc3ccc4[nH]ncc4c3c3c2CCCC3)cn1)S(C)(=O)=O]

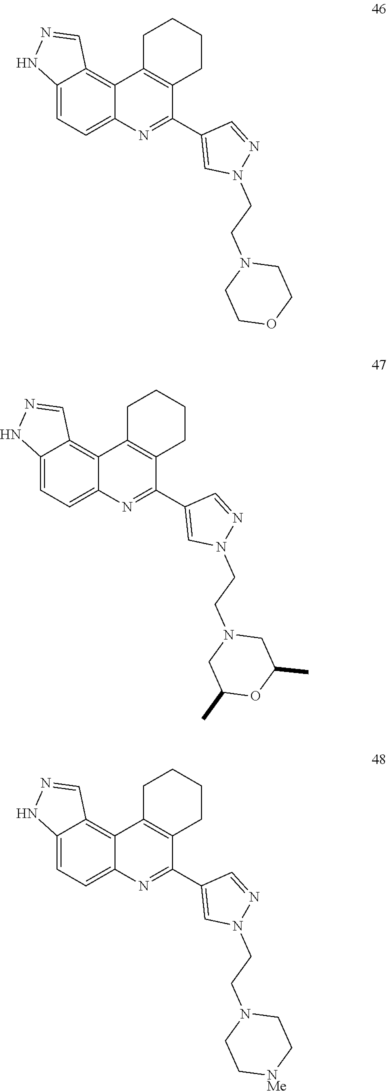 [SMILES: c1nn(CCN2CCOCC2)cc1-c1nc2ccc3[nH]ncc3c2c2c1CCCC2 ; C[C@@H]1CN(CCn2cc(-c3nc4ccc5[nH]ncc5c4c4c3CCCC4)cn2)C[C@H](C)O1 ; CN1CCN(CCn2cc(-c3nc4ccc5[nH]ncc5c4c4c3CCCC4)cn2)CC1]

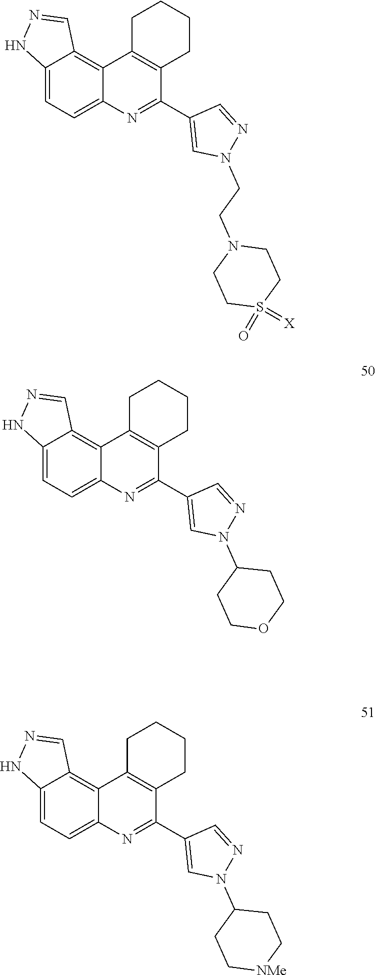

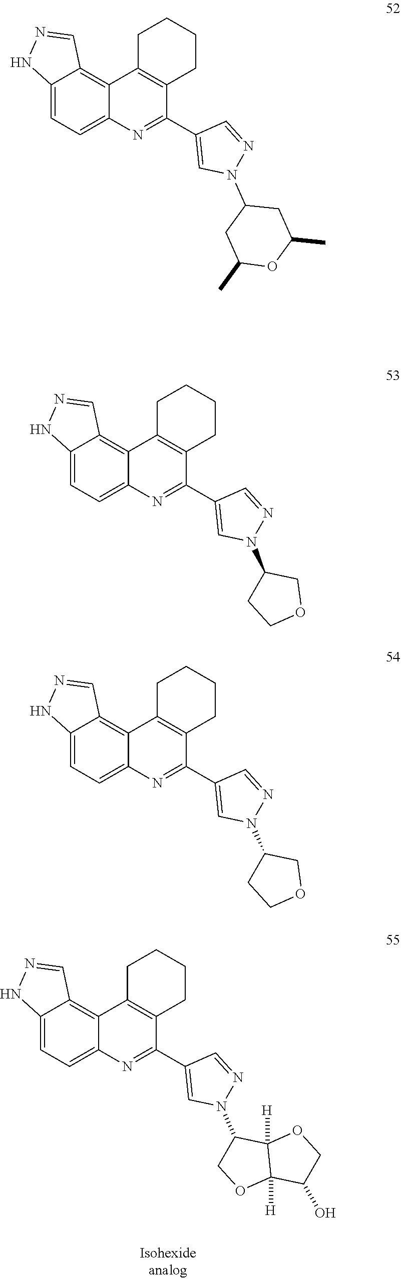

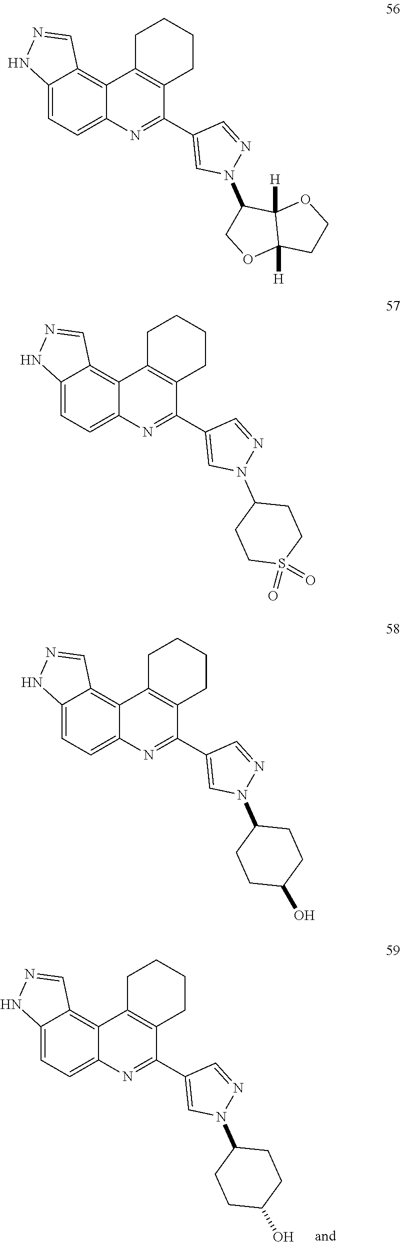

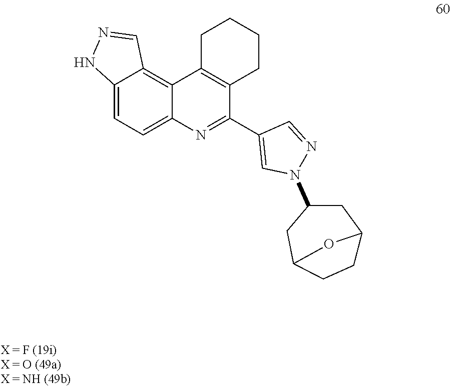

or a pharmaceutically acceptable salt, hydrate, tautomer, or optical isomer thereof.

In some embodiments, the compound of formula (IA) is

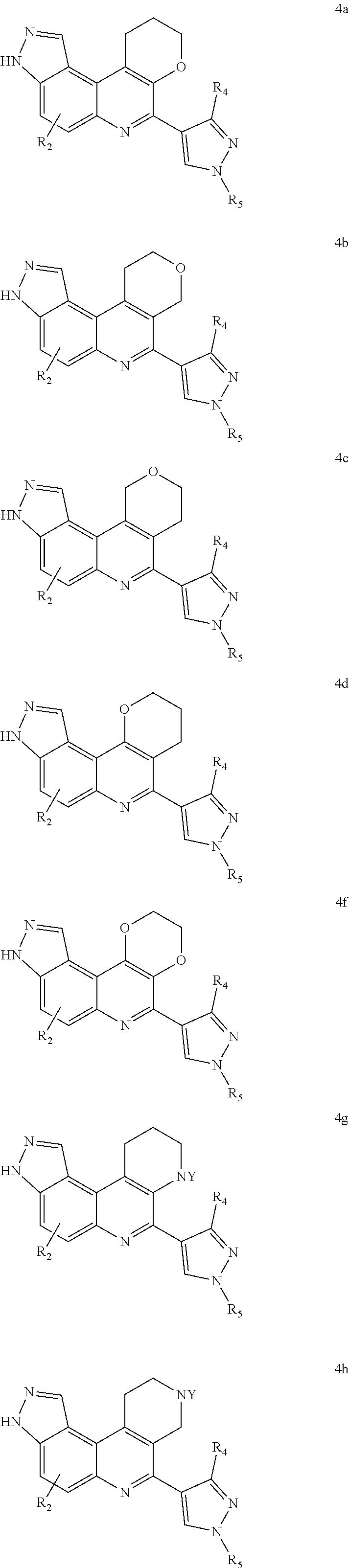

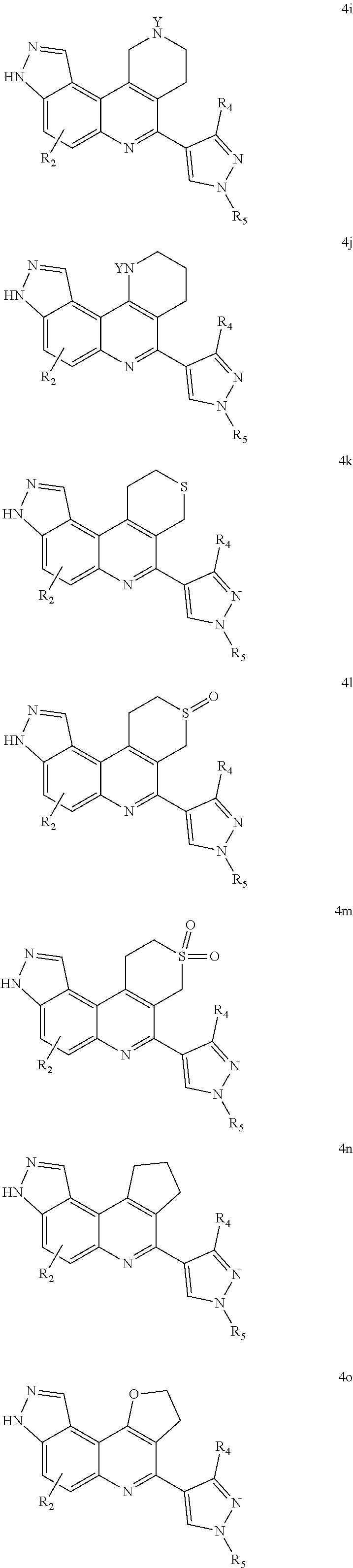

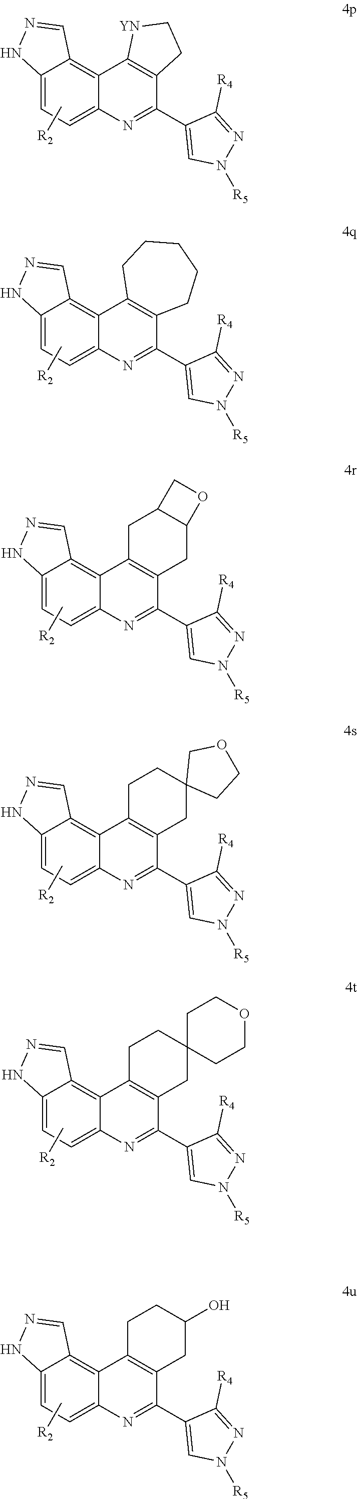

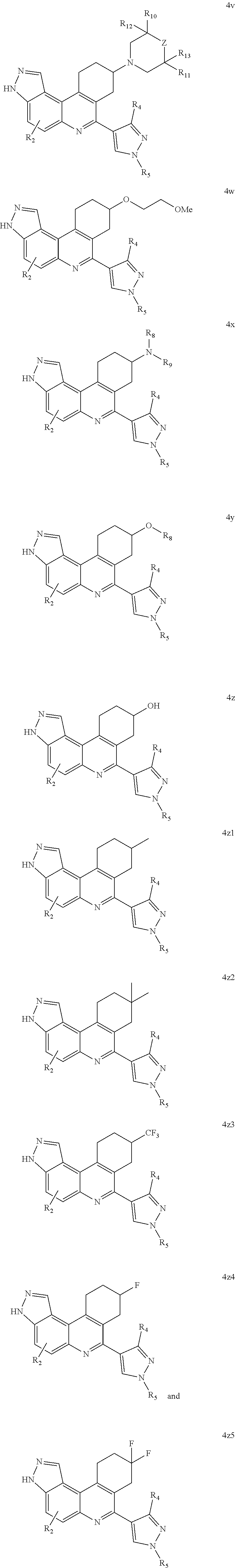

wherein
R₂ is selected from H, alkyl, heteroalkyl, aryl, heteroaryl, alkenyl, alkynyl, and halogen;
each R₄ and R₅ is independently selected from H, alkyl, cycloalkyl, heteroalkyl, heterocycloalkyl, aryl, heteroaryl, alkenyl, alkynyl, and amide, wherein the amide is selected from sulfonamide, benzamide, nicotinamide, picolinamide and a derivative of any of the forgoing, wherein alkyl is optionally substituted with OH, OR₂, halogen, CF₃, CN, NRR′, CONRR′, SO₂R₂, SO₂NRR′, NCOR₂ or NSO₂R₂, wherein each R and R′ is independently selected from H, halogen, CF₃, CN, alkyl, aryl, heteroalkyl, heteroaryl, CONR₂R₂, COR₂, SO₂R₂, (C═O)NR₂, (C═NH)R₂, (C═O)OR₂, C₆H₅R₂, morpholine and piperazine, wherein R₂ is as defined above; or
R and R′, together with the atom to which they are attached, form a 4- to 6-membered cyclic or heterocyclic ring;
each R₈ and R₉ is independently selected from H, methyl, alkyl, and heteroalkyl;
each R₁₀, R₁₁, R₁₂, and R₁₃ is independently selected from H, methyl, alkyl, and heteroalkyl; or R₁₀ and R₁₂, together with the atom to which they are attached, form a 4- to 6-membered cyclic or heterocyclic ring; or
R₁₁ and R₁₃, together with the atom to which they are attached, form a 4- to 6-membered cyclic or heterocyclic ring;
Z is selected from O, S, SO, SO₂, CRR′, CNRR′, COR′ and NR′, wherein each R and R′ is as defined above;
Y is selected from COR₂, SO₂R₂, (C═O)NR₂, (C═NH)R₂, (C═O)OR₂ and C₆H₅R₂, wherein R₂ is as defined above;
or a pharmaceutically acceptable salt, hydrate, tautomer, or optical isomer thereof.

In some embodiments, the formula (IA) comprises:
R₂ is H;
R₄ is H or alkyl;
each X is independently selected from O, S, SO, SO₂, CRR′, CNRR′, COR′, NR′ and Xₙ, wherein n is 0-2; wherein each R and R′ is independently selected from H, halogen, CF₃, CN, alkyl, aryl, heteroalkyl, heteroaryl, CONR₂R₂, COR₂, SO₂R₂, (C═O)NR₂, (C═NH)R₂, (C═O)OR₂, C₆H₅R₂, morpholine and piperazine, wherein R₂ is as defined above; or
R and R′, together with the atom to which they are attached, form a 4- to 6-membered cyclic or heterocyclic ring; and
R₅ is selected from H, alkyl, cycloalkyl, heteroalkyl, heterocycloalkyl, aryl, heteroaryl, alkenyl, alkynyl, and amide, wherein the amide is selected from sulfonamide, benzamide, nicotinamide, picolinamide and a derivative of any of the forgoing; and R₅ is optionally substituted with OH, OR₂, halogen, CF₃, CN, NRR′, CONRR′, SO₂R₂, SO₂NRR′, NCOR₂ or NSO₂R₂, wherein R₂, R and R′ are as defined above; and with the proviso that R₅ is not H, methyl, CF₃, CH₂—CH₂—OH, CH₂—CH₂—C₁, COOH, or optionally substituted phenyl, wherein the substitution on phenyl is selected from OMe, COOMe, and amino;
or a pharmaceutically acceptable salt, hydrate, tautomer, or optical isomer thereof.

In some embodiments, the compound of formula (IA) is

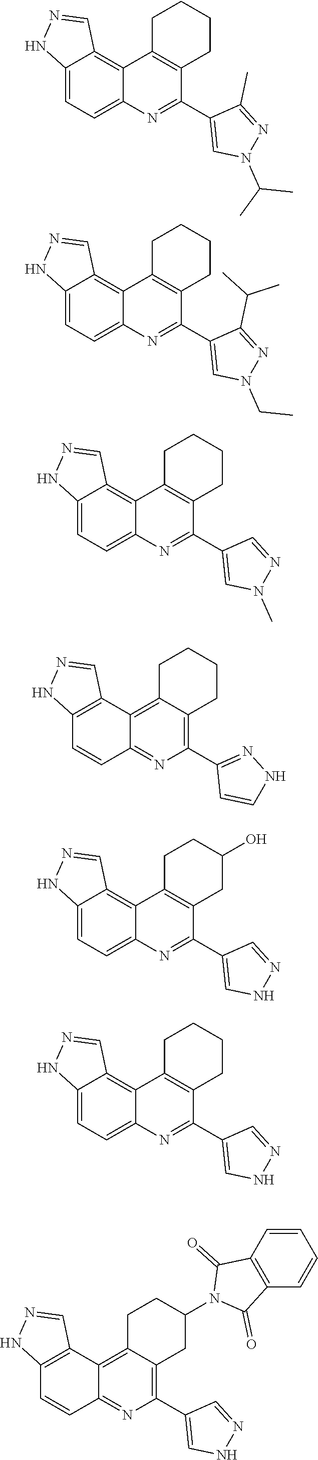

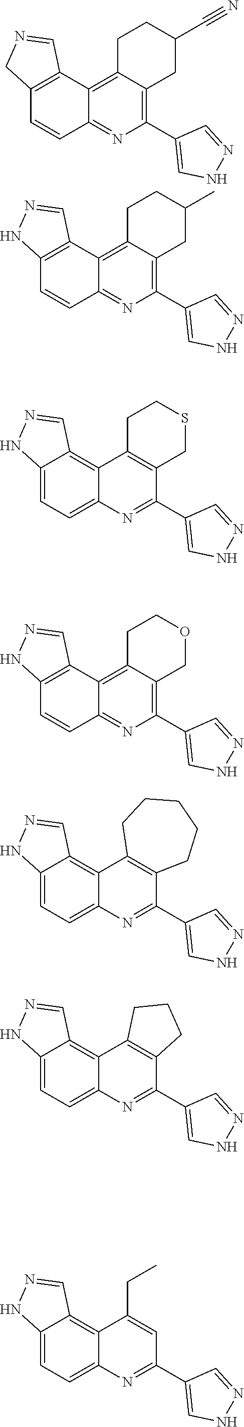

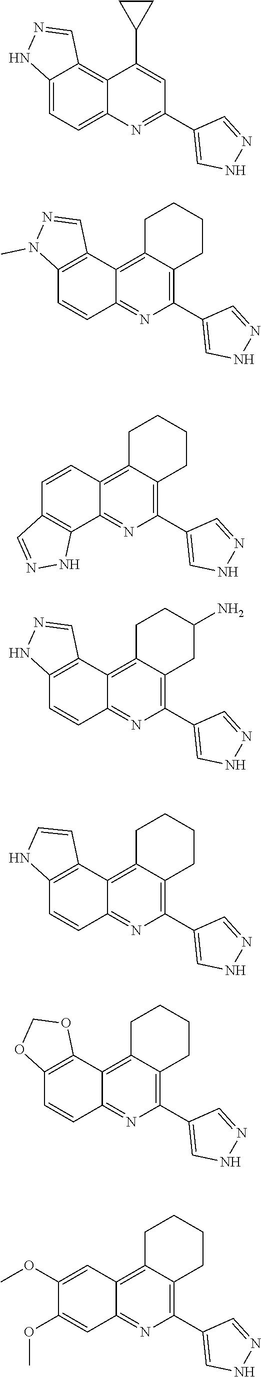

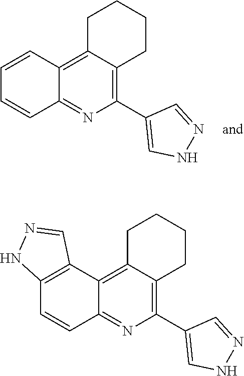 [SMILES: c1ccc2c3c(c(-c4cn[nH]c4)nc2c1)CCCC3 ; "and" ; c1n[nH]cc1-c1nc2ccc3[nH]ncc3c2c2c1CCCC2]

or a pharmaceutically acceptable salt, hydrate, tautomer, or optical isomer thereof.

In some embodiments, the compound of formula (I) is represented by a compound of formula (IB)

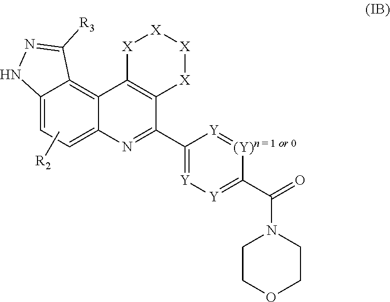

wherein
R₂ is selected from H, alkyl, heteroalkyl, aryl, heteroaryl, alkenyl, alkynyl, and halogen;
R₃ is selected from H, alkyl, heteroalkyl, aryl, heteroaryl, alkenyl, and alkynyl;
each X is independently selected from O, S, SO, SO₂, CRR′, CNRR′, COR′, NR′ and Xₙ, wherein n is 0-2; wherein each R and R′ is independently selected from H, halogen, CF₃, CN, alkyl, aryl, heteroalkyl, heteroaryl, CONR₂R₂, COR₂, SO₂R₂, (C═O)NR₂, (C═NH)R₂, (C═O)OR₂, C₆H₅R₂, morpholine and piperazine, wherein R₂ is as defined above; or
R and R′, together with the atom to which they are attached, form a 4- to 6-membered cyclic or heterocyclic ring;
Y is N or CR₂, wherein R₂ is as defined above;
n is 0 or 1;
Q is selected from NR″, CR₂R₂, N(COR₂), N(SO₂)R₂, N(C═O)NR₂, N(C═NH)R₂, and N(C═O)OR₂, with the proviso that when Q is NR″, R″ is not H, methyl, isopropyl, CH₂CH₂OH, COCH₃ or SO₂-methyl; and when Q is CR₂R₂, R₂ is not H or dimethyl amine;
or a pharmaceutically acceptable salt, hydrate, tautomer, or optical isomer thereof.

In some embodiments, the compound of formula (IB) is

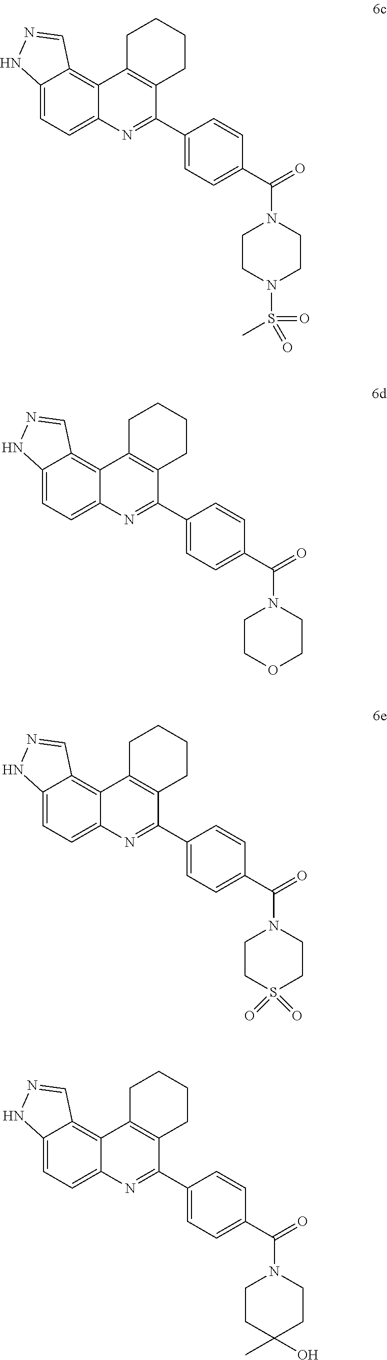 [SMILES: CS(=O)(=O)N1CCN(C(=O)c2ccc(-c3nc4ccc5[nH]ncc5c4c4c3CCCC4)cc2)CC1 ; O=C(c1ccc(-c2nc3ccc4[nH]ncc4c3c3c2CCCC3)cc1)N1CCOCC1 ; O=C(c1ccc(-c2nc3ccc4[nH]ncc4c3c3c2CCCC3)cc1)N1CCS(=O)(=O)CC1 ; CC1(O)CCN(C(=O)c2ccc(-c3nc4ccc5[nH]ncc5c4c4c3CCCC4)cc2)CC1]

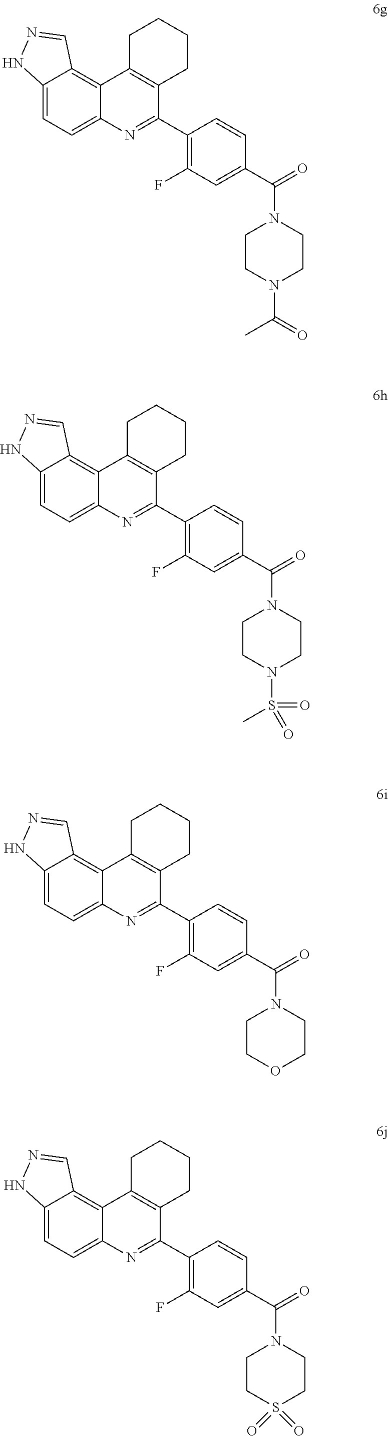 [SMILES: CC(=O)N1CCN(C(=O)c2ccc(-c3nc4ccc5[nH]ncc5c4c4c3CCCC4)c(F)c2)CC1 ; CS(=O)(=O)N1CCN(C(=O)c2ccc(-c3nc4ccc5[nH]ncc5c4c4c3CCCC4)c(F)c2)CC1 ; O=C(c1ccc(-c2nc3ccc4[nH]ncc4c3c3c2CCCC3)c(F)c1)N1CCOCC1 ; O=C(c1ccc(-c2nc3ccc4[nH]ncc4c3c3c2CCCC3)c(F)c1)N1CCS(=O)(=O)CC1]

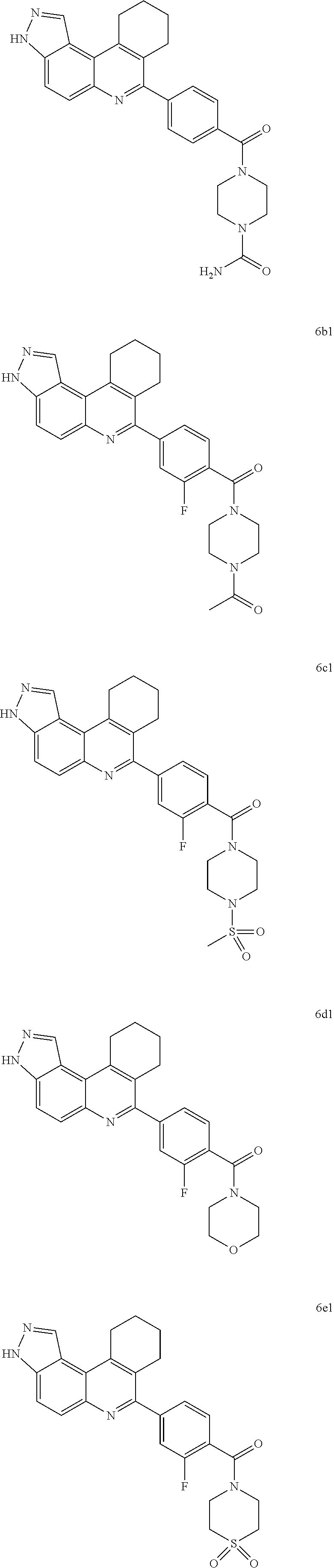 [SMILES: NC(=O)N1CCN(C(=O)c2ccc(-c3nc4ccc5[nH]ncc5c4c4c3CCCC4)cc2)CC1 ; CC(=O)N1CCN(C(=O)c2ccc(-c3nc4ccc5[nH]ncc5c4c4c3CCCC4)cc2F)CC1 ; CS(=O)(=O)N1CCN(C(=O)c2ccc(-c3nc4ccc5[nH]ncc5c4c4c3CCCC4)cc2F)CC1 ; O=C(c1ccc(-c2nc3ccc4[nH]ncc4c3c3c2CCCC3)cc1F)N1CCOCC1 ; O=C(c1ccc(-c2nc3ccc4[nH]ncc4c3c3c2CCCC3)cc1F)N1CCS(=O)(=O)CC1]

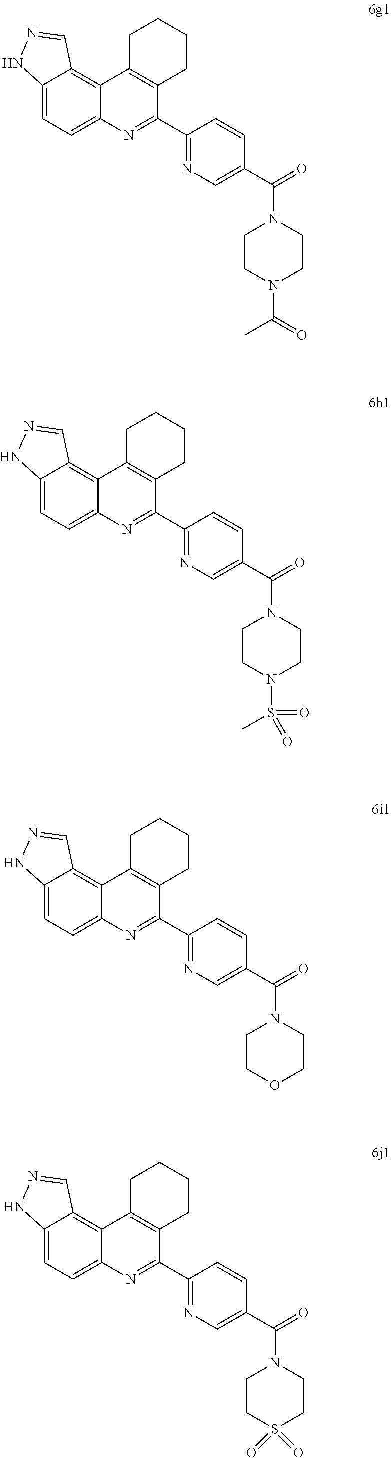 [SMILES: CC(=O)N1CCN(C(=O)c2ccc(-c3nc4ccc5[nH]ncc5c4c4c3CCCC4)nc2)CC1 ; CS(=O)(=O)N1CCN(C(=O)c2ccc(-c3nc4ccc5[nH]ncc5c4c4c3CCCC4)nc2)CC1 ; O=C(c1ccc(-c2nc3ccc4[nH]ncc4c3c3c2CCCC3)nc1)N1CCOCC1 ; O=C(c1ccc(-c2nc3ccc4[nH]ncc4c3c3c2CCCC3)nc1)N1CCS(=O)(=O)CC1]

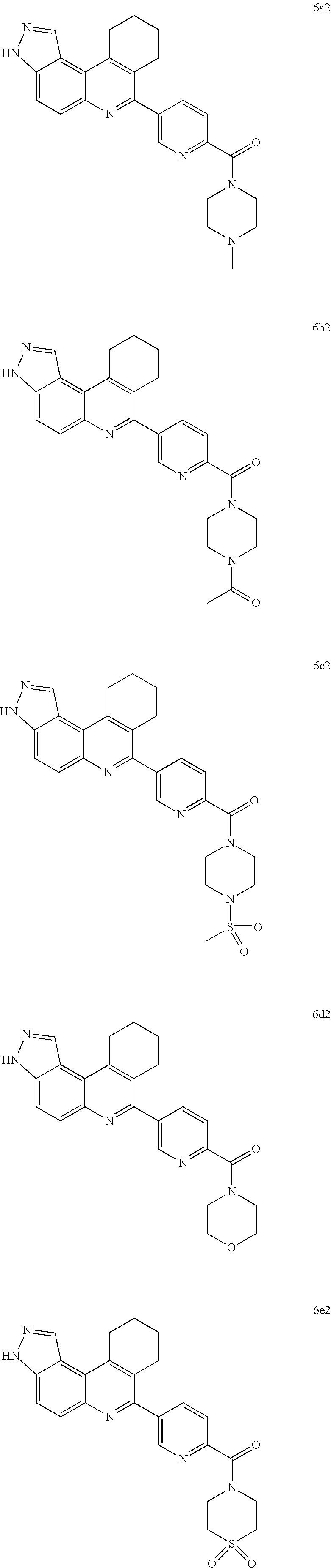 [SMILES: CN1CCN(C(=O)c2ccc(-c3nc4ccc5[nH]ncc5c4c4c3CCCC4)cn2)CC1 ; CC(=O)N1CCN(C(=O)c2ccc(-c3nc4ccc5[nH]ncc5c4c4c3CCCC4)cn2)CC1 ; CS(=O)(=O)N1CCN(C(=O)c2ccc(-c3nc4ccc5[nH]ncc5c4c4c3CCCC4)cn2)CC1 ; O=C(c1ccc(-c2nc3ccc4[nH]ncc4c3c3c2CCCC3)cn1)N1CCOCC1 ; O=C(c1ccc(-c2nc3ccc4[nH]ncc4c3c3c2CCCC3)cn1)N1CCS(=O)(=O)CC1]

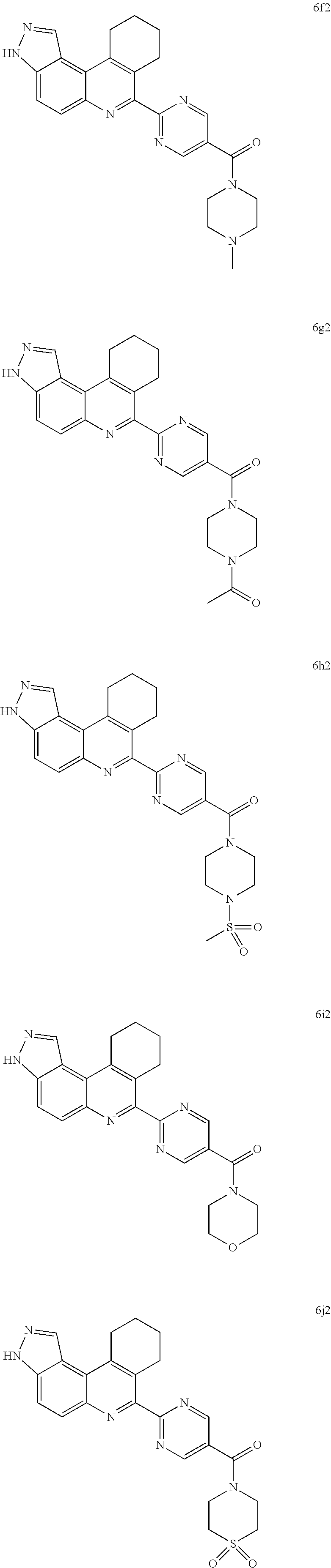 [SMILES: CN1CCN(C(=O)c2cnc(-c3nc4ccc5[nH]ncc5c4c4c3CCCC4)nc2)CC1 ; CC(=O)N1CCN(C(=O)c2cnc(-c3nc4ccc5[nH]ncc5c4c4c3CCCC4)nc2)CC1 ; CS(=O)(=O)N1CCN(C(=O)c2cnc(-c3nc4ccc5[nH]ncc5c4c4c3CCCC4)nc2)CC1 ; O=C(c1cnc(-c2nc3ccc4[nH]ncc4c3c3c2CCCC3)nc1)N1CCOCC1 ; O=C(c1cnc(-c2nc3ccc4[nH]ncc4c3c3c2CCCC3)nc1)N1CCS(=O)(=O)CC1]

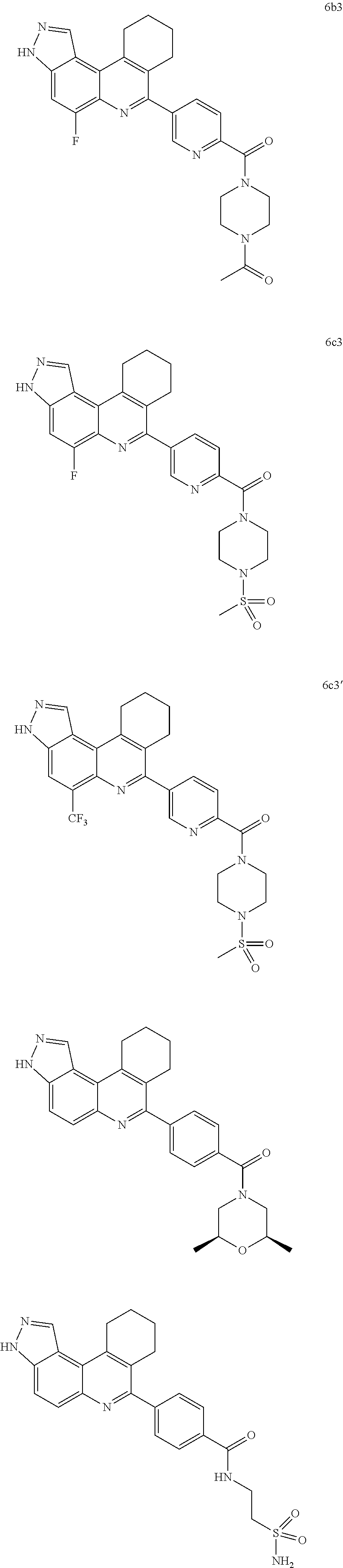 [SMILES: CC(=O)N1CCN(C(=O)c2ccc(-c3nc4c(F)cc5[nH]ncc5c4c4c3CCCC4)cn2)CC1 ; CS(=O)(=O)N1CCN(C(=O)c2ccc(-c3nc4c(F)cc5[nH]ncc5c4c4c3CCCC4)cn2)CC1 ; CS(=O)(=O)N1CCN(C(=O)c2ccc(-c3nc4c(C(F)(F)F)cc5[nH]ncc5c4c4c3CCCC4)cn2)CC1 ; C[C@@H]1CN(C(=O)c2ccc(-c3nc4ccc5[nH]ncc5c4c4c3CCCC4)cc2)C[C@H](C)O1 ; NS(=O)(=O)CCNC(=O)c1ccc(-c2nc3ccc4[nH]ncc4c3c3c2CCCC3)cc1]

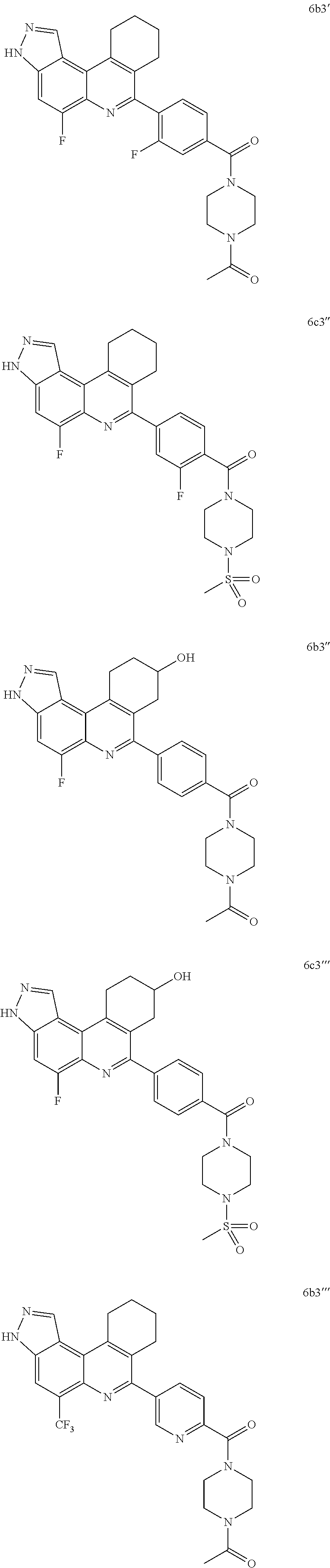 [SMILES: CC(=O)N1CCN(C(=O)c2ccc(-c3nc4c(F)cc5[nH]ncc5c4c4c3CCCC4)c(F)c2)CC1 ; CS(=O)(=O)N1CCN(C(=O)c2ccc(-c3nc4c(F)cc5[nH]ncc5c4c4c3CCCC4)cc2F)CC1 ; CC(=O)N1CCN(C(=O)c2ccc(-c3nc4c(F)cc5[nH]ncc5c4c4c3CC(O)CC4)cc2)CC1 ; CS(=O)(=O)N1CCN(C(=O)c2ccc(-c3nc4c(F)cc5[nH]ncc5c4c4c3CC(O)CC4)cc2)CC1 ; CC(=O)N1CCN(C(=O)c2ccc(-c3nc4c(C(F)(F)F)cc5[nH]ncc5c4c4c3CCCC4)cn2)CC1]

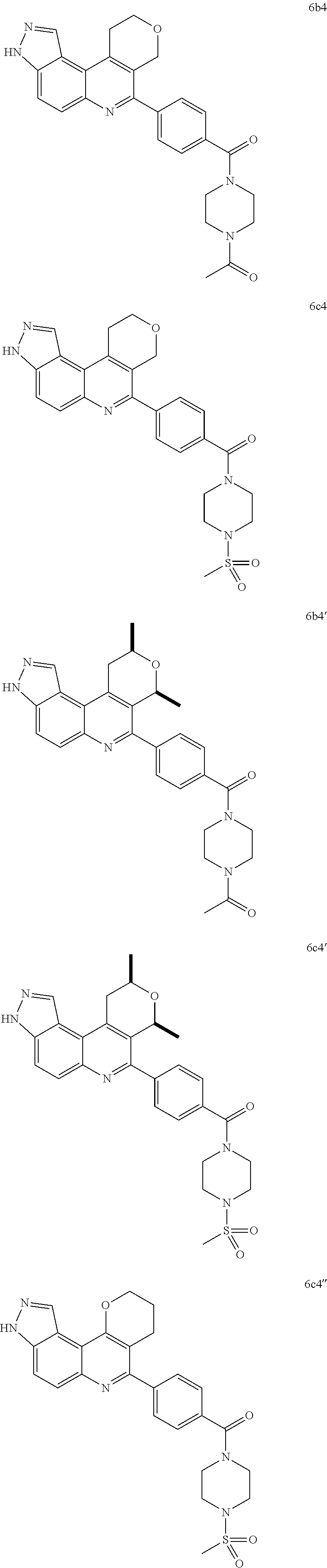 [SMILES: CC(=O)N1CCN(C(=O)c2ccc(-c3nc4ccc5[nH]ncc5c4c4c3COCC4)cc2)CC1 ; CS(=O)(=O)N1CCN(C(=O)c2ccc(-c3nc4ccc5[nH]ncc5c4c4c3COCC4)cc2)CC1 ; CC(=O)N1CCN(C(=O)c2ccc(-c3nc4ccc5[nH]ncc5c4c4c3[C@H](C)O[C@H](C)C4)cc2)CC1 ; C[C@@H]1Cc2c(c(-c3ccc(C(=O)N4CCN(S(C)(=O)=O)CC4)cc3)nc3ccc4[nH]ncc4c23)[C@H](C)O1 ; CS(=O)(=O)N1CCN(C(=O)c2ccc(-c3nc4ccc5[nH]ncc5c4c4c3CCCO4)cc2)CC1]

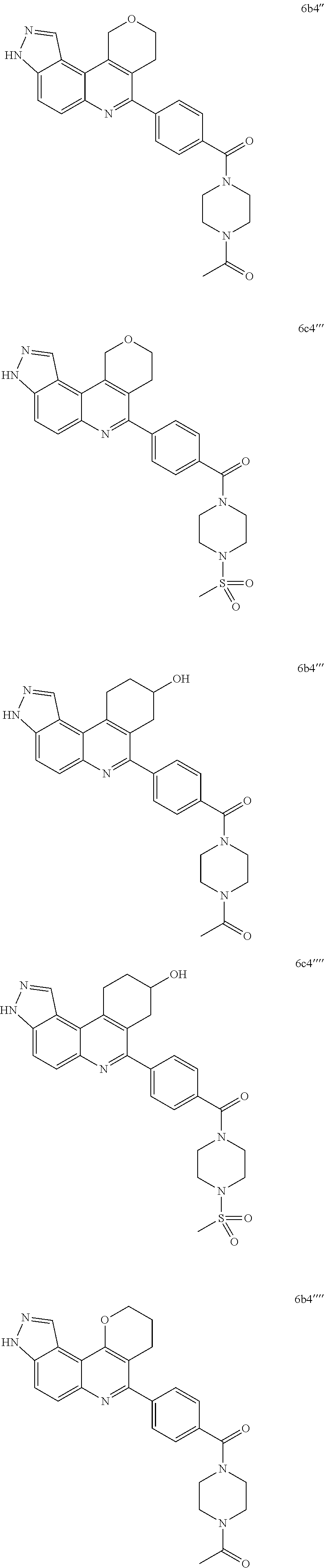 [SMILES: CC(=O)N1CCN(C(=O)c2ccc(-c3nc4ccc5[nH]ncc5c4c4c3CCOC4)cc2)CC1 ; CS(=O)(=O)N1CCN(C(=O)c2ccc(-c3nc4ccc5[nH]ncc5c4c4c3CCOC4)cc2)CC1 ; CC(=O)N1CCN(C(=O)c2ccc(-c3nc4ccc5[nH]ncc5c4c4c3CC(O)CC4)cc2)CC1 ; CS(=O)(=O)N1CCN(C(=O)c2ccc(-c3nc4ccc5[nH]ncc5c4c4c3CC(O)CC4)cc2)CC1 ; CC(=O)N1CCN(C(=O)c2ccc(-c3nc4ccc5[nH]ncc5c4c4c3CCCO4)cc2)CC1]

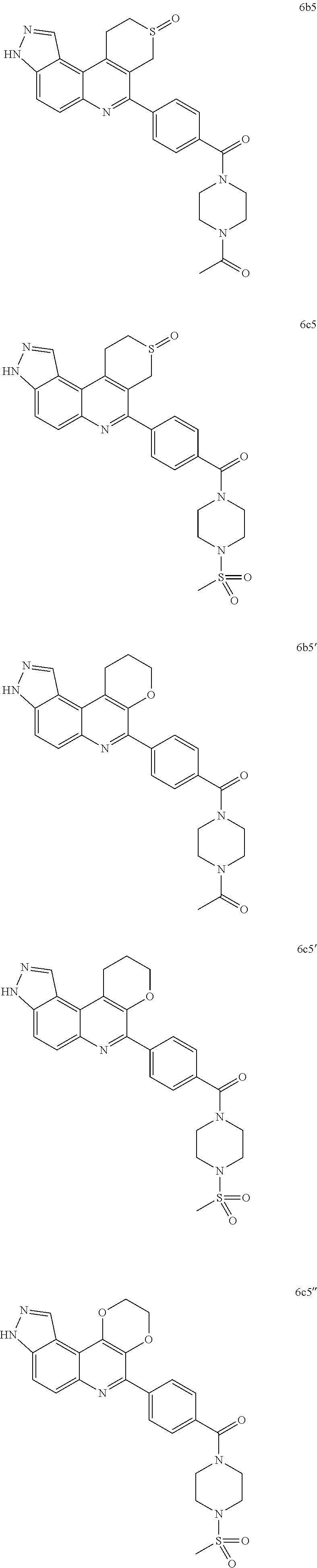 [SMILES: CC(=O)N1CCN(C(=O)c2ccc(-c3nc4ccc5[nH]ncc5c4c4c3CS(=O)CC4)cc2)CC1 ; CS(=O)(=O)N1CCN(C(=O)c2ccc(-c3nc4ccc5[nH]ncc5c4c4c3CS(=O)CC4)cc2)CC1 ; CC(=O)N1CCN(C(=O)c2ccc(-c3nc4ccc5[nH]ncc5c4c4c3OCCC4)cc2)CC1 ; CS(=O)(=O)N1CCN(C(=O)c2ccc(-c3nc4ccc5[nH]ncc5c4c4c3OCCC4)cc2)CC1 ; CS(=O)(=O)N1CCN(C(=O)c2ccc(-c3nc4ccc5[nH]ncc5c4c4c3OCCO4)cc2)CC1]

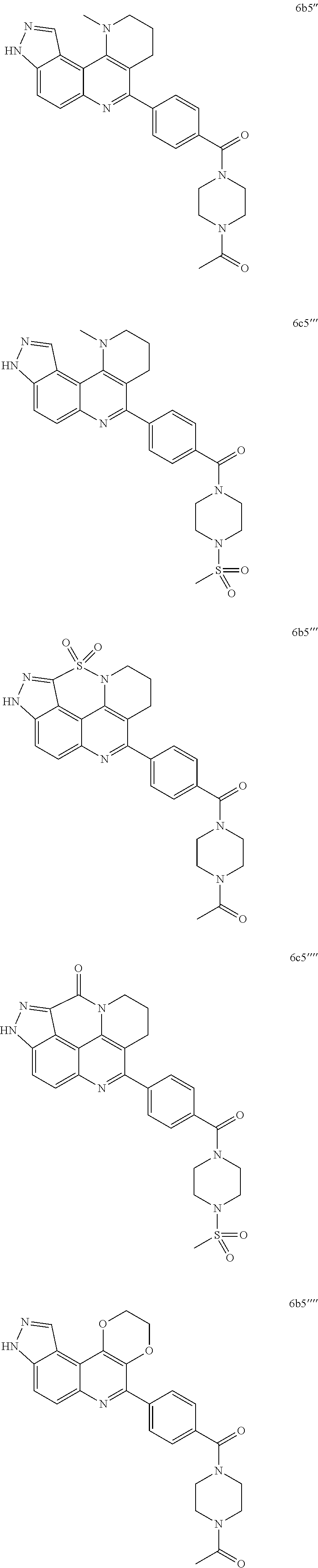 [SMILES: CC(=O)N1CCN(C(=O)c2ccc(-c3nc4ccc5[nH]ncc5c4c4c3CCCN4C)cc2)CC1 ; CN1CCCc2c(-c3ccc(C(=O)N4CCN(S(C)(=O)=O)CC4)cc3)nc3ccc4[nH]ncc4c3c21 ; CC(=O)N1CCN(C(=O)c2ccc(-c3nc4ccc5[nH]nc6c5c4c4c3CCCN4S6(=O)=O)cc2)CC1 ; CS(=O)(=O)N1CCN(C(=O)c2ccc(-c3nc4ccc5[nH]nc6c(=O)n7c(c3CCC7)c4c56)cc2)CC1 ; CC(=O)N1CCN(C(=O)c2ccc(-c3nc4ccc5[nH]ncc5c4c4c3OCCO4)cc2)CC1]

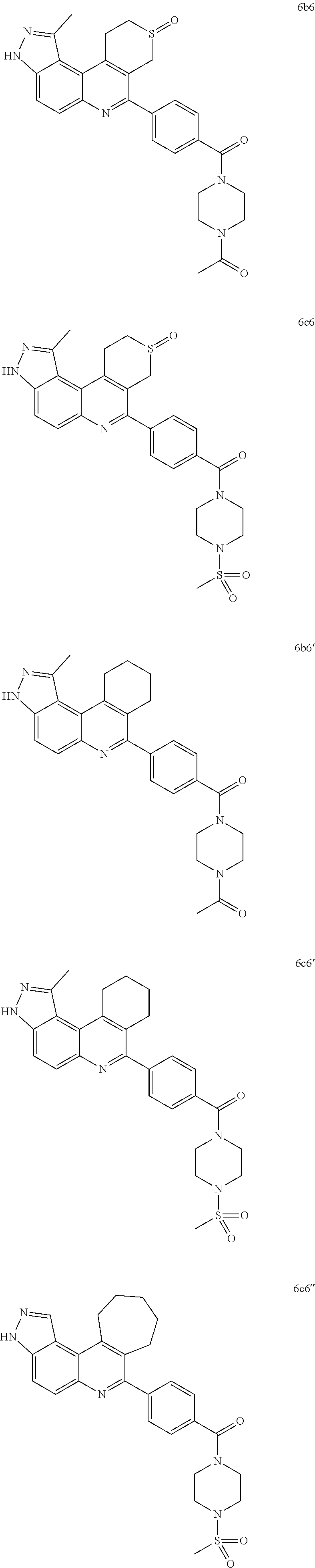 [SMILES: CC(=O)N1CCN(C(=O)c2ccc(-c3nc4ccc5[nH]nc(C)c5c4c4c3CS(=O)CC4)cc2)CC1 ; Cc1n[nH]c2ccc3nc(-c4ccc(C(=O)N5CCN(S(C)(=O)=O)CC5)cc4)c4c(c3c12)CCS(=O)C4 ; CC(=O)N1CCN(C(=O)c2ccc(-c3nc4ccc5[nH]nc(C)c5c4c4c3CCCC4)cc2)CC1 ; Cc1n[nH]c2ccc3nc(-c4ccc(C(=O)N5CCN(S(C)(=O)=O)CC5)cc4)c4c(c3c12)CCCC4 ; CS(=O)(=O)N1CCN(C(=O)c2ccc(-c3nc4ccc5[nH]ncc5c4c4c3CCCCC4)cc2)CC1]

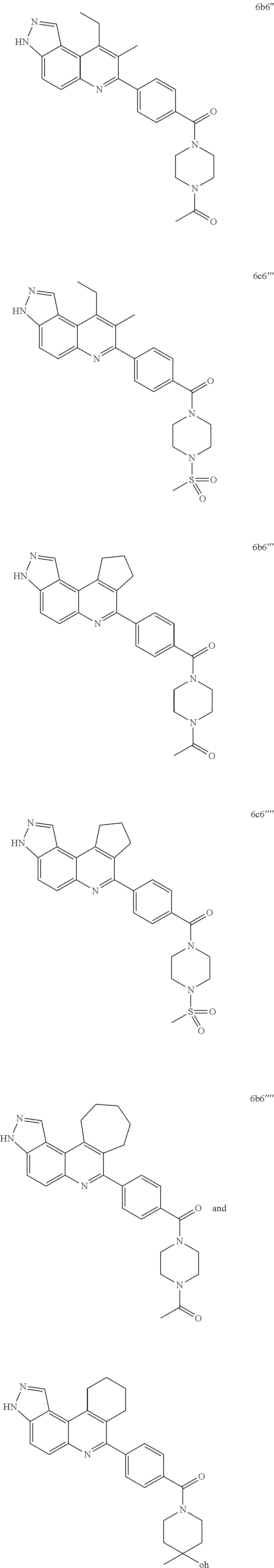

or a pharmaceutically acceptable salt, hydrate, tautomer, or optical isomer thereof.

In some embodiments, compounds of formula (I) is

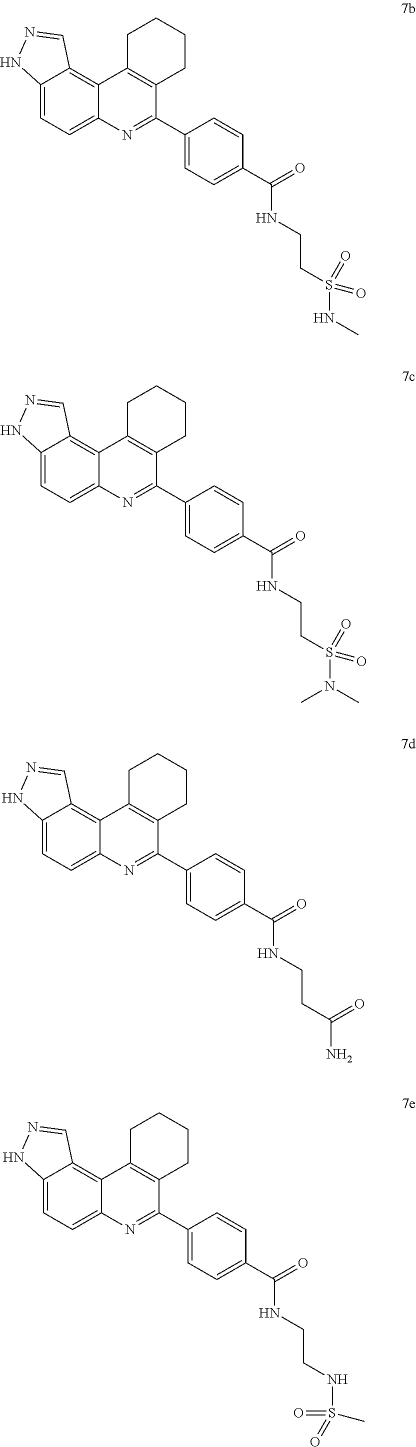 [SMILES: CNS(=O)(=O)CCNC(=O)c1ccc(-c2nc3ccc4[nH]ncc4c3c3c2CCCC3)cc1 ; CN(C)S(=O)(=O)CCNC(=O)c1ccc(-c2nc3ccc4[nH]ncc4c3c3c2CCCC3)cc1 ; NC(=O)CCNC(=O)c1ccc(-c2nc3ccc4[nH]ncc4c3c3c2CCCC3)cc1 ; CS(=O)(=O)NCCNC(=O)c1ccc(-c2nc3ccc4[nH]ncc4c3c3c2CCCC3)cc1]

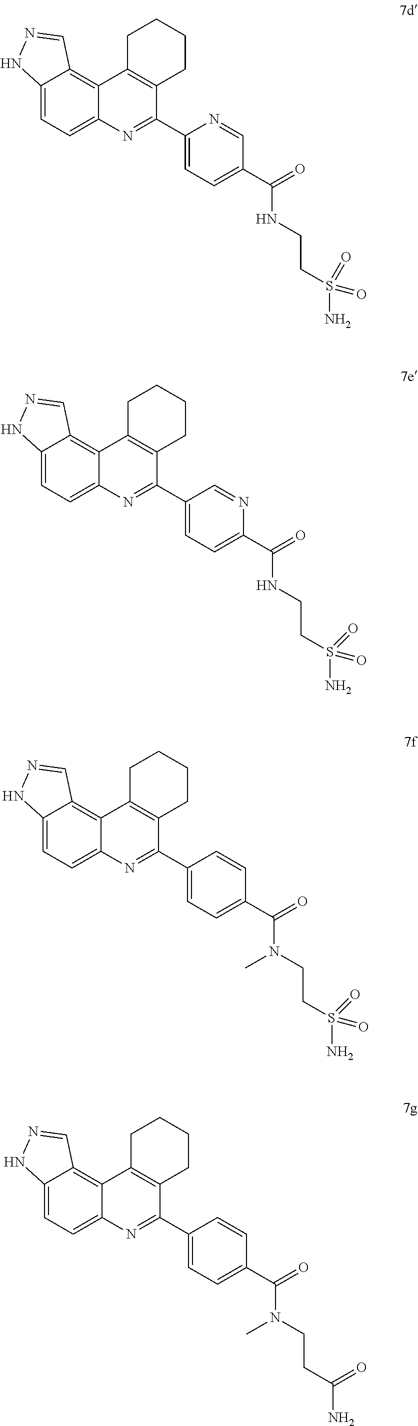 [SMILES: NS(=O)(=O)CCNC(=O)c1ccc(-c2nc3ccc4[nH]ncc4c3c3c2CCCC3)nc1 ; NS(=O)(=O)CCNC(=O)c1ccc(-c2nc3ccc4[nH]ncc4c3c3c2CCCC3)cn1 ; CN(CCS(N)(=O)=O)C(=O)c1ccc(-c2nc3ccc4[nH]ncc4c3c3c2CCCC3)cc1 ; CN(CCC(N)=O)C(=O)c1ccc(-c2nc3ccc4[nH]ncc4c3c3c2CCCC3)cc1]

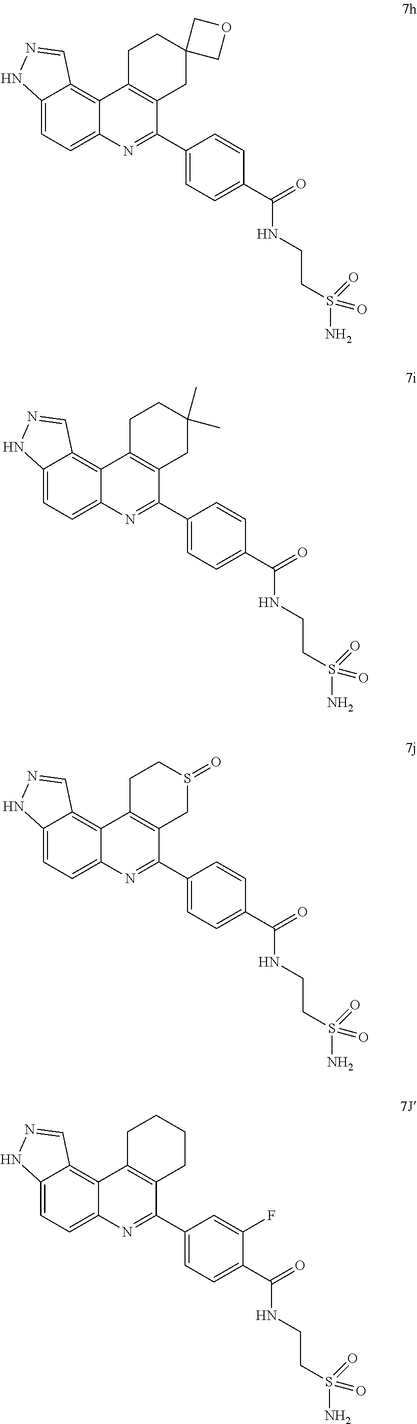 [SMILES: NS(=O)(=O)CCNC(=O)c1ccc(-c2nc3ccc4[nH]ncc4c3c3c2CC2(CC3)COC2)cc1 ; CC1(C)CCc2c(c(-c3ccc(C(=O)NCCS(N)(=O)=O)cc3)nc3ccc4[nH]ncc4c23)C1 ; NS(=O)(=O)CCNC(=O)c1ccc(-c2nc3ccc4[nH]ncc4c3c3c2CS(=O)CC3)cc1 ; NS(=O)(=O)CCNC(=O)c1ccc(-c2nc3ccc4[nH]ncc4c3c3c2CCCC3)cc1F]

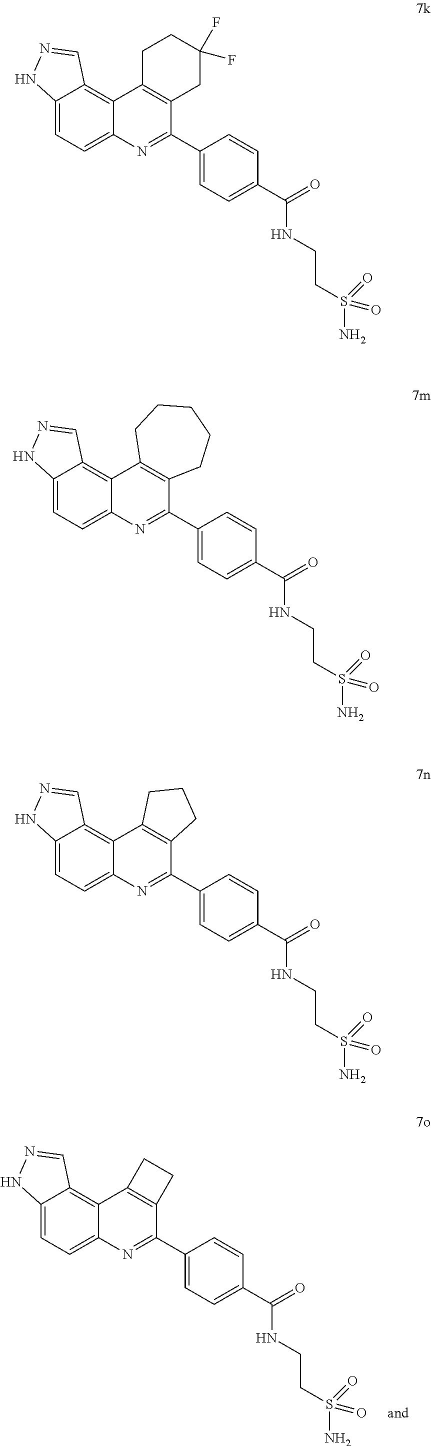 [SMILES: NS(=O)(=O)CCNC(=O)c1ccc(-c2nc3ccc4[nH]ncc4c3c3c2CC(F)(F)CC3)cc1 ; NS(=O)(=O)CCNC(=O)c1ccc(-c2nc3ccc4[nH]ncc4c3c3c2CCCCC3)cc1 ; NS(=O)(=O)CCNC(=O)c1ccc(-c2nc3ccc4[nH]ncc4c3c3c2CCC3)cc1 ; NS(=O)(=O)CCNC(=O)c1ccc(-c2nc3ccc4[nH]ncc4c3c3c2CC3)cc1]

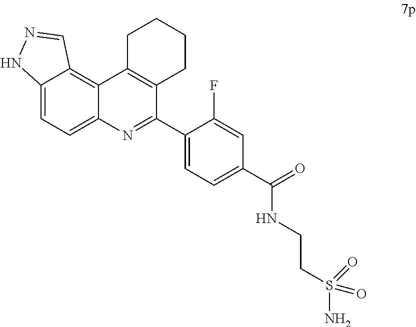 [SMILES: NS(=O)(=O)CCNC(=O)c1ccc(-c2nc3ccc4[nH]ncc4c3c3c2CCCC3)c(F)c1]

or a pharmaceutically acceptable salt, hydrate, tautomer, or optical isomer thereof.

Provided is a method of treating or inhibiting disease associated with overacting of Stimulator of Interferon Genes (STING) comprising administering to a patient a therapeutically effective amount of a compound of formula (IA) or a pharmaceutically acceptable salt, hydrate, tautomer, or optical isomer thereof or a pharmaceutical composition comprising the compound and a pharmaceutically acceptable carrier, excipient, or diluent.

Provided is a method of treating or inhibiting disease associated with overacting of Stimulator of Interferon Genes (STING) comprising administering to a patient a therapeutically effective amount of a compound of formula (IB) or a pharmaceutically acceptable salt, hydrate, tautomer, or optical isomer thereof or a pharmaceutical composition comprising the compound and a pharmaceutically acceptable carrier, excipient, or diluent.

The term “substituted” refers to a functional group in which one or more hydrogen atoms contained therein are replaced by one or more non-hydrogen atoms. The term “functional group” or “substituent” refers to a group that can be or is substituted onto a molecule. Examples of substituents or functional groups include, but are not limited to, a halo (e.g., F, Cl, Br, and I); an oxygen atom in groups such as hydroxyl groups, alkoxy groups, aryloxy groups, aralkyloxy groups, oxo (carbonyl) groups, and carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

Non-limiting examples of substituents, which can be bonded to a substituted carbon atom (or other atom, such as nitrogen) include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, (CH₂)₀₋₂P(O)OR₂, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)C(O)OR, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R wherein R can be hydrogen or a carbon-based moiety, and wherein the carbon-based moiety can itself be further substituted; for example, where R can be hydrogen, alkyl, acyl, cycloalkyl, aryl, aralkyl, heterocyclyl, heteroaryl, or heteroarylalkyl, any alkyl, acyl, cycloalkyl, aryl, aralkyl, heterocyclyl, heteroaryl, or heteroarylalkyl or R can be independently mono- or multi-substituted; or when two R groups bonded to a nitrogen atom or to adjacent nitrogen atoms can, together with the nitrogen atom or atoms to which they are bonded, form a heterocyclyl, the heterocycle can be mono- or independently multi-substituted.

The term “alkyl” refers to substituted and unsubstituted straight-chain and branched alkyl groups and cycloalkyl groups having from 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight-chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. The term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “heteroalkyl,” refers to a stable straight-chain or branched or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P. S. B, As, and Si may be placed at any interior position of the heteroalkyl group or at the position at which the alkyl group is attached to the remainder of the molecule. Examples include, but are not limited to: —CH—CH₂—OCH₃—, —CH₂—CH₂—NH—CH₃, —CH₂—CH₂—N(CH)—CH₃, —CH₂—S—CH₂—CH₃, —CH—CH₂—S(O)—CH₃, —CH₂—CH₂—S(O)—CH₃, —CH═CH OCH₃, —Si(CH₃), CH₂—CH═N—OCH₃, —CH═CH—N(CH)—CH₃, —O—CH₃, —O—CH₂—CH₃, and —CN. Up to two or three heteroatoms may be consecutive, such as, for example, —CH₂—NH—OCH; and —CHO—Si(CH₃)₃. A heteroalkyl moiety may include at least one heteroatom (e.g., O, N, S, Si or P).

The term “alkenyl” refers to substituted and unsubstituted straight-chain and branched divalent alkenyl and cycloalkenyl groups having from 2 to 20 carbon atoms (C₂-C₂₀), 2 to 12 carbons (C₂-C₁₂), 2 to 8 carbon atoms (C₂-C₈) or, in some embodiments, from 2 to 4 carbon atoms (C₂-C₄) and at least one carbon-carbon double bond. Examples of straight-chain alkenyl groups include those with from 2 to 8 carbon atoms, such as —CH═CH—, —CH═CHCH₂—, and the like. Examples of branched alkenyl groups include, but are not limited to, —CH═C(CH₃)— and the like.

The term “alkynyl” refers to an unsaturated monovalent chain of carbon atoms, including at least one triple bond, which may be optionally branched. In various embodiments that include alkynyl, illustrative examples include lower alkynyl, such as C₂-C₆, C₂-C₄ alkynyl, and the like.

The term “hydroxyalkyl” refers to alkyl groups substituted with at least one hydroxyl (—OH) group.

The term “cycloalkyl” refers to substituted and unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments, the number of ring carbon atoms ranges from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl, cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and cryloyl groups and the like. When the group containing the carbon atom that is bonded to the carbonyl carbon atom contains a halogen, the group is termed a “haloacyl” group. An example is a trifluoroacetyl group.

The term “aryl” refers to substituted and unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, a phenyl substituted with 2, 3, 4, 5, or 6 substituents or 2-8 substituted naphthyl groups, which can be substituted with carbon or non-carbon groups such as those listed herein.

The term “heteroaryl” represents aromatic ring comprising at least one hetero atom such as N, S, O, or Se. Heteroaryl in the present disclosure may be any hetero aryl. Heteroaryl includes, but is not limited to, pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, benzimidazolinyl groups, or any combination thereof.

The term “halo” is used to describe chemical compounds which contain one or more halogen atoms, such as fluorine, chlorine, bromine, and iodine. The term “haloalkyl” group includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

The term “heterocycloalkyl” refers to a non-aromatic heterocycle where one or more of the ring-forming atoms is/are a heteroatom, such as an O, N, or S atom. Heterocycloalkyl groups can include mono- or polycyclic (e.g., having 2, 3, or 4 fused rings) ring systems as well as spirocycles. Examples of heterocycloalkyl groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of heterocycloalkyl are moieties that have one or more aromatic rings fused (i.e., having a bond in common with) to the nonaromatic heterocyclic ring, for example, phthalimidyl, naphthalimidyl, and benzo derivatives of heterocycles. A heterocycloalkyl group having one or more fused aromatic rings can be attached though either the aromatic or non-aromatic portion. Also included in the definition of hetero-cycloalkyl are moieties where one or more ring-forming atoms are substituted by 1 or 2 oxo or sulfido groups. In some embodiments, the heterocycloalkyl group has from 1 to about 20 carbon atoms, and in further embodiments from about 3 to about 20 carbon atoms. In some embodiments, the heterocycloalkyl group contains 3 to about 20, 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains O to 3 double bonds. In some embodiments, the heterocycloalkyl group contains 0 to 2 triple bonds.

It is understood that each of alkyl, cycloalkyl, alkenyl, cycloalkenyl, alkylene, and heterocycle may be optionally substituted with independently selected groups such as alkyl, haloalkyl, hydroxyalkyl, aminoalkyl, carboxylic acid and derivatives thereof, including esters, amides, and nitrites, hydroxy, alkoxy, acyloxy, amino, alky and dialky-lamino, acylamino, thio, and the like, and combinations thereof.

The terms “optionally substituted” and “optional substituents” indicate that the groups in question are either unsubstituted or substituted with one or more of the substituents specified. When the groups in question are substituted with more than one substituent, the substituents may be the same or different. The terms “independently”, “independently are,” and “independently selected from,” the groups in question may be the same or different. Certain may occur more than once in the structure and, upon such occurrence, each term shall be defined independently of the other.

The term “amine” refers to primary, secondary, and tertiary amines having, e.g., the formula N(group)₃ wherein each group can independently be H or non-H, such as alkyl, aryl, and the like. Amines include, but are not limited to, R—NH₂, for example, alkylamines, arylamines, alkylarylamines; R₂NH, wherein each R is independently selected, such as dialkylamines, diarylamines, aralkylamines, heterocyclylamines and the like; and R₃N, wherein each R is independently selected, such as trialkylamines, dialkylarylamines, alkyldiarylamines, triarylamines, and the like. The term “amine” also includes ammonium ions.

The term “amino group” refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” can be a primary, secondary, tertiary, or quaternary amino group. An “alkylamino” group includes a monoalkylamino, a dialkylamino, and a trialkylamino group.

The term “compound” as used herein, is meant to include all stereoisomers, geometric isomers, and tautomers of the structures depicted. The “optical isomers” may contain one or more chiral centers, or may otherwise be capable of existing as multiple stereoisomers. In various embodiments, the compounds are not limited to any particular stereochemical requirement, and the compounds, and compositions, methods, uses, and medicaments that include them, may be optically pure or any of a variety of stereoisomeric mixtures, including racemic and other mixtures of enantiomers, other mixtures of diastereomers, and the like. Such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. In various embodiments, the compounds are not limited to any particular geometric isomer requirement, and the compounds, may be pure or any of a variety of geometric isomer mixtures. Such mixtures of geometric isomers may include a single configuration at one or more double bonds, while including mixtures of geometry at one or more other double bonds.

The compounds can be synthesized via a Doebner-type three-component reaction involving an amine, ketone, and aldehyde. These STING antagonists can be used to treat diseases that result from over-active STING. Examples of the STING-driven diseases include, but are not limited to, inflammatory diseases, autoimmune diseases, diabetes, cancer, traumatic brain injury, and fibrosis.

In some embodiments, provided is a pharmaceutical composition comprising a compound of formula (I), or a pharmaceutically acceptable salt, hydrate, tautomer, or optical isomer thereof, and a pharmaceutically acceptable carrier, excipient, or diluent.

In some embodiments, provided is a pharmaceutical composition comprising a compound of formula (IA) or (IB), or a pharmaceutically acceptable salt, hydrate, tautomer, or optical isomer of either of the foregoing, and a pharmaceutically acceptable carrier, excipient, or diluent.

The diseases associated with overacting of STING include, but are not limited to, STING-driven inflammatory diseases, STING-driven autoimmune diseases, diabetes, cancer, traumatic brain injury, and fibrosis. In some embodiments, the diseases are inflammatory diseases.

In some embodiments, the compound of formula (I) or (IA) or (IB) or a pharmaceutically acceptable salt, hydrate, tautomer, or optical isomer thereof, can be administered by suitable routes of administration such as oral, parenteral, topical, intra-tumoral, peri-tumoral, or intra-nasal.

The terms “treat,” “treating,” “treated,” or “treatment” (with respect to a disease or condition) is an approach for obtaining beneficial or desired results including and preferably clinical results and includes, but is not limited to, one or more of the following: improving a condition associated with a disease, curing a disease, lessening severity of a disease, delaying progression of a disease, alleviating one or more symptoms associated with a disease, increasing the quality of life of one suffering from a disease, prolonging survival and/or prophylactic or preventative treatment.

The term “pharmaceutical composition” includes a therapeutically effective amount of one or more compounds for treating a STING-driven disease of patient. The composition may include other components and/or ingredients, including, but not limited to, other therapeutically active compounds and/or one or more pharmaceutically acceptable carriers, diluents, excipients, and the like. The carrier, excipient, or diluent can vary based on the particular route of administration (see, e.g., Remington's The Science and Practice of Pharmacy, 23rd ed. (2020)).

The term “therapeutic effect” refers to a beneficial local or systemic effect in animals, particularly mammals, and, more particularly humans, caused by the administration of a compound.

The term “therapeutically effective amount” means the amount of a compound that is effective to treat a disease or a disorder, such as STING-driven inflammatory diseases, STING-driven autoimmune diseases, diabetes, cancer, traumatic brain injury, and fibrosis, at a reasonable benefit/risk ratio. The therapeutically effective amount of such compound will vary depending upon the patient and the disease or disorder being treated, the weight and age of the patient, the severity of the disease or disorder, the manner of administration, and the like, which can readily be determined by one of skill in the art.

The compounds can be administered in unit dosage forms and/or compositions containing one or more pharmaceutically acceptable carriers, adjuvants, diluents, excipients, and/or vehicles, and combinations thereof. As used herein, the term “administering” and its formatives generally refer to any and all means of introducing compounds to the patient including, but not limited to, oral, intravenous, intratumoral, intramuscular, subcutaneous, transdermal, topically, and like routes of administration.

For oral administration, the compounds can be formulated readily by combining the active compound(s) with pharmaceutically acceptable carriers, excipients, or diluents well-known in the art. Such carriers, excipients, or diluents enable the compounds to be formulated as tablets, pills, powders, dragees, capsules, liquids, gels, syrups, slurries, suspensions, solutions, and the like for oral ingestion by a subject to be treated.

Useful dosages of the compounds can be determined by comparing their in vitro activity with their in vivo activity in animal models. Methods of the extrapolation of effective dosages in mice and other animals to human subjects are known in the art. Indeed, the dosage of the compounds can vary significantly depending on the condition of the subject, the age of the subject, the type of disease the subject is experiencing or at risk of experiencing, the particular compounds used, how advanced the pathology is, the route of administration of the compounds and the possibility of co-usage of other therapeutic treatments or additional drugs in combination therapies. The amount of the composition required for use in treatment (e.g., the therapeutically effective amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise.

The compositions comprising the compound(s) can be formulated in a unit dosage form, each dosage containing from about 5 to about 1,000 mg (1 g), more usually about 100 mg to about 500 mg, of the active ingredient.

In some embodiments, the compositions provided herein contain from about 5 mg to about 50 mg of the active ingredient. One having ordinary skill in the art will appreciate that this embodies compounds or compositions containing about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg of the active ingredient.

In some embodiments, the compositions provided herein contain from about 50 mg to about 500 mg of the active ingredient. One having ordinary skill in the art will appreciate that this embodies compounds or compositions containing about 50 mg to about 100 mg, about 100 mg to about 150 mg, about 150 mg to about 200 mg, about 200 mg to about 250 mg, about 250 mg to about 300 mg, about 350 mg to about 400 mg, or about 450 mg to about 500 mg of the active ingredient.

In some embodiments, the compositions provided herein contain from about 500 mg to about 1,000 mg of the active ingredient. One having ordinary skill in the art will appreciate that this embodies compounds or compositions containing about 500 mg to about 550 mg, about 550 mg to about 600 mg, about 600 mg to about 650 mg, about 650 mg to about 700 mg, about 700 mg to about 750 mg, about 750 mg to about 800 mg, about 800 mg to about 850 mg, about 850 mg to about 900 mg, about 900 mg to about 950 mg, or about 950 mg to about 1,000 mg of the active ingredient.

The active compound may be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

In some embodiments, the compound can be administered in an amount ranging from about 1 mg/kg to about 100 mg/kg. In some embodiments, the compound can be administered in an amount of about 1 mg/kg to about 20 mg/kg, about 5 mg/kg to about 50 mg/kg, about 10 mg/kg to about 40 mg/kg, about 15 mg/kg to about 45 mg/kg, about 20 mg/kg to about 60 mg/kg, or about 40 mg/kg to about 70 mg/kg. For example, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg. In some embodiments, such administration can be once-daily or twice-daily (BID) administration.

The compounds of the present disclosure can also be used in combination with other compounds or known drugs that are used as STING inhibitors. Examples of the known drugs that can be used to treat inflammatory diseases include, but are not limited to, DMXAA, FAA, H-151, C-170, C-171, CMA, GSK690693, Alpha-mangostin, Carbonyl cyanide 3-chlorophenylhydrazone (CCCP), C-178, SA-2, SN-01, and Vadimezan.

Provided is a pharmaceutical combination for treating or inhibiting STING-driven diseases in a patient in need thereof, which comprises (i) a compound of formula (I) or (IA) or (IB) or a pharmaceutically acceptable salt, hydrate, tautomer, or optical isomer of any of the foregoing, (ii) an additional therapeutic agent, and (iii) optionally at least one pharmaceutically acceptable carrier, excipient, or diluent.

The term “pharmaceutical combination” refers to a pharmaceutical therapy resulting from the mixing or combining of more than one active ingredient. For a combination, the compound of formula (I) or (IA) or (IB) or a pharmaceutically acceptable salt, hydrate, tautomer, or optical isomer thereof and at least one additional therapeutic agent can be administered to a patient simultaneously or sequentially by the same or different route of administration in a single composition or two separate compositions to achieve the desired effect. The therapeutic agent can be administered in an amount to provide its desired therapeutic effect. The effective dosage range for each therapeutic agent is well known in the art, and the therapeutic agent is administered to a patient in need thereof within such established ranges.

Provided are compounds that comprise a 3H-pyrazolo[4,3-f]quinoline moiety, which binds to STING to inhibit STING's function and is tolerable to many mammalian cell lines at concentrations of 0.5 μM or higher. Such compounds, STING activators or antagonists with low cytotoxicity profile against many mammalian cell lines, can have a higher potential to be translated for many indications than the previously described compounds that also contain the 3H-pyrazolo[4,3-f]quinoline moiety. It is not expected that such compounds would have low cytotoxicity against all cell lines but should generally have low cytotoxicity against cell lines such as raw macrophage, THP-1 and HEK-293 cell lines (24 hour incubation) at least 1 μM or such compounds should be tolerated when chronically dosed to an animal model at a concentration that leads to systemic concentrations that are adequate to engage the receptor (at least 50%) controlling the disease for at least one week.

### Identification of a Compound HSD1077 as a STING Binder

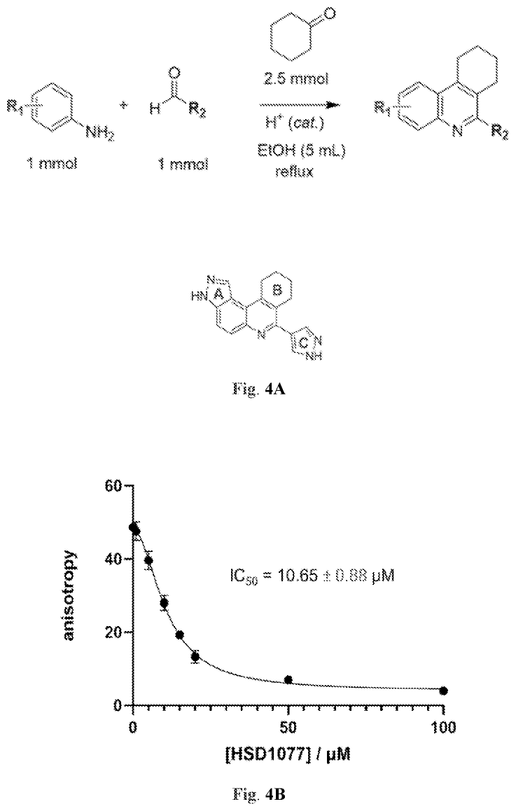

The various compounds synthesized via the Doebner-Povarov multicomponent reaction screened for STING inhibitors using a STING-based fluorescence polarization assay described in Karanja C W et al., RSC Chemical Biology, 2021, 2, 206-214, which is hereby specifically incorporated by reference for its teachings regarding same. Compound HSD1077 can compete with 50 nM of Fluorophore-c-di-GMP towards STING binding, with half maximal inhibitory concentration (IC₅₀) of 10.65 μM (see FIG. 4). Compound HSD1077, which is cell-permeable and can suppress type-1 interferon expression in both murine RAW macrophages and human THP-1 monocytes.

### SAR Evaluation of Compound HSD1077 Analogues

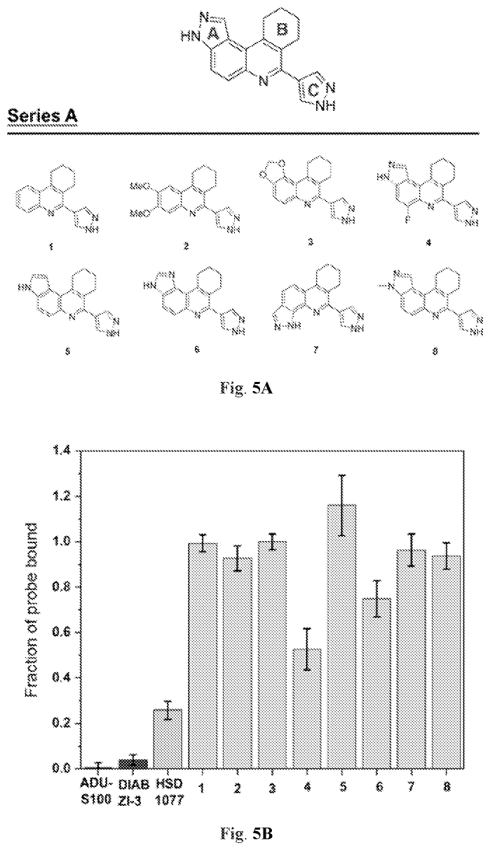

With the quinoline compound HSD1077 identified as a novel scaffold that could bind to STING, resulting in a competitive displacement of a fluorescent cyclic dinucleotide ligand, the salient motifs present in compound HSD1077 were identified that could play critical roles towards STING binding. The binding efficiency of the compound towards STING was represented in the form of a fraction of F-c-di-GMP bound (see FIG. 5), where a low fraction of bound probe (Fluorophore-c-di-GMP) suggests a potent STING binder. The importance of the 3H-pyrazolo[4,3-f]quinoline moiety, which contains ring A, towards STING binding, and generated compounds that acted as isosteres of the hit compound with vital changes around ring A was determined (FIG. 5(A)). As a preliminary screen, 20 μM of compounds and 50 nM of the probe were incubated with 10 μM of STING for 5 minutes prior to evaluation of fluorescence anisotropy. As positive controls, incubations of potent STING binding agonists ADU-S100 and DiABZI compound 3 (hereafter known as DIABZI-3) were also performed, resulting in expected high probe displacement from STING.

It was observed that the pyrazolo moiety of ring A was critical for STING binding. Deletion of the pyrazolo moiety present in compound HSD1077, as shown in analogue 1, resulted in an abrogation of STING binding. The direct replacement of the pyrazolo moiety with a dimethoxy or dioxolo moieties (compounds 2 and 3, see FIG. 5) also resulted in a significant decrease in STING binding, compared to compound HSD1077. Fluorine substitution in the 3H-pyrazolo[4,3-f]quinoline moiety at position 5 (compound 4, FIG. 5) was tolerated, with a modest reduction in STING binding compared to compound HSD1077. Changing the pyrazolo moiety in compound HSD1077 into pyrrolo (compound 5, FIG. 5) or imidazo (compound 6, FIG. 5), also led to a significant decrease in STING binding, highlighting the essentiality of the pyrazolo moiety for effective STING binding. Interestingly, another pyrazolo-containing compound 1H-pyrazolo[4,3-h]quinoline (compound 7, FIG. 5) was a poor STING binder, compared to compound HSD1077, which is a 3H-pyrazolo[4,3-f]quinoline-containing compound. A 3-methyl-3H-pyrazolo[4,3-f]quinoline compound (compound 8, FIG. 5) did not bind to STING as well as compound HSD1077, suggesting that the different functional group vectors in compound HSD1077 are important for STING binding.

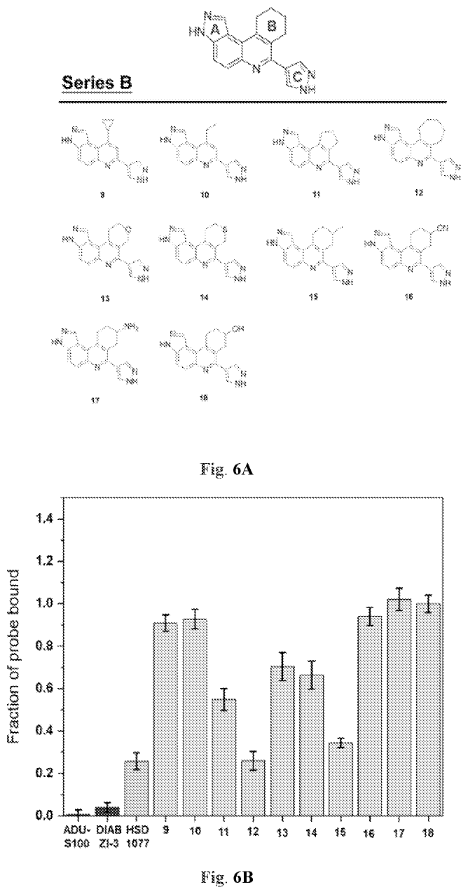

Compound HSD1077 contains a saturated six-membered ring (labeled ring B in FIG. 6A), the essentiality of this moiety was determined. The replacement of the cyclohexyl rings with an ethyl or cyclopropyl group, in the case of compound 9 and compound 10 (FIG. 6A) resulted in reduced STING binding. STING binding was restored with other ring systems (cyclopentyl or cycloheptyl rings), as observed in compound 11 and compound 12 (FIG. 6A), with compound 11 harbouring the cyclopentyl ring being slightly less effective in binding to STING in comparison to compound HSD1077. The incorporation of heteroatoms such as O and S, as seen in compounds 13 and 14 (FIG. 6A), onto the saturated cyclohexyl ring, decreased binding to STING binding. Regarding substitution to the cyclohexyl ring, the incorporation of a non-polar substituent, such as a methyl group in compound 15 (FIG. 6A), did not affect STING binding. But polar substituents such as nitrile, amine, or alcohol moieties such as compounds 16, 17 and 18 (FIG. 6A) were not well tolerated, with regards to STING binding.

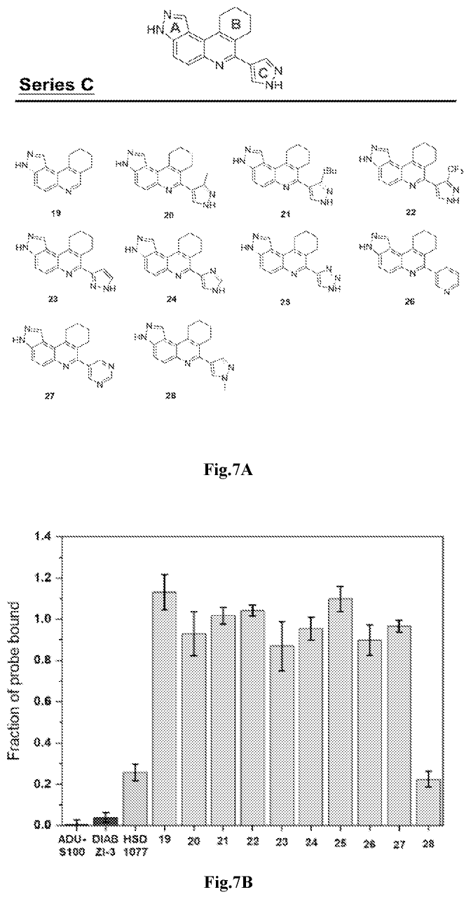

Modifications of the pyrazole ring C were explored, where the pyrazole ring was replaced with other heterocycles (FIG. 7A). The presence of the pyrazole ring was initially deemed to be critical in STING binding, as its deletion in compound 19 (FIG. 7A) led to a significant decrease in STING binding. It was noted that C-1 alkylation of the pyrazole ring, in the form of methyl, t-butyl, and trifluoromethyl substitutions, as observed in compound 20, compound 21, and compound 22 (FIG. 7A), led to reduced binding capabilities of the compounds towards STING. Nitrogen hopping within the pyrazole ring, as shown in compound 23 and compound 24 (FIG. 7A), similarly negatively impacted STING binding. The replacement of a pyrazole to a triazole, as shown in compound 25 (FIG. 7A), also led to a similar decrease in STING binding. The replacement of the pyrazole moiety with nitrogen-containing aryl moieties such as pyridine and pyrimidine groups, as in the case of compounds 26 and 27 (FIG. 7A), also negatively affected STING binding. It was interesting to note that methylation of the pyrazole ring, as seen in analogue compound 28 (FIG. 7A), generally retained STING binding, suggesting that further modifications at position-1 of the pyrazole moiety could be tolerated.

### Compound HSD1077 Attenuates Type 1 Interferon Expression in Murine RAW Macrophages

Having identified compound HSD1077 as a STING binder that could competitively displace cyclic dinucleotides, it was determined if treatment of compound HSD1077 would modulate the cGAS-STING pathway through STING binding in cells.

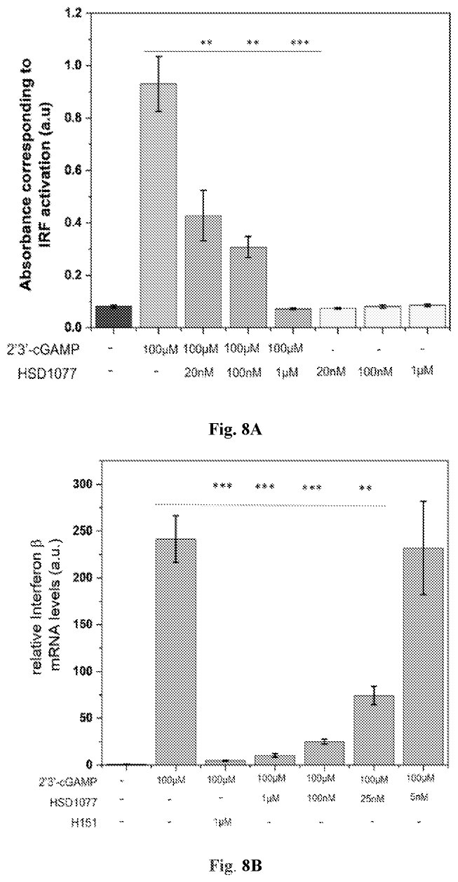

To understand possible modulatory effects of compound HSD1077 on the cGAS-STING axis, the quantification of type 1 interferon expression in-cellulo upon compound HSD1077 treatment, investigated via a commercially available murine macrophage cell line RAW ISG Blue, which express secreted embryonic alkaline phosphatase (SEAP) under the control of an interferon-stimulated gene 54 (ISG54), inducible promoter. Upon the stimulation of pathways, such as cGAS-STING, which activate type 1 interferon production, SEAP would be expressed and can be quantified using a chromogenic detection substrate Quantiblue™. Treatment of RAW ISG-blue cells with compound HSD1077 did not induce type 1 interferon production, suggesting that compound HSD1077 did not act as an agonist in the cGAS-STING pathway (FIG. 8A). Moreover, pre-treatment of compound HSD1077 (1 μM or lower) prior to STING activation with the natural ligand cGAMP was shown to result in a dose-dependent attenuation in type 1 interferon expression, suggesting that compound HSD1077 modulates the cGAS-STING axis as a STING antagonist. Excitedly, concentrations as low as 20 nM caused a noticeable decrease in interferon expression (FIG. 8).

As a secondary analysis, mRNA levels of murine Interferon-β were also quantified via quantitative PCR (qPCR), upon cGAMP stimulation on RAW macrophages with or without compound HSD1077 pre-treatment. Compound HSD1077 treatment was similarly shown to result in a dose-dependent reduction in murine Interferon-β levels as compared to DMSO-treated cell samples stimulated with cGAMP, confirming our findings through the Quantiblue assay (FIG. 8B).

### Compound HSD1077 Treatment Leads to Decreased Interferon Regulatory Factor 3 (IRF₃) Phosphorylation in RAW Macrophages

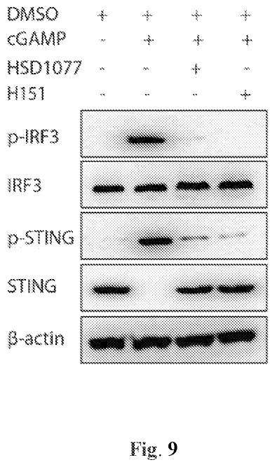

Given that the phosphorylation of IRF3 represents a highlight of the STING-TBK1-IRF3 axis, we also investigated and compared the phosphorylation of STING and IRF3 in compound HSD1077 treated and untreated samples of RAW ISG cells. The results showed that pre-treatment of compound HSD1077 at 5 μM for 4 hours prior to cGAMP stimulation showed decreased levels of phospho-STING and phospho-IRF3 when compared to DMSO+cGAMP treated samples, confirming previous studies that showed that the inhibition of the STING pathway represents a mode of action for attenuating type 1 interferon (FIG. 9).

### Compound HSD1077 Attenuates Type 1 Interferon Expression in Human THP-1 Monocytes.

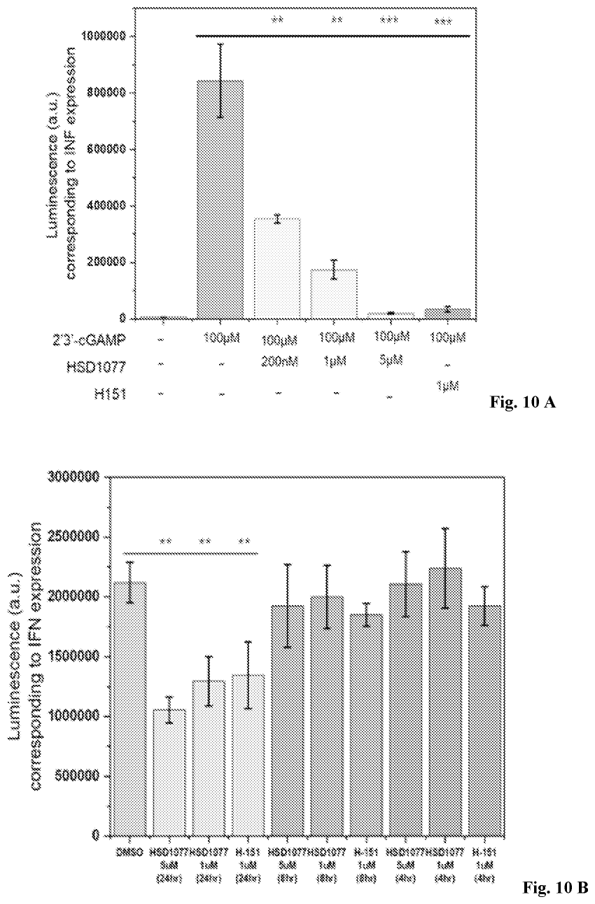

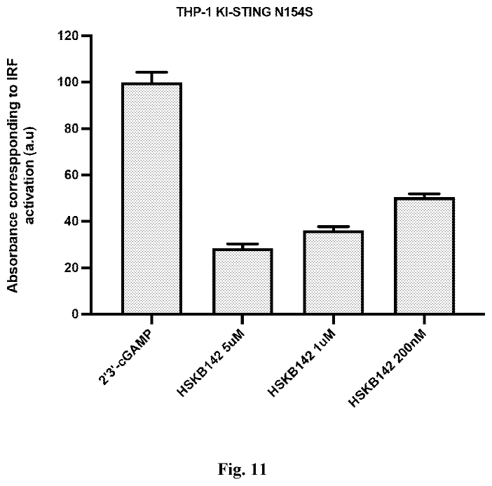

Given that murine STING (mSTING) and human STING (hSTING) are isoforms with only 61% amino acid identity in their ligand binding domain (Acta Pharm. Sin. B, 2020, 10, 2272-2298), these fundamental changes could lead to differences between mSTING and hSTING in terms of drug binding and interactions to STING. This was exemplified in the case of the mSTING agonist, which was unable to illicit agonistic effects in hSTING with a lack of bulk in the G230 region, which allowed free movement of the DMXAA molecule away from the hSTING binding site. It was investigated if compound HSD1077 would similarly result in an inhibition of type 1 interferon expression upon cGAMP stimulation in human-derived THP-1 monocytes. Using THP-1 dual cell lines (Invivogen) featuring a luciferase gene under the control of an ISG54 inducible promoter, relative quantifications of type 1 interferon expression could be achieved through luciferase detection upon cGAS-STING pathway induction. The results showed that pre-treatment of THP-1 monocytes with compound prior to cGAMP stimulation showed reduced type 1 interferon expression in a dose-dependent manner similar to results obtained with murine RAW macrophage pre-treatment (see FIG. 10A). This indicates that compound HSD1077 can attenuate type 1 interferon in both murine and human cell lines and can target both mSTING and hSTING.

In autoimmune diseases featuring chronic inflammation, such as SAVI, plausible contributory factors include the expression of constitutionally active STING mutants, such as N154S or V155M STING isoforms. This results in chronically upregulated expression of type 1 interferons without the need for ligand activation, leading to undesired inflammatory symptoms. Hence, it was identified if compound HSD1077 could reduce type 1 interferon expression in cell models expressing constitutively active STING isoforms. Upon treatment of compound HSD1077 to THP-1 dual cells expressing a knock-in constitutionally active STING (N154S) isoform and detection of expressed luciferase corresponding to, it was observed upon a 24-hour treatment of THP-1 dual monocytes expressing a knock-in STING (N154S) phenotype with compound HSD1077 and quantification of Lucia luciferase expressed using the luminescence reagent QuantiLuc™, a modest 42% reduction in luminescence was observed when compared to DMSO control (FIG. 10B), suggesting that compound HSD1077 was indeed able to modulate type 1 interferon expression through STING inhibition with extended compound HSD1077 treatment.

### EXAMPLES

The following examples serve to illustrate the present disclosure. The examples are not intended to limit the scope of the claimed invention in any way.

### Assays:

Expression and Affinity Purification of hSTING

The cloned plasmid harbouring hSTING gene (PET28a, SUMO) transformed into E. coli Rosetta™2 (pLysS) cells. Kanamycin (50 μg/mL) and chloramphenicol (32 μg/mL) was employed as selection factors. A single colony was picked and grown in 10 mL of LB media with selection factors incorporated and incubated at 37° C. overnight. The culture was then inoculated into 1 L of terrific broth with kanamycin and chloramphenicol supplementation and grown at 37° C. till the exponential phase was reached, where OD₆₀₀=0.6. Protein expression was induced through the addition of isopropyl-b-_{D}-thiogalactopyranoside to a 1 mM final concentration. The culture was incubated at 25° C. for 18 hours. Obtained cells were pelleted via centrifugation for 30 minutes at 5000 rpm. The obtained bacteria pellet was then resuspended in 25 mL of lysis buffer (50 mM Na₃PO₄, 300 mM NaCl, 20 mM imidazole, 5 mM 2-mercaptoethanol, 10% glycerol, and 1× complete protein inhibitor cocktail). Cells were lysed through sonication and the lysate was centrifuged at 22000 rpm for 25 minutes, followed by collection of the supernatant. The supernatant containing hSTING was purified by passing through a HisTrap-HP column and pure hSTING eluted with elution buffer containing 50 mM Na₃PO₄ (pH=7.4), 300 mM NaCl, 300 mM Imidazole, 5 mM 2-mercaptoethanol, 10% glycerol. The purified hSTING was dialyzed for 24 hours in a dialysis buffer containing 50 mM Na₃PO₄ (pH=7.4), 300 mM NaCl, 5 mM β-mercaptoethanol and 10% glycerol for the removal of imidazole, and quantified via absorbance measurements at 2=280 nm with ε=47955 M⁻¹ cm⁻¹

STING based Fluorescence Polarization Assay

50 nM of 2′-Fluo-AHC-c-diGMP (Biolog) was incubated with 10 μM hSTING and 20 μM of screening compounds in dimethyl sulfoxide (DMSO) for 5 minutes in 1× Phosphate buffered saline at room temperature. Fluorescence polarization (λ_{ex/em}=485/528 nm) was then quantified via Biotek Cytation 5 multi-mode reader, with anisotropy calculated using Gen5 microplate reader and imaging software. Anisotropy was normalized via equating measurements with 0 μM hSTING to zero. Experiments were performed in triplicates using 384 Greiner-Bio 384 fluorometric plate (flat plate). Anisotropy was converted to a fraction bound by F-c-di-GMP using the following equation:

$${F}_{b o u n d} = \frac{r - {r}_{f r e e}}{( {r}_{b o u n d} - r ) Q + ( r - {r}_{free} )}$$

wherein r refers to the anisotropy value at 10 μM hSTING concentration upon compound addition, r_{free} refers to the anisotropy value of unbound fluorophore, and Q refers to the ratio of fluorescence intensities between bound and free fluorophore. Anisotropy changes with increasing concentrations of compound HSD1077 was plotted, with a 4-parameter dose-response fit applied to obtain the reported IC₅₀ via GraphPad Prism (San Diego, CA, USA).

Cell Viability of RAW ISG Blue reporter cells upon compound HSD1077 treatment RAW ISG Blue macrophage reporter cells (Invivogen) were cultured in DMEM containing 10% heat inactivated foetal bovine serum and 1× Penicillin/Streptomycin at 37° C., with 5% CO₂. 2×10³ cells were seeded in 96 well plates and incubated for 24 hours to allow for adherence. Cells were then treated with increasing concentrations of compound HSD 1077 for 24 hours. After which, CellTiter-Blue cell viability assay reagent (Promega) was added based on manufacturer recommendations and incubated for 3 hours. Fluorescence (λ_{ex/em}=560/590 nm) of each well was quantified via Biotek Cytation 5 multi-mode reader. Experiments were performed in biological triplicates, with data reported as the mean and standard deviation of 3 data points. Readings from cell samples treated with DMSO was normalized to 100%.

### Detection of IRF Activation in RAW ISG Blue Reporter Cells

RAW ISG Blue macrophage reporter cells (Invivogen) were cultured in DMEM containing 10% heat inactivated foetal bovine serum and 1× Penicillin/Streptomycin at 37° C., with 5% CO₂. 1×10⁵ cells were seeded in 96 well plates and incubated for 24 hours to allow for adherence. After which, cells were pre-treated with the drug compound for 2 hours, followed by the addition of 100 μM of 2′-3′ cGAMP for induction of the cGAS-STING pathway and incubated for 24 hours. After which, media from each respective well was collected for estimation of IRF activation via SEAP colorimetric assay via QUANTI-blue reagent (Invivogen) as per manufacturer protocols, using a clear 96 well flat bottom plate. 20 μL of the media obtained in each individual well was incubated with 180 μL of QUANTI-Blue™ Solution for 6 hours at 37° C. Absorbance (630 nm) of each well was quantified via Biotek Cytation 5 multi-mode reader. Experiments were performed in biological triplicates, with data reported as the mean and standard deviation of 3 data points.

### Detection of IRFactivation in THP-1 Dual Reporter Cells

THP-1 dual or THP-1 (STING N154S) dual reporter cells (Invivogen) were cultured in RPMI media containing 10% heat inactivated foetal bovine serum and 1× penicillin/streptomycin in 37° C., 5% CO₂. 1×10⁵ cells were seeded in 96 well plates and incubated for 24 hours. After which, cells were pre-treated with the drug compound for 1 hour, followed by 100 μM of 2′-3′ cGAMP for induction of the cGAS-STING pathway, and incubated for 24 hours. After which, cell suspension from each respective well was collected and for relative quantification of IRF activation via Lucia luciferase based luminescent assay via QUANTI-Luc™ reagent (Invivogen). 10 μL of the cell suspension obtained in each individual well was added to 50 μL of QUANTI-Luc™ Solution. Endpoint luminescence measurements were then obtained via Biotek Cytation 5 multi-mode reader with a 4 second start time and 0.1 second reading time. Experiments were performed in biological triplicates, with data reported as the mean and standard deviation of 3 data points.

Evaluation of Interferon-β mRNA Levels in RAW Cells Via qPCR

RAW ISG Blue macrophages were cultured in DMEM containing 10% heat inactivated foetal bovine serum. 1×10⁶ cells were seeded in 6 well plates and incubated for 24 hours to allow for adherence. After 24 hours, cells were pre-treated with compound HSD1077 or compound H-151 for 6 hours, followed by 100 μM of 2′-3′ cGAMP for 3 more hours. Cells were subsequently harvested, and RNA extraction was performed via the use of TRIzol reagent (Thermo-fisher). 1 μg of RNA harvested from each sample was used for cDNA synthesis using random hexamers, dNTPs, and superscript II reverse transcriptase (Thermo-fisher). For the qPCR analysis, 2× Quanti-tect SYBR green master-mix (Qiagen) was used as per manufacturer recommendations. The forward and reverse primers for the relative quantification of Interferon-β and β-actin were used as reported in Wiser C, Science Reports, 2020, 10, 1-11 and Wang M, ACS Chemical Biology, 2021, 16, 1663-1670, which are hereby specifically incorporated by reference for its teachings regarding the same.

Evaluation of Phosphorylated Interferon Regulatory Factor 3 (pIRF₃) Levels in RAW Cells Via Western Blotting

0.5-1×10⁶ Raw ISG cells were seeded in 6-well plates. Post 24 h, cells were treated with compound HSD1077 or H151 for 4h, which was followed by 100 μM 2′,3′-cGAMP treatment for 3h. Cells were then harvested in RIPA lysis buffer (50 mM Tris (pH 7.4), 150 mM NaCl, 1% Triton X-100, 0.1% sodium dodecyl sulfate (SDS), 0.5% sodium deoxycholate) with protease inhibitor cocktail (Roche) and 1 mM phenylmethylsulfonyl fluoride. Post sonication, cells were centrifuged at 14000×g for 15 mins, and the supernatant was collected. Protein quantification was done using Pierce Rapid Gold BCA Protein Assay Kit (Thermo-Fisher). Next, 10% SDS polyacrylamide gel electrophoresis was performed, and then proteins were transferred to a polyvinylidene difluoride (PVDF) membrane. The membrane was probed with pSTING (CST #50907), STING (CST, #13647), pTBK1 (CST #5483), TBK1 (CST #3013) pIRF₃ (CST #E7J8G), IRF3 (CST #4302) and β-actin (CST #8457) antibodies overnight at 2-8° C. After overnight incubation, the membrane was further incubated with the corresponding horseradish peroxidase (HRP)-conjugated secondary antibodies at 37° C. for 2 h. SuperSignal West Pico PLUS Chemiluminescent Substrate was used for signal detection on Azure 300 imaging system.

Solvents and reagents were obtained from commercial sources and utilized without further purification. ¹H and ¹³C NMR spectra presented were obtained in methanol-d₄ or DMSO-d₆ using a Bruker AV500 (500 MHz) or AV800 (800 MHz) spectrometer with tetramethylsilane as an internal standard. ¹H NMR data were reported as shown: chemical shift (8 ppm) (multiplicity, coupling constant (Hz), integration). Chemical shifts were reported in downfield order in parts per million (8 ppm). Multiplicities are reported as follows: s=singlet, brs=broad singlet, d=doublet, t=triplet, q=quartet, m=multiplet, or combinations thereof. Electron spray ionization (ESI) technique and TOF mass analysis were used to record high-resolution mass spectra (HRMS). All the synthesized compounds were characterized using ¹H, ¹³C, and HRMS.

The quinoline compounds were prepared using the procedure, which is well known in the art (Dayal et al., European Journal of Medicinal Chemistry, 2019, 180, 449-456), which is hereby specifically incorporated by reference for its teachings regarding the same.

### General Procedure

In a screw-capped glass vial (20 mL), the corresponding amine (1 mmol) and aldehyde (1 mmol) were refluxed in absolute ethanol (5 mL) for 2 hours. After that, the reaction mixture was cooled to room temperature, followed by the addition of the corresponding ketone (2.5 mmol) and a catalytic amount of conc. hydrogen chloride. Further reaction was allowed to reflux for an additional 6 to 12 hours. Upon completion, the reaction mixture was concentrated and purified using silica gel column chromatography (Hexanes: Ethyl acetate 50:50 to 0:100) or Ethyl Acetate/methanol (99:01 to 80:20).

### Example 1

### 7-(1H-pyrazol-4-yl)-8,9,10,11-tetrahydro-3H-pyrazolo[4,3-a]phenanthridine (compound HSD1077)

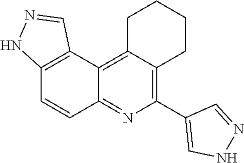 [SMILES: c1n[nH]cc1-c1nc2ccc3[nH]ncc3c2c2c1CCCC2]

The compound was prepared using the procedure well known in the art (Dayal et al., Future Medicinal Chemistry 2018, 10, 823-835), which is hereby specifically incorporated by reference for its teachings regarding the same.

### Example 2

### 6-(1H-pyrazol-4-yl)-7,8,9,10-tetrahydrophenanthridine (Compound 1)

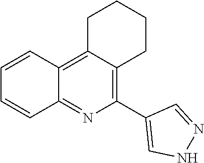 [SMILES: c1ccc2c3c(c(-c4cn[nH]c4)nc2c1)CCCC3]

The compound was prepared using the general procedure. Yellow solid (99 mg, 40%).

¹H NMR (500 MHz, DMSO-d₆) δ 8.22 (brs, 1H), 8.08 (brs, 1H) 7.96 (d, J=4.2 Hz, 1H), 7.89 (d, J=3.9 Hz, 1H), 7.26 (td, J=7.1, 1.2 Hz, 1H), 7.50 (td, J=5.7, 1.2 Hz, 1H), 3.13 (t, J=6.3 Hz, 2H), 2.94 (t, J=6.0 Hz, 2H), 1.87-1.90 (m, 2H), 1.78-1.81 (m, 2H);

¹³C NMR (125 MHz, DMSO-d₆): 153.06, 145.73, 141.85, 140.20, 129.46, 128.67, 128.24, 126.22, 126.02, 123.19, 121.61, 28.40, 25.75, 22.86, 22.01.

HRMS (ESI) m/z calcd for C₁₆H₁₅N₃ [M+H]⁺ 250.1344, found 250.1343.

### Example 3

### 2,3-Dimethoxy-6-(1H-pyrazol-4-yl)-7,8,9,10-tetrahydrophenanthridine (Compound 2)

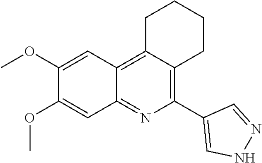 [SMILES: COc1cc2nc(-c3cn[nH]c3)c3c(c2cc1OC)CCCC3]

Off-white solid (133 mg, 43%).

¹H NMR (500 MHz, DMSO-d₆): δ 8.16 (s, 1H), 7.99 (s, 1H), 7.25 (s, 1H), 7.17 (s, 1H), 3.90 (s, 3H), 3.88 (s, 3H), 3.06 (t, J=6.4 Hz, 2H), 2.89 (t, J=6.0 Hz, 2H), 1.85-1.90 (m, 2H), 1.77-1.80 (m, 2H); ¹³C NMR (125 MHz, DMSO-d₆): 151.46, 150.46, 149.18, 142.51, 140.87, 140.40, 126.09, 121.83, 121.25, 108.41, 101.66, 55.94, 55.37, 28.26, 26.05, 23.05, 22.16.

HRMS (ESI) m/z calcd for C₁₈H₁₉N₃O₂ [M+H]⁺ 310.1555, found 310.1557.

### Example 4

### 7-(1H-pyrazol-4-yl)-8,9,10,11-tetrahydro-[1,3]dioxolo[4,5-a]phenanthridine (Compound 3)

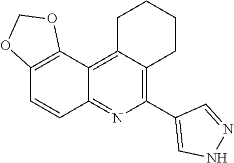 [SMILES: c1n[nH]cc1-c1nc2ccc3c(c2c2c1CCCC2)OCO3]

Brown solid. (58 mg, 20%).

¹H NMR (500 MHz, DMSO-d₆) δ 8.15 (brs, 1H), 7.98 (brs, 1H), 7.30 (s, 1H), 7.22 (s, 1H), 6.15 (s, 2H), 2.99 (t, J=5.9 Hz, 2H), 2.87 (t, J=5.5 Hz, 2H), 1.83-1.86 (m, 2H), 1.77-1.78 (m, 2H); ¹³C NMR (125 MHz, DMSO-d₆): 150.61, 149.58, 147.48, 143.65, 141.04, 139.81, 129.39, 126.41, 122.72, 121.63, 105.62, 102.08, 99.10, 28.17, 26.22, 22.93, 22.17.

HRMS (ESI) m/z calcd for C₁₇H₁₅N₃O₂ [M+H]⁺ 294.1242, found 294.1244.

### Example 5

### 7-(1H-pyrazol-4-yl)-8,9,10,11-tetrahydro-3H-pyrrolo[3,2-a]phenanthridine (Compound 4)

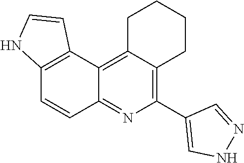 [SMILES: c1cc2c(ccc3nc(-c4cn[nH]c4)c4c(c32)CCCC4)[nH]1]

Yellow solid (147 mg, 51%).

¹H NMR (500 MHz, DMSO-d₆) δ 12.38 (s, 1H), 8.37 (s, 2H), 8.13-7.93 (m, 2H), 7.72-7.54 (m, 1H), 7.15 (s, 1H), 3.39 (t, J=6.4 Hz, 2H), 2.97 (t, J=6.2 Hz, 2H), 2.02-1.93 (m, 2H), 1.82-1.76 (m, 2H); ¹³C NMR (200 MHz, DMSO-d₆) δ 150.92, 144.49, 140.42, 135.86, 134.05, 133.38, 131.51, 128.59, 126.08, 122.16, 119.80, 115.93, 114.52, 106.42, 31.00, 28.12, 21.98, 21.78.

HRMS (ESI) m/z calcd for C₁₈H₁₇N₄ [M+H]⁺ 289.1453, found 289.1451.

### Example 6

### 7-(1H-pyrazol-4-yl)-8,9,10,11-tetrahydro-3H-pyrazolo[4,3-a]phenanthridin-9-amine (Compound 5)

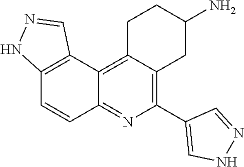 [SMILES: NC1CCc2c(c(-c3cn[nH]c3)nc3ccc4[nH]ncc4c23)C1]

Light brown solid (80 mg, 27%).

¹H NMR (800 MHz, DMSO-d₆) δ 8.59-8.47 (m, 3H), 8.27 (d, J=9.2 Hz, 1H), 8.07 (d, J=9.3 Hz, 1H), 3.78-3.59 (m, 2H), 3.03-2.90 (m, 2H), 1.99-1.87 (m, 2H), 1.83-1.75 (m, 2H); ¹³C NMR (200 MHz, DMSO-d₆) δ 152.88, 145.51, 142.11, 140.01, 136.34, 134.55, 132.16, 129.47, 119.54, 116.19, 112.73, 31.20, 27.90, 21.68, 21.45.

HRMS (ESI) m/z calcd for C₁₇H₁₆N₅ [M+H]⁺ 290.1405, found 290.1405.

### Example 7

### 5-(1H-pyrazol-4-yl)-6,7,8,9-tetrahydro-3H-pyrazolo[4,3-c]phenanthridine (Compound 6)

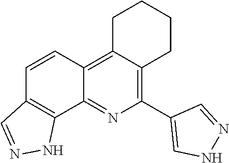 [SMILES: c1n[nH]cc1-c1nc2c(ccc3cn[nH]c32)c2c1CCCC2]

Pale brown solid, (84 mg, 29%).

¹H NMR (500 MHz, DMSO-d₆): δ 8.54 (s, 1H), 8.23 (brs, 2H), 7.86 (d, J=4.2 Hz, 1H), 7.63 (d, J=4.5 Hz, 1H) 2.97 (t, J=7.7 Hz, 2H), 2.96 (t, J=5.2 Hz, 2H), 1.90 (d, J=2.5 Hz, 2H), 1.82 (d, J=2.5 Hz, 2H); ¹³C NMR (125 MHz, DMSO-d₆): 152.12, 142.89, 141.10, 135.54, 133.91, 126.39, 122.26, 121.94, 121.03, 119.96, 111.37, 28.34, 26.56, 22.90, 22.24. HRMS (ESI) m/z calcd for C₁₇H₁₅N₅ [M+H]⁺ 304.1562, found 304.1565.

### Example 8

### 3-Methyl-7-(1H-pyrazol-4-yl)-8,9,10,11-tetrahydro-3H-pyrazolo[4,3-a]phenanthridine (Compound 7)

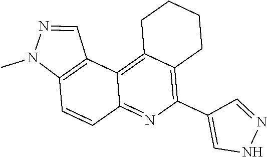 [SMILES: Cn1ncc2c3c4c(c(-c5cn[nH]c5)nc3ccc21)CCCC4]

Off-white solid. (81 mg, 27%).

¹H NMR (500 MHz, DMSO-d₆): δ8.45 (s, 1H), 8.22 (brs, 1H), 8.06 (brs, 1H), 7.93 (d, J=4.7 Hz, 1H), 7.83 (d, J=4.6 Hz, 1H), 4.15 (s, 3H), 2.99 (t, J=6.0 Hz, 2H), 2.48 (t, J=1.6 Hz, 2H), 1.97 (quin, J=2.3 Hz, 2H), 1.84 (quin, J=2.4 Hz, 2H); ¹³C NMR (125 MHz, DMSO-d₆): δ149.95, 143.57, 142.13, 140.96, 139.98, 138.04, 134.85, 129.57, 129.12, 121.60, 120.82, 117.30, 113.68, 36.24, 29.82, 22.42.

HRMS (ESI) m/z calcd for C₁₈H₁₇N₅ [M+H]⁺ 304.1562, found 304.1565.

### Example 9

### 9-Cyclopropyl-7-(1H-pyrazol-4-yl)-3H-pyrazolo[4,3-f]quinoline (Compound 8)

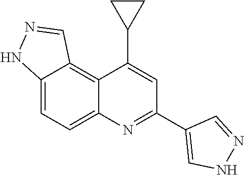 [SMILES: c1n[nH]cc1-c1cc(C2CC2)c2c(ccc3[nH]ncc32)n1]

Brown solid (66 mg, 24%).

¹H NMR (500 MHz, Methanol-d₄): δ 8.79 (s, 1H), 8.31 (s, 2H), 7.86-7.94 (m, 2H), 7.67 (s, 1H), 1.28-1.35 (m, 2H), 1.00-1.02 (m, 2H); ¹³C NMR (125 MHz, Methanol-d₄): 149.76, 149.20, 143.05, 128.51, 123.68, 122.54, 119.50, 118.99, 117.33, 114.66, 110.48, 99.65, 39.01, 29.34, 15.86.

HRMS (EST) m/z calcd for C₁₆H₁₃N₅ [M+H]⁺ 276.1249, found 276.1248.

### Example 10

### 9-Ethyl-7-(1H-pyrazol-4-yl)-3H-pyrazolo[4,3-f]quinoline (Compound 9)

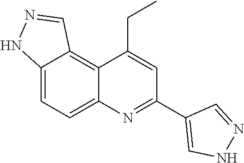 [SMILES: CCc1cc(-c2cn[nH]c2)nc2ccc3[nH]ncc3c12]

White solid. (81 mg, 31%).

¹H NMR (500 MHz, Methanol-d₄) δ 8.53 (s, 1H), 8.33 (s, 2H), 7.98 (d, J=4.5 Hz, 1H), 6.98 (d, 4.5 Hz, 1H), 7.82 (s, 1H), 3.38 (d, J=7.5 Hz, 2H), 1.53 (t, J=7.5 Hz, 3H); ¹³C NMR (125 MHz, Methanol-d₄) δ 150.52, 149.76, 146.21, 138.19, 135.16, 128.74, 126.94, 122.61, 120.58, 118.69, 116.18, 114.46, 27.79, 12.00.

HRMS (ESI) m/z calcd for C₁₅H₁₃N₅ [M+H]⁺ 264.1249, found 264.1248.

### Example 11

### 7-(1H-pyrazol-4-yl)-3,8,9,10-tetrahydrocyclopenta[c]pyrazolo[4,3-f]quinoline (Compound 10)

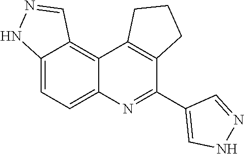 [SMILES: c1n[nH]cc1-c1nc2ccc3[nH]ncc3c2c2c1CCC2]

Off-white solid (115 mg, 42%).

¹H NMR (500 MHz, DMSO-d₆): δ 8.40 (s, 1H), 8.28 (brs, 1H), 8.15 (brs, 1H), 7.79-7.85 (m, 2H), 3.47 (2H, brs), 3.27 (t, J=7.4 Hz, 2H), 2.32 (quin, J=7.6 Hz, 2H); ¹³C NMR (125 MHz, DMSO-d₆): 149.00, 146.75, 144.64, 138.80, 137.96, 134.86, 129.01, 128.48, 122.38, 118.35, 116.99, 114.36, 33.41, 32.78, 24.09.

HRMS (ESI) m/z calcd for C₁₆H₁₃N₅ [M+H]⁺ 276.1249, found 276.1252.

### Example 12

### 7-(1H-pyrazol-4-yl)-3,8,9,10,11,12-hexahydrocyclohepta[c]pyrazolo[4,3-f]quinoline (Compound 11)

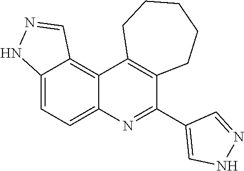 [SMILES: c1n[nH]cc1-c1nc2ccc3[nH]ncc3c2c2c1CCCCC2]

Brown solid (39 mg, 15%).

¹H NMR (Methanol-d₄) δ7.96 (s, 1H), 7.56 (s, 2H), 7.19 (d, J=4.35 Hz, 1H) 6.86 (d, J=4.4 Hz, 1H), 4.04 (d, J=4.0 Hz, 1H), 3.03 (d, J=4.3 Hz, 1H), 2.85 (d, J=1.6 Hz, 1H), 2.23 (t, J=8.3 Hz, 1H), 1.72-1.74 (m, 2H), 1.60-1.65 (m, 2H), 1.36-1.38 (m, 1H), 1.10 (d, J=5.0 Hz, 1H); ¹³C NMR (125 MHz, Methanol-d₄): δ149.73, 143.70, 142.13, 139.91, 138.49, 136.15, 129.62, 129.17, 128.90, 121.64, 121.00, 116.55, 114.16, 49.06, 29.85, 28.66, 22.64, 22.48.

HRMS (ESI) m/z calcd for C₁₈H₁₇N₅ [M+H]⁺ 304.1562, found 304.1563.

### Example 13

### 7-(1H-pyrazol-4-yl)-3,8,10,11-tetrahydropyrano[3,4-c]pyrazolo[4,3-f]quinoline (Compound 12)

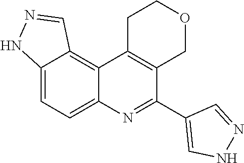 [SMILES: c1n[nH]cc1-c1nc2ccc3[nH]ncc3c2c2c1COCC2]

White solid. (75 mg, 26%).

¹H NMR (500 MHz, Methanol-d₄): δ 8.55 (s, 1H), 8.07 (s, 2H), 7.84-7.91 (m, 2H), 4.99 (s, 2H), 4.22 (t, J=4.4 Hz, 2H) 3.41 (brs, 2H); ¹³C NMR (125 MHz, Methanol-d₄): δ 149.87, 146.49, 145.79, 143.68, 139.77, 136.12, 132.18, 128.68, 128.06, 127.39, 120.99, 119.91, 76.08, 66.82, 64.04, 54.83, 31.66, 29.03, 29.00, 28.61.

HRMS (ESI) m/z calcd for C₁₆H₁₃N₅O [M+H]⁺ 292.1198, found 292.1200.

### Example 14

### 7-(1H-pyrazol-4-yl)-3,8,10,11-tetrahydropyrazolo[4,3-f]thiopyrano[3,4-c]quinoline (Compound 13)

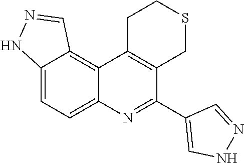 [SMILES: c1n[nH]cc1-c1nc2ccc3[nH]ncc3c2c2c1CSCC2]

Pale brown solid (120 mg, 38%).

¹H NMR (500 MHz, DMSO-d₆): δ 8.56 (s, 1H), 8.22 (brs, 1H), 8.11 (brs, 1H), 7.81-7.87 (m, 2H), 4.11 (s, 2H), 3.59 (brs, 2H), 3.13 (t, J=6.0 Hz, 2H); ¹³C NMR (125 MHz, DMSO-d₆): 148.93, 144.06, 141.91, 138.64, 136.30, 129.61, 126.76, 121.38, 121.01, 116.25, 114.86, 31.44, 29.04, 25.66.

HRMS (ESI) m/z calcd for C₁₆H₁₃N₅S [M+H]w⁺ 308.0969, found 308.0974

### Example 15

### 9-Methyl-7-(1H-pyrazol-4-yl)-8,9,10,11-tetrahydro-3H-pyrazolo[4,3-a]phenanthridine (Compound 14)

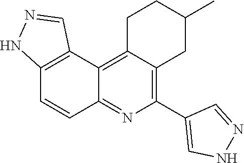 [SMILES: CC1CCc2c(c(-c3cn[nH]c3)nc3ccc4[nH]ncc4c23)C1]

Yellow solid (118 mg, 39%).

¹H NMR (500 MHz, DMSO-d₆): δ 8.49 (s, 1H), 8.17 (brs, 1H), 8.05 (brs, 1H), 7.81 (s, 2H), 3.21-3.27 (m, 1H), 3.00 (dd, J=8.3 Hz, 3.7 Hz, 1H), 2.63-2.68 (m, 1H), 2.09 (brs, 1H), 1.82 (brs, 1H), 1.55 (hept, J=6.0 Hz, 1H), 1.11 (d, J=6.5 Hz, 3H); ¹³C NMR (125 MHz, DMSO-d₆): δ149.72, 143.71, 141.80, 140.03, 138.49, 136.14, 129.56, 128.63, 121.63, 120.82, 116.58, 114.19, 36.89, 30.43, 29.79, 28.58, 22.08.

HRMS (ESI) m/z calcd for C₁₈H₁₇N₅ [M+H]⁺ 304.1562, found 304.1560.

### Example 16

### 7-(1H-pyrazol-4-yl)-8,9,10,11-tetrahydro-3H-pyrrolo[3,4-a]phenanthridine-9-carbonitrile (Compound 15)

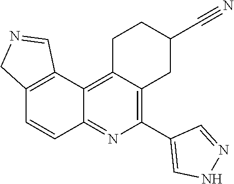 [SMILES: N#CC1CCc2c(c(-c3cn[nH]c3)nc3ccc4c(c23)C=NC4)C1]

Pale brown solid. (116 mg, 37%).

¹H NMR (500 MHz, Methanol-d₄): δ 8.51 (s, 1H), 8.22 (brs, 1H), 8.05 (brs, 1H), 7.82-7.88 (m, 2H), 5.73 (s, 1H), 4.92 (s, 2H), 3.94 (quin, J=6.1 Hz, 2H), 2.16 (s, 2H); ¹³C NMR (125 MHz, Methanol-d₄): δ 169.01, 147.93, 147.59, 144.13, 140.04, 138.53, 135.87, 129.43, 126.22, 120.82, 120.56, 116.36, 115.02, 55.36, 47.28, 29.71, 21.46.

HRMS (ESI) m/z calcd for C₁₈H₁₄N₆ [M+H]⁺ 315.1358, found 315.1358.

### Example 17

### 2-(7-(1H-pyrazol-4-yl)-8,9,10,11-tetrahydro-3H-pyrazolo[4,3-a]phenanthridin-9-yl)isoindoline-1,3-dione (Compound 16)

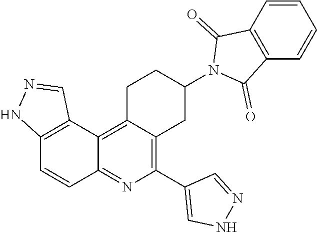 [SMILES: O=C1c2ccccc2C(=O)N1C1CCc2c(c(-c3cn[nH]c3)nc3ccc4[nH]ncc4c23)C1]

Off-white solid (191 mg, 44%).

¹H NMR (800 MHz, DMSO-d₆) δ 8.78-8.66 (m, 1H), 8.43-8.21 (m, 3H), 8.16 (s, 1H), 7.96-7.77 (m, 4H), 4.65-4.50 (m, 1H), 3.78-3.65 (m, 2H), 3.60 (s, 1H), 3.24 (dd, J=16.3, 5.1 Hz, 1H), 2.86-2.66 (m, 1H), 2.43-2.26 (m, 1H); ¹³C NMR (200 MHz, DMSO-d₆) δ 168.37, 145.76, 138.71, 136.17, 134.70, 132.15, 128.66, 123.46, 123.34, 121.47, 118.25, 115.45, 46.51, 31.19, 30.76, 25.36.

HRMS (ESI) m/z calcd for C₂₅H₁₉N₆O₂ [M+H]⁺ 435.1569, found 435.1565.

### Example 18

### 7-(1H-pyrazol-4-yl)-8,9,10,11-tetrahydro-3H-imidazo[4,5-a]phenanthridine (Compound 17)

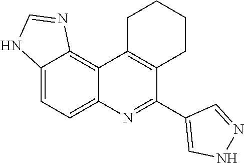 [SMILES: c1nc2c(ccc3nc(-c4cn[nH]c4)c4c(c32)CCCC4)[nH]1]

Synthesized from compound 16. In a 20 mL reaction vial, compound 16 (150 mg, 0.34 mmol) was dissolved in methanol (4 mL), followed by the addition of hydrazine monohydrate (0.5 mmol). The reaction was refluxed for 5 hours. After completion, the reaction was concentrated to dryness and purified via silica gel chromatography to get the desired deprotected compound. Off-white solid (79 mg, 75%).

¹H NMR (800 MHz, DMSO-d₆) δ 8.67-8.51 (m, 3H), 8.22 (s, 2H), 7.93 (s, 2H), 3.57 (s, 1H), 3.52-3.40 (m, 2H), 3.36-3.29 (m, 1H), 3.23-3.11 (m, 1H), 2.49-2.40 (m, 1H), 2.12-2.01 (m, 1H); ¹³C NMR (200 MHz, DMSO-d₆) δ 147.96, 146.45, 143.58, 141.84, 140.30, 138.61, 136.00, 131.28, 125.57, 120.64, 118.77, 115.85, 46.27, 32.26, 28.23, 25.82. HRMS (ESI) m/z calcd for C₁₇H₁₇N₆ [M+H]⁺ 305.1514, found 305.1512.

### Example 19

### 7-(1H-pyrazol-4-yl)-8,9,10,11-tetrahydro-3H-pyrazolo[4,3-a]phenanthridin-9-ol (Compound 18)

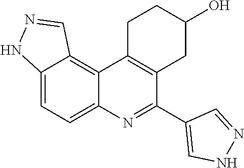 [SMILES: OC1CCc2c(c(-c3cn[nH]c3)nc3ccc4[nH]ncc4c23)C1]

Off-white solid (100 mg, 26%).

¹H NMR (500 MHz, DMSO-d₆) δ 8.54 (s, 1H), 8.12 (bs, 2H), 7.88-7.68 (m, 2H), 4.92 (d, J=3.3 Hz, 1H), 4.06 (td, J=7.2, 3.6 Hz, 1H), 3.47-3.26 (m, 2H), 3.19 (td, J=15.5, 14.4, 3.7 Hz, 1H), 2.91 (dd, J=16.5, 7.1 Hz, 1H), 2.14 (q, J=6.5 Hz, 1H), 1.93 (dq, J=12.7, 6.7, 5.9 Hz, 1H); ¹³C NMR (125 MHz, DMSO) δ 149.91, 143.78, 141.67, 138.53, 136.18, 129.47, 127.02, 121.51, 120.60, 116.53, 114.35, 64.71, 37.54, 30.26, 27.71.

HRMS (ESI) m/z calcd for C₁₇H₁₆N₅O [M+H]⁺ 306.1354, found 306.1356.

### Example 20

### 7-(1H-pyrazol-3-yl)-8,9,10,11-tetrahydro-3H-pyrazolo[4,3-a]phenanthridine (Compound 19)

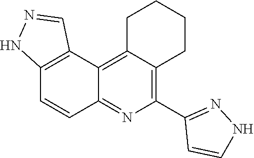 [SMILES: c1cc(-c2nc3ccc4[nH]ncc4c3c3c2CCCC3)n[nH]1]

Off-white solid (135 mg, 47%).

¹H NMR (500 MHz, DMSO-d₆) δ 8.61 (s, 1H), 8.03-7.94 (m, 2H), 7.91-7.77 (m, 1H), 6.92 (s, 1H), 3.31 (t, J=6.5 Hz, 2H), 3.08 (t, J=6.2 Hz, 2H), 1.96 (q, J=6.2 Hz, 2H), 1.85-1.71 (m, 2H); ¹³C NMR (200 MHz, DMSO-d₆) δ 147.10, 145.88, 138.36, 137.58, 136.14, 135.75, 130.88, 130.30, 122.28, 121.82, 117.39, 115.57, 107.43, 30.32, 28.20, 21.97, 21.86; HRMS (ESI) m/z calcd for C₁₇H₁₆N₅ [M+H]⁺ 290.1405, found 290.1403.

### Example 21

### 7-(1-Methyl-1H-pyrazol-4-yl)-8,9,10,11-tetrahydro-3H-pyrazolo[4,3-a]phenanthridine (Compound 20)

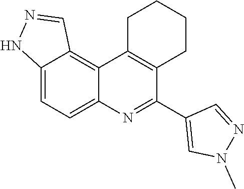 [SMILES: Cn1cc(-c2nc3ccc4[nH]ncc4c3c3c2CCCC3)cn1]

Off-white solid (64 mg, 21%).

¹H NMR (800 MHz, DMSO-d₆) δ 8.53 (s, 1H), 8.44 (d, J=5.3 Hz, 1H), 8.12 (d, J=5.4 Hz, 1H), 8.03 (t, J=7.3 Hz, 1H), 7.96 (t, J=7.2 Hz, 1H), 3.98 (s, 3H), 3.25-3.08 (m, 2H), 2.94-2.83 (m, 2H), 1.99-1.89 (m, 2H), 1.83-1.75 (m, 2H); ¹³C NMR (200 MHz, DMSO-d₆) δ 148.71, 145.80, 140.35, 138.14, 137.32, 135.56, 133.22, 129.96, 123.44, 121.49, 117.17, 116.65, 115.23, 39.25, 30.37, 28.12, 21.82, 21.74.

HRMS (ESI) m/z calcd for C₁₈H₁₈N₅ [M+H]⁺ 304.1562, found 304.1565.

### Example 22

### (4-methylpiperazin-1-yl) (4-(8,9,10,11-tetrahydro-3H-pyrazolo[4,3-a]phenanthridin-7-yl)phenyl)methanone (Compound 21)

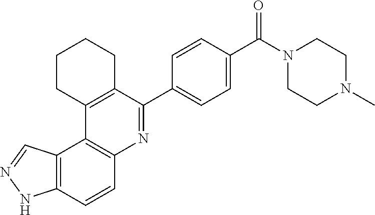 [SMILES: CN1CCN(C(=O)c2ccc(-c3nc4ccc5[nH]ncc5c4c4c3CCCC4)cc2)CC1]

Method C: Off white solid (510 mg, 4 mmol, 30%).

¹H NMR (500 MHz, (CD₃)₂SO) δ 8.57 (s, 1H), 7.86 (d, J=9.0 Hz, 1H), 7.82 (s, 1H), 7.61 (d, J=7.9 Hz, 2H), 7.47 (d, J=8.0 Hz, 2H), 3.53 (s, 4H), 3.34 (t, J=6.8 Hz, 2H), 2.79 (t, J=6.3 Hz, 2H), 2.39-2.32 (m, 4H), 2.22 (s, 3H), 2.07-1.98 (m, 2H), 1.77 (m, 2H); ¹³C NMR (126 MHz, (CD₃)₂SO) δ 169.4, 156.1, 143.8, 142.5, 142.4, 135.8, 129.6, 129.5, 129.2, 128.1, 127.0, 122.1, 121.0, 116.3, 114.5, 55.0, 46.0, 29.7, 28.8, 22.6, 22.5.

HRMS (ESI) m/z calcd for C₂₆H₂₈N₅O [M+H]⁺ 426.2289, found 426.2290.

### Example 23

### 1-(4-(4-(8,9,10,11-tetrahydro-3H-pyrazolo[4,3-a]phenanthridin-7-yl)benzoyl)piperazin-1-yl)ethan-1-one (Compound 22)

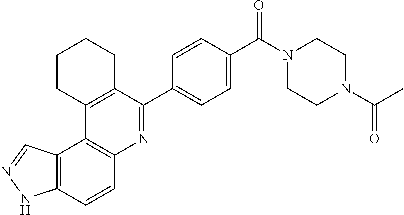 [SMILES: CC(=O)N1CCN(C(=O)c2ccc(-c3nc4ccc5[nH]ncc5c4c4c3CCCC4)cc2)CC1]

Method C: Off white solid (13.8 mg, 0.5 mmol, 5.7%).

¹H NMR (500 MHz, (CD₃)₂SO) δ 8.56 (s, 1H), 7.86 (d, J=9.1 Hz, 1H), 7.81 (d, J=9.8 Hz, 1H), 7.65-7.61 (m, 2H), 7.54-7.49 (m, 2H), 3.57-3.50 (m, 6H), 3.34 (q, J=9.8, 7.4 Hz, 4H), 2.79 (t, J=6.1 Hz, 2H), 2.05-1.97 (m, 5H), 1.79-1.73 (m, 2H); ¹³C NMR (126 MHz, (CD₃)₂SO) δ 169.7, 169.1, 156.1, 143.8, 142.6, 142.6, 135.5, 129.6, 129.6, 129.3, 127.2, 122.1, 118.0, 116.5, 116.4, 114.5, 46.1, 41.4, 29.6, 28.8, 22.6, 22.5, 21.6.

HRMS (ESI) m/z calcd for C₂₇H₂₈N₅O₂ [M+H]⁺ 454.2237, found 454.2241.

### Example 24

### 4-(3,8,9,10,11,12-hexahydrocyclohepta[c]pyrazolo[4,3-f]quinolin-7-yl)phenyl) (4-methylpiperazin-1-yl)methanone (Compound 23)

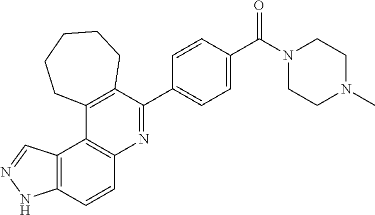 [SMILES: CN1CCN(C(=O)c2ccc(-c3nc4ccc5[nH]ncc5c4c4c3CCCCC4)cc2)CC1]

Method C: Off white solid (74 mg, 0.4 mmol, 42.1%).

¹H NMR (800 MHz, (CD₃)₂SO) δ 8.63 (s, 1H), 7.85 (d, J=9.0 Hz, 1H), 7.79 (s, 1H), 7.54 (d, J=8.0 Hz, 2H), 7.48 (d, J=8.1 Hz, 2H), 3.61 (s, 2H), 3.57-3.50 (m, 3H), 3.02-2.96 (m, 2H), 2.40-2.26 (m, 4H), 2.20 (s, 3H), 1.87 (m, 4H), 1.63 (m, 2H); ¹³C NMR (201 MHz, (CD₃)₂SO) δ 169.2, 161.1, 155.2, 148.7, 144.5, 142.7, 138.9, 135.5, 135.2, 129.6, 129.6, 127.0, 121.4, 116.1, 114.8, 55.1, 54.9, 54.3, 46.0, 45.0, 31.3, 31.0, 30.1, 27.3, 24.9.

HRMS (ESI) m/z calcd for C₂₇H₃₀N₅O [M+H]⁺ 440.2445, found 440.2444.

### Example 25

### (4-methylpiperazin-1-yl) (4-(3,8,9,10-tetrahydrocyclopenta[c]pyrazolo[4,3-f]quinolin-7-yl)phenyl)methanone (Compound 24)

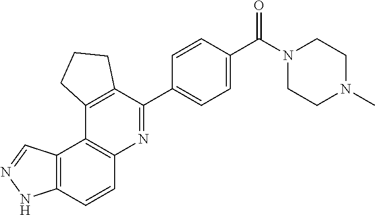 [SMILES: CN1CCN(C(=O)c2ccc(-c3nc4ccc5[nH]ncc5c4c4c3CCC4)cc2)CC1]

Method C: Off white solid (71 mg, 31%).

¹H NMR (800 MHz, (CD₃)₂SO) δ 8.51-8.44 (m, 1H), 7.96 (d, J=7.8 Hz, 2H), 7.94-7.84 (m, 2H), 7.53 (d, J=7.9 Hz, 2H), 3.65 (s, 2H), 3.48 (t, J=7.6 Hz, 4H), 3.28 (t, J=7.5 Hz, 2H), 2.54 (s, 1H), 2.41 (s, 4H), 2.28 (d, J=7.4 Hz, 2H), 2.26 (d, J=8.8 Hz, 3H); ¹³C NMR (201 MHz, (CD₃)₂SO) δ 169.2, 151.2, 149.7, 145.0, 141.5, 136.1, 135.9, 135.0, 129.2, 128.9, 127.6, 127.3, 119.4, 116.5, 115.0, 54.8, 47.5, 45.8, 41.9, 40.9, 33.5, 33.0, 24.9.

HRMS (ESI) m/z calcd for C₂₅H₂₆N₅O [M+H]⁺ 412.2132, found 412.2133.

### Example 26

### (4-methylpiperazin-1-yl) (4-(3,8,10,11-tetrahydropyrano[3,4-c]pyrazolo[4,3-f]quinolin-7-yl)phenyl)methanone (Compound 25)

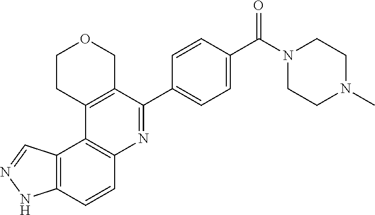 [SMILES: CN1CCN(C(=O)c2ccc(-c3nc4ccc5[nH]ncc5c4c4c3COCC4)cc2)CC1]

Method C: Off white solid (5 mg, 0.5 mmol, 4.7%).

¹H NMR (800 MHz, (CD₃)₂SO) δ 8.60 (s, 1H), 7.92 (d, J=9.0 Hz, 1H), 7.86 (d, J=9.3 Hz, 1H), 7.69-7.62 (m, 2H), 7.53-7.48 (m, 2H), 4.81 (s, 2H), 4.16 (t, J=5.9 Hz, 2H), 3.65 (s, 2H), 3.40 (t, J=6.1 Hz, 4H), 2.47-2.28 (m, 4H), 2.24 (s, 3H); ¹³C NMR (201 MHz, (CD₃)₂SO) δ 169.1, 152.9, 144.3, 140.8, 139.4, 138.8, 136.1, 135.7, 129.4, 129.2, 127.4, 127.2, 121.4, 116.0, 115.1, 66.8, 64.3, 54.8, 47.3, 45.8, 41.8, 40.5, 28.6.

HRMS (ESI) m/z calcd for C₂₅H₂₆N₅O₂ [M+H]⁺ 428.2081, found 428.2080.

### Example 27

### (4-(5-fluoro-8,9,10,11-tetrahydro-3H-pyrazolo[4,3-a]phenanthridin-7-yl)phenyl) (4-methylpiperazin-1-yl)methanone (Compound 26)

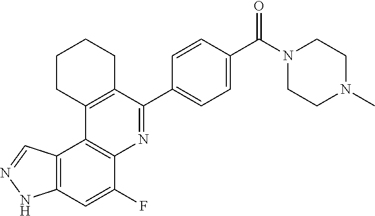 [SMILES: CN1CCN(C(=O)c2ccc(-c3nc4c(F)cc5[nH]ncc5c4c4c3CCCC4)cc2)CC1]

Method C: Off white solid (37 mg, 0.38 mmol, 22%).

¹H NMR (800 MHz, (CD₃)₂SO) δ 8.55 (s, 1H), 7.71 (d, J=10.0 Hz, 1H), 7.64 (d, J=7.6 Hz, 2H), 7.50 (d, J=7.0 Hz, 2H), 3.65 (s, 2H), 3.30 (s, 2H), 2.79 (d, J=6.7 Hz, 2H), 2.34 (d, J=68.6 Hz, 4H), 2.20 (d, J=6.3 Hz, 3H), 1.99 (q, J=6.6, 6.1 Hz, 2H), 1.74 (q, J=6.2 Hz, 2H); ¹³C NMR (201 MHz, (CD₃)₂SO) δ 169.2, 157.8 (1J=250 Hz), 156.1, 143.0, 141.9, 136.4, 135.8, 134.3, 130.7, 130.7, 129.5, 127.0, 122.7, 112.9, 98.4, 55.1, 54.6, 47.5, 46.0, 41.8, 29.6, 28.8, 22.3, 22.2.

HRMS (ESI) m/z calcd for C₂₆H₂₇FN₅O [M+H]⁺ 444.2194, found 444.2196.

### Example 28

### (2-fluoro-4-(8,9,10,11-tetrahydro-3H-pyrazolo[4,3-a]phenanthridin-7-yl)phenyl) (4-methylpiperazin-1-yl)methanone (Compound 27)

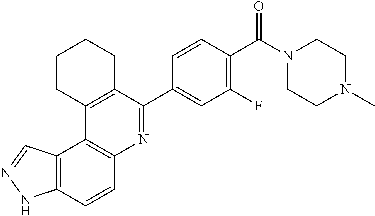 [SMILES: CN1CCN(C(=O)c2ccc(-c3nc4ccc5[nH]ncc5c4c4c3CCCC4)cc2F)CC1]

Method C: Yellow solid (18 mg, 0.25 mmol, 8%).

¹H NMR (500 MHz, (CD₃)₂SO) δ 8.57 (s, 1H), 7.86 (d, J=9.0 Hz, 1H), 7.81 (s, 1H), 7.47 (t, J=5.1 Hz, 3H), 3.68 (s, 2H), 2.81 (t, J=6.4 Hz, 2H), 2.42-2.27 (m, 5H), 2.22 (d, J=7.7 Hz, 4H), 2.01 (m, 2H), 1.82-1.71 (m, 2H); ¹³C NMR (126 MHz, (CD₃)₂SO) δ 164.4, 161.1, 158.7 (d, 1J=247 Hz), 154.8, 144.6 (d, ³J=7.56 Hz), 143.8, 142.7, 142.4, 129.6, 129.2, 128.8, 126.1, 123.9 (d, ²J=17.6 Hz), 122.3, 121.8, 116.7 (d, ²J=21 Hz), 116.2, 55.2, 46.1, 456.0, 29.7, 28.7, 22.5, 22.5. HRMS (ESI) m/z calcd for C₂₆H₂₇FN₅O [M+H]⁺ 444.2194, found 444.2194.

### Example 29

### (3-fluoro-4-(8,9,10,11-tetrahydro-3H-pyrazolo[4,3-a]phenanthridin-7-yl)phenyl) (4-methylpiperazin-1-yl)methanone (Compound 28)

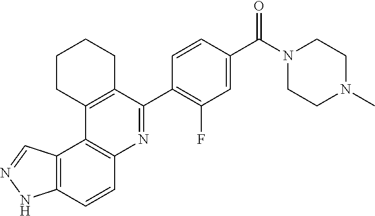 [SMILES: CN1CCN(C(=O)c2ccc(-c3nc4ccc5[nH]ncc5c4c4c3CCCC4)c(F)c2)CC1]

Method C: Off white solid (56 mg, 0.5 mmol, 25.3%).

¹H NMR (500 MHz, (CD₃)₂SO) δ 8.58 (s, 1H), 7.88 (d, J=9.1 Hz, 1H), 7.82 (s, 1H), 7.52 (t, J=7.6 Hz, 1H), 7.38-7.29 (m, 2H), 3.56 (s, 4H), 3.41-3.30 (m, 2H), 3.27-3.11 (m, 2H), 2.65 (t, J=6.2 Hz, 2H), 2.46 (s, 2H), 2.28 (s, 3H), 2.00 (p, J=6.5 Hz, 2H), 1.80 (tq, J=9.2, 6.2, 4.4 Hz, 2H); ¹³C NMR (126 MHz, (CD₃)₂SO) δ 167.9, 160.4 (d, 1J=245.7 Hz), 158.4, 151.6, 143.9, 142.4, 138.9, 138.3 (d, ³J=7.56 Hz), 136.3, 132.1, 130.1, 130.0, 129.4, 123.4, 122.4, 116.4, 114.6 (d, ²J=23 Hz), 54.6, 54.5, 45.6, 29.5, 27.4, 22.6, 22.2.

HRMS (ESI) m/z calcd for C₂₆H₂₇FN₅O [M+H]⁺ 444.2194, found 444.2193.

### Example 30

### (2-methoxy-4-(8,9,10,11-tetrahydro-3H-pyrazolo[4,3-a]phenanthridin-7-yl)phenyl) (4-methylpiperazin-1-yl)methanone (Compound 29)

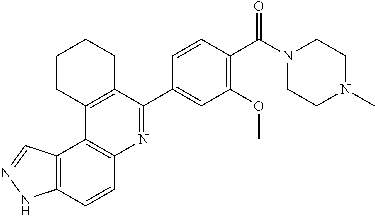 [SMILES: COc1cc(-c2nc3ccc4[nH]ncc4c3c3c2CCCC3)ccc1C(=O)N1CCN(C)CC1]

Method C: Off white solid (56 mg, 24%).

¹H NMR (800 MHz, (CD₃)₂SO) δ 8.57 (s, 1H), 7.87-7.82 (m, 1H), 7.82-7.74 (m, 1H), 7.28 (dd, J=7.4, 2.4 Hz, 1H), 7.12 (s, 1H), 7.05 (d, J=7.4 Hz, 1H), 3.74 (d, J=2.5 Hz, 3H), 3.65 (s, 2H), 3.38 (d, J=18.1 Hz, 1H), 3.30 (d, J=24.0 Hz, 2H), 2.67 (dd, J=16.0, 7.9 Hz, 1H), 2.52-2.44 (m, 2H), 2.43-2.27 (m, 4H), 2.22 (d, J=2.3 Hz, 3H), 2.04-1.92 (m, 2H), 1.81 (dd, J=14.1, 7.0 Hz, 1H), 1.75-1.68 (m, 1H); ¹³C NMR (201 MHz, (CD₃)₂SO) δ 169.1, 156.8, 154.8, 143.5, 141.4, 138.6, 137.5, 136.2, 131.5, 130.5, 130.4, 129.4, 121.9, 119.2, 116.5, 114.0, 110.2, 55.9, 46.0, 29.3, 27.0, 22.6, 22.1.

HRMS (ESI) m/z calcd for C₂₇H₂₉FN₅O₂ [M+H]⁺ 456.2394, found 456.2397.

### Example 31

### (2-methoxy-4-(8,9,10,11-tetrahydro-3H-pyrazolo[4,3-a]phenanthridin-7-yl)phenyl) (4-methylpiperazin-1-yl)methanone (Compound 30)

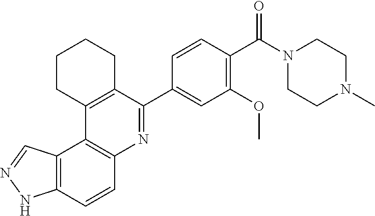 [SMILES: COc1cc(-c2nc3ccc4[nH]ncc4c3c3c2CCCC3)ccc1C(=O)N1CCN(C)CC1]

Method C: Off white solid (77 mg, 0.75 mmol, 22.6%).

¹H NMR (800 MHz, (CD₃)₂SO) δ 8.61-8.53 (m, 1H), 7.87 (dd, J=9.0, 4.2 Hz, 1H), 7.84 (s, 1H), 7.26 (dd, J=7.6, 4.2 Hz, 1H), 7.22 (d, J=4.1 Hz, 1H), 7.15 (dd, J=7.9, 3.8 Hz, 1H), 3.84 (d, J=4.4 Hz, 3H), 3.72-3.64 (m, 1H), 3.61 (d, J=16.7 Hz, 1H), 3.33-3.28 (m, 2H), 3.25-3.17 (m, 2H), 2.80 (q, J=6.1, 5.6 Hz, 2H), 2.34 (m, 3H), 2.26-2.22 (m, 1H), 2.21 (d, J=4.3 Hz, 3H), 2.01 (m, 2H), 1.76 (m, J=6.1 Hz, 2H); ¹³C NMR (201 MHz, (CD₃)₂SO) δ 166.7, 156.3, 155.0, 143.5, 143.4, 142.4, 138.7, 136.3, 129.5, 129.3, 127.7, 125.5, 122.0, 121.8, 116.5, 114.5, 112.6, 56.1, 55.1, 54.7, 46.6, 46.0, 41.3, 29.6, 28.7, 22.5, 22.4.

HRMS (ESI) m/z calcd for C₂₇H₃₀N₅O₂ [M+H]⁺ 456.2394, found 456.2394.

### Example 32

### (4-methylpiperazin-1-yl) (5-(8,9,10,11-tetrahydro-3H-pyrazolo[4,3-a]phenanthridin-7-yl)thiophen-2-yl)methanone (Compound 31)

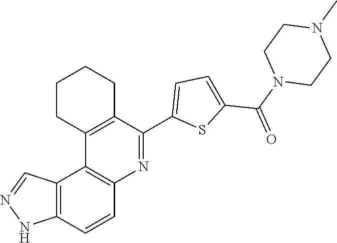 [SMILES: CN1CCN(C(=O)c2ccc(-c3nc4ccc5[nH]ncc5c4c4c3CCCC4)s2)CC1]

Method C: Off white solid (17 mg, 0.5 mmol, 7.9%).

¹H NMR (800 MHz, (CD₃)₂SO) δ 8.56 (s, 1H), 7.88 (d, J=9.0 Hz, 1H), 7.83-7.76 (m, 1H), 7.57 (d, J=3.9 Hz, 1H), 7.40 (d, J=3.8 Hz, 1H), 3.68 (t, J=5.2 Hz, 4H), 3.34 (s, 2H), 3.11 (t, J=6.2 Hz, 2H), 2.39 (t, J=5.2 Hz, 4H), 2.23 (s, 3H), 2.05-2.00 (m, 2H), 1.89-1.85 (m, 2H); ¹³C NMR (201 MHz, (CD₃)₂SO) δ 162.8, 148.3, 147.8, 143.3, 143.2, 138.8, 138.4, 136.4, 129.7, 129.2, 128.9, 127.5, 122.0, 116.2, 115.0, 55.0, 45.9, 29.9, 28.9, 22.4, 22.2.

HRMS (ESI) m/z calcd for C₂₄H₂₆N₅OS [M+H]⁺ 432.1852, found 432.1853.

### Example 33

### 1-(4-(4-(8,9,10,11-tetrahydro-3H-pyrazolo[4,3-a]phenanthridin-7-yl)benzoyl)piperazin-1-yl)ethan-1-one (Compound 32)

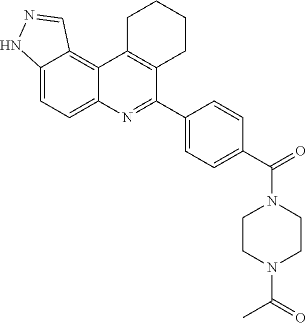 [SMILES: CC(=O)N1CCN(C(=O)c2ccc(-c3nc4ccc5[nH]ncc5c4c4c3CCCC4)cc2)CC1]

¹H NMR (500 MHz, DMSO) δ 8.56 (s, 1H), 7.84 (q, J=9.1 Hz, 2H), 7.67-7.60 (m, 2H), 7.56-7.48 (m, 2H), 3.51 (s, 8H), 2.79 (t, J=6.1 Hz, 2H), 2.48 (p, J=1.8 Hz, 1H), 2.02 (s, 2H), 2.02-1.97 (m, 3H), 1.74 (tt, J=8.7, 5.4 Hz, 2H); ¹³C NMR (126 MHz, DMSO) δ 169.6, 168.0, 156.2, 143.7, 142.6, 142.5, 138.7, 136.4, 135.5, 129.6, 129.4, 127.2, 122.0, 116.5, 114.6, 29.7, 28.9, 22.6, 22.5, 21.6.

HRMS (ESI), m/z calcd for C₂₈H₂₇N₄O₂ [M+H]⁺ 454.2243, found 454.2247.

### Example 34

### N-(1-(methylsulfonyl) piperidin-4-yl)-4-(8,9,10,11-tetrahydro-3H-pyrazolo[4,3-a]phenanthridin-7-yl)benzamide (Compound 33)

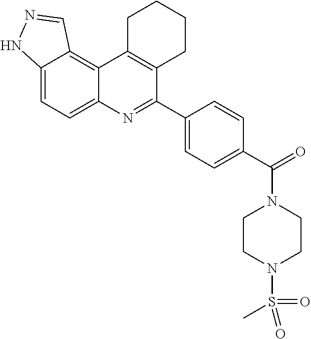 [SMILES: CS(=O)(=O)N1CCN(C(=O)c2ccc(-c3nc4ccc5[nH]ncc5c4c4c3CCCC4)cc2)CC1]

¹H NMR (500 MHz, DMSO): δ 8.58 (s, 1H), 7.85 (q, J=9.1 Hz, 2H), 7.68-7.61 (m, 2H), 7.55-7.45 (m, 2H), 3.64 (d, J=100.5 Hz, 4H), 3.36 (s, 2H), 3.19 (s, 4H), 2.91 (s, 3H), 2.80 (t, J=6.1 Hz, 2H), 2.06-1.97 (m, 2H), 1.77 (q, J=5.7 Hz, 2H); ¹³C NMR (126 MHz, DMSO) δ 169.5, 156.1, 143.6, 142.6, 138.8, 136.5, 135.3, 129.7, 129.4, 127.2, 122.0, 116.4, 114.6, 45.9, 34.6, 28.9, 22.6, 22.5.

HRMS (ESI), m/z calcd for C₂₆H₂₇N503S [M+H]⁺ 490.1913, found 490.1917.

### Example 35

### Morpholino (4-(8,9,10,11-tetrahydro-3H-pyrazolo[4,3-a]phenanthridin-7-yl)phenyl)methanone (Compound 34)

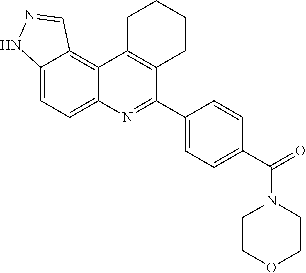 [SMILES: O=C(c1ccc(-c2nc3ccc4[nH]ncc4c3c3c2CCCC3)cc1)N1CCOCC1]

¹H NMR (500 MHz, DMSO) δ 8.57 (s, 1H), 7.85 (q, J=9.1 Hz, 2H), 7.66-7.58 (m, 2H), 7.53-7.46 (m, 2H), 3.63 (s, 8H), 3.39-3.33 (m, 2H), 2.79 (t, J=6.1 Hz, 2H), 2.05-1.96 (m, 2H), 1.76 (q, J=5.4 Hz, 2H); ¹³C NMR (126 MHz, DMSO): δ 169.4, 156.2, 143.7, 142.6, 142.5, 138.7, 136.4, 135.4, 129.6, 129.4, 127.2, 122.0, 116.5, 114.6, 66.6, 31.2, 29.7, 28.9, 22.6, 22.5.

HRMS (ESI), m/z calcd for C₂₅H₂₄N₄O₂ [M+H]⁺ 413.1978, found 413.1988.

### Example 36

### (4-hydroxy-4-methylpiperidin-1-yl) (4-(8,9,10,11-tetrahydro-3H-pyrazolo[4,3-a]phenanthridin-7-yl)phenyl)methanone (Compound 35)

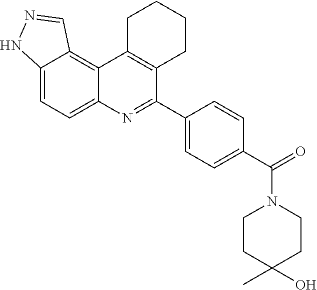 [SMILES: CC1(O)CCN(C(=O)c2ccc(-c3nc4ccc5[nH]ncc5c4c4c3CCCC4)cc2)CC1]

¹H NMR (500 MHz, DMSO) δ 8.57 (s, 1H), 7.85 (q, J=9.1 Hz, 2H), 7.68-7.54 (m, 2H), 7.47 (d, J=8.0 Hz, 2H), 4.44 (s, 1H), 3.36 (s, 4H), 3.24 (s, 2H), 2.80 (t, J=6.1 Hz, 2H), 2.01 (d, J=6.5 Hz, 2H), 1.76 (d, J=6.3 Hz, 2H), 1.46 (s, 4H), 1.16 (s, 3H); ¹³C NMR (126 MHz, DMSO) δ 169.1, 142.6, 142.1, 136.3, 129.5, 126.8, 114.6, 40.5, 40.4, 40.3, 30.3, 28.9, 22.6, 22.5.

HRMS (ESI), m/z calcd for C₂₇H₂₈N₄O₂ [M+H]⁺ 441.2291 found 441.2294.

### Example 37

### (1,1-dioxidothiomorpholino) (4-(8,9,10,11-tetrahydro-3H-pyrazolo[4,3-a]phenanthridin-7-yl)phenyl)methanone (Compound 36)

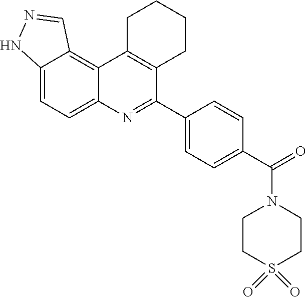 [SMILES: O=C(c1ccc(-c2nc3ccc4[nH]ncc4c3c3c2CCCC3)cc1)N1CCS(=O)(=O)CC1]

¹H NMR (500 MHz, DMSO) δ 8.58 (s, 1H), 7.85 (q, J=9.1 Hz, 2H), 7.65 (d, J=8.2 Hz, 2H), 7.60 (d, J=8.2 Hz, 2H), 4.10-3.71 (m, 4H), 3.36 (s, 2H), 3.29-3.12 (m, 4H), 2.80 (t, J=6.1 Hz, 2H), 2.01 (t, J=6.0 Hz, 2H), 1.76 (q, J=5.7 Hz, 2H); ¹³C NMR (126 MHz, DMSO) δ 169.9, 142.79, 142.6, 136.5, 134.9, 129.7, 129.4, 127, 122.0, 116.5, 114.6, 51.3, 29.7, 28.9, 22.6, 22.5. HRMS (ESI), m/z calcd for C₂₅H₂₄N403S [M+H]⁺ 461.1647 found 461.1651.

### Example 38

### ((2S,6R)-2,6-dimethylmorpholino) (4-(8,9,10,11-tetrahydro-3H-pyrazolo[4,3-a]phenanthridin-7-yl)phenyl)methanone (Compound 37)

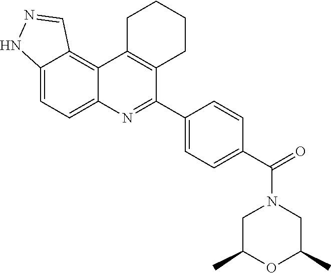 [SMILES: C[C@@H]1CN(C(=O)c2ccc(-c3nc4ccc5[nH]ncc5c4c4c3CCCC4)cc2)C[C@H](C)O1]

¹H NMR (500 MHz, DMSO) δ 8.57 (s, 1H), 7.85 (q, J=9.1 Hz, 2H), 7.66-7.57 (m, 2H), 7.52-7.44 (m, 2H), 3.57 (dd, J=12.7, 6.3 Hz, 3H), 3.36 (d, J=6.4 Hz, 2H), 3.30 (s, 2H), 2.80 (t, J=6.1 Hz, 4H), 2.05-1.95 (m, 2H), 1.76 (q, J=5.6 Hz, 2H), 1.08 (d, J=59.6 Hz, 6H); ¹³C NMR (126 MHz, DMSO) δ 169.1, 156.2, 143.7, 142.6, 142.4, 138.7, 136.4, 135.5, 129.6, 129.4, 127.3, 122.0, 116.5, 114.6, 71.7, 29.7, 28.9, 22.6, 22.5, 18.9.

HRMS (ESI), m/z calcd for C₂₇H₂₈N₄O₂ [M+H]⁺ 441.2290, found 441.22922.

### Example 39

### (4-(3,8,9,10,11,12-hexahydrocyclohepta[c]pyrazolo[4,3-f]quinolin-7-yl)phenyl) (4-(methylsulfonyl)piperazin-1-yl)methanone (Compound 38)

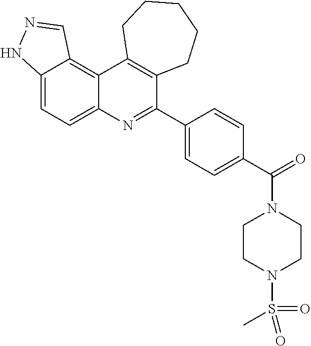 [SMILES: CS(=O)(=O)N1CCN(C(=O)c2ccc(-c3nc4ccc5[nH]ncc5c4c4c3CCCCC4)cc2)CC1]

¹H NMR (500 MHz, DMSO) δ 8.64 (s, 1H), 7.89-7.77 (m, 2H), 7.57 (d, J=8.2 Hz, 2H), 7.54 (d, J=8.2 Hz, 2H), 3.74 (s, 2H), 3.56 (s, 4H), 3.20 (s, 4H), 3.05-2.98 (m, 2H), 2.91 (s, 3H), 1.93-1.83 (m, 4H), 1.69-1.61 (m, 2H); ¹³C NMR (126 MHz, DMSO) δ 169.5, 155.2, 148.9, 144.5, 143.1, 139.0, 135.6, 135.5, 135.0, 129.7, 127.3, 121.4, 116.2, 115.0, 45.9, 34.6, 31.5, 31.1, 27.4, 25.0.

HRMS (ESI), m/z calcd for C₂₇H₂₉N503S [M+H]⁺ 504.2069, found 504.2070.

### Example 40

### N-(2-sulfamoylethyl)-4-(8,9,10,11-tetrahydro-3H-pyrazolo[4,3-a]phenanthridin-7-yl)benzamide (Compound 39)

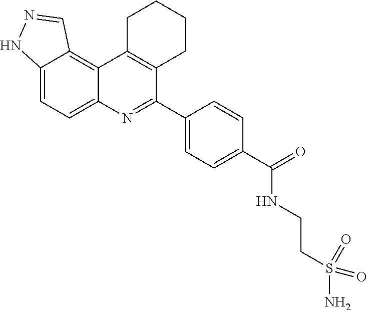 [SMILES: NS(=O)(=O)CCNC(=O)c1ccc(-c2nc3ccc4[nH]ncc4c3c3c2CCCC3)cc1]

¹H NMR (500 MHz, DMSO) δ 8.70 (t, J=5.7 Hz, 1H), 8.59 (s, 1H), 7.96-7.91 (m, 2H), 7.90-7.80 (m, 2H), 7.69-7.63 (m, 2H), 6.95 (s, 2H), 3.68 (dt, J=7.3, 5.8 Hz, 2H), 3.29-3.24 (m, 2H), 2.77 (t, J=6.1 Hz, 2H), 2.01 (td, J=7.2, 4.6 Hz, 2H), 1.75 (qd, J=6.0, 4.1 Hz, 2H); ¹³C NMR (126 MHz, DMSO) δ 166.6, 156.2, 144.2, 143.7, 142.6, 136.5, 133.8, 129.7, 129.6, 127.4, 122.1, 116.5, 114.6, 54.1, 35.3, 29.7, 28.9, 22.7, 22.5.

HRMS (ESI), m/z calcd for C₂₃H₂₃N503S [M+H]⁺ 450.1600, found 450.1602.

### Example 41

### N-(2-(N-methylsulfamoyl)ethyl)-4-(8,9,10,11-tetrahydro-3H-pyrazolo[4,3-a]phenanthridin-7-yl)benzamide (Compound 40)

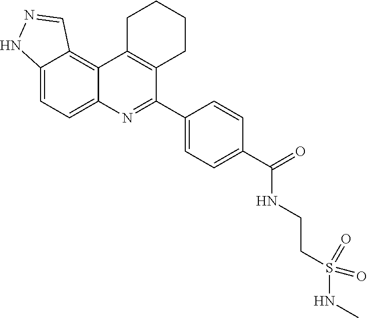 [SMILES: CNS(=O)(=O)CCNC(=O)c1ccc(-c2nc3ccc4[nH]ncc4c3c3c2CCCC3)cc1]

¹H NMR (500 MHz, DMSO) δ 8.71 (t, J=5.7 Hz, 1H), 8.57 (s, 1H), 7.94 (d, J=8.1 Hz, 2H), 7.85 (q, J=9.1 Hz, 2H), 7.66 (d, J=8.1 Hz, 2H), 7.03 (q, J=4.9 Hz, 1H), 3.63 (dt, J=7.9, 5.9 Hz, 2H), 3.36 (d, J=6.8 Hz, 2H), 3.30-3.27 (m, 2H), 2.77 (t, J=6.2 Hz, 2H), 2.61 (d, J=4.9 Hz, 3H), 2.04-1.98 (m, 2H), 1.75 (tt, J=8.5, 5.2 Hz, 2H); ¹³C NMR (126 MHz, DMSO) δ 166.6, 156.2, 144.2, 143.7, 142.6, 138.8, 136.5, 133.7, 129.7, 129.6, 129.4, 127.4, 122.0, 116.5, 114.6, 48.9, 34.9, 31.2, 29.7, 29.1, 28.9, 22.6, 22.5.

HRMS (ESI), m/z calcd for C₂₄H₂₅N503S [M+H]⁺ 464.1756, found 464.1759.

### Example 42

### N-methyl-N-(2-sulfamoylethyl)-4-(8,9,10,11-tetrahydro-3H-pyrazolo[4,3-a]phenanthridin-7-yl)benzamide (Compound 41)

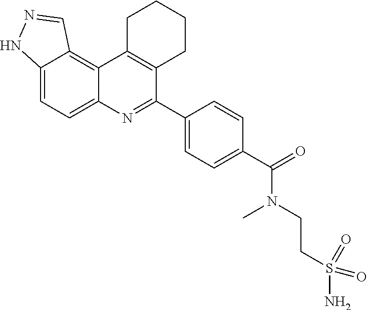 [SMILES: CN(CCS(N)(=O)=O)C(=O)c1ccc(-c2nc3ccc4[nH]ncc4c3c3c2CCCC3)cc1]

¹H NMR (500 MHz, DMSO) δ 8.57 (s, 1H), 7.85 (d, J=4.9 Hz, 2H), 7.62 (d, J=7.9 Hz, 2H), 7.51 (d, J=7.7 Hz, 2H), 6.94 (d, J=27.1 Hz, 2H), 3.75 (d, J=89.0 Hz, 2H), 3.36 (d, J=7.2 Hz, 4H), 3.01 (s, 3H), 2.78 (t, J=6.1 Hz, 2H), 2.00 (tt, J=8.6, 4.6 Hz, 2H), 1.78-1.72 (m, 2H); ¹³C NMR (126 MHz, DMSO) δ 170.7, 156.2, 143.6, 142.6, 142.4, 138.7, 136.4, 136.0, 129.7, 129.5, 127.1, 126.7, 122.0, 116.5, 114.5, 51.8, 43.1, 38.2, 28.9, 22.6, 22.5.

HRMS (ESI), m/z calcd for C₂₄H₂₅N503S [M+H]⁺ 464.1756, found 464.1761.

### Example 43

### 7-(1-ethyl-3-isopropyl-1H-pyrazol-4-yl)-8,9,10,11-tetrahydro-3H-pyrazolo[4,3-a]phenanthridine (Compound 42)

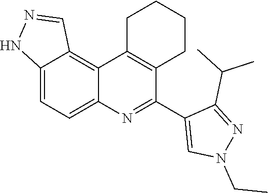 [SMILES: CCn1cc(-c2nc3ccc4[nH]ncc4c3c3c2CCCC3)c(C(C)C)n1]

¹H NMR (500 MHz, MeOD) δ 8.60 (s, 1H), 7.85 (t, J=7.6 Hz, 2H), 7.73 (s, 1H), 4.23 (q, J=7.2 Hz, 2H), 3.43 (d, J=6.7 Hz, 2H), 3.00 (h, J=6.9 Hz, 1H), 2.77 (t, J=6.2 Hz, 2H), 2.09 (td, J=7.3, 4.6 Hz, 2H), 1.95-1.83 (m, 2H), 1.50 (t, J=7.2 Hz, 3H), 1.16 (d, J=7.0 Hz, 7H); ¹³C NMR (126 MHz, MeOD) δ 156.3, 143.2, 135.9, 131.3, 129.4, 128.4, 117.8, 116.2, 113.7, 46.2, 29.6, 28.2, 26.7, 22.2, 22.0, 21.3, 14.9.

HRMS (ESI), m/z calcd for C₂₂H₂₅N₅ [M+H]⁺ 360.2188, found 360.2189.

### Example 44

### 7-(1-isopropyl-3-methyl-1H-pyrazol-4-yl)-8,9,10,11-tetrahydro-3H-pyrazolo[4,3-a]phenanthridine (Compound 43)

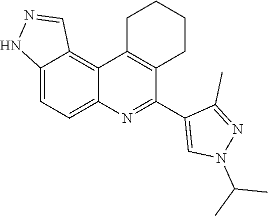 [SMILES: Cc1nn(C(C)C)cc1-c1nc2ccc3[nH]ncc3c2c2c1CCCC2]

¹H NMR (500 MHz, DMSO) δ 8.67-8.39 (m, 1H), 7.98 (s, 1H), 7.80 (q, J=9.1 Hz, 2H), 4.47 (hept, J=6.7 Hz, 1H), 2.84 (t, J=6.1 Hz, 2H), 2.26 (s, 3H), 1.98 (d, J=5.8 Hz, 2H), 1.80 (d, J=6.1 Hz, 2H), 1.44 (d, J=6.7 Hz, 6H); ¹³C NMR (126 MHz, DMSO) δ 146.7, 138.6, 136.3, 129.9, 128.6, 118.5, 114.1, 53.1, 40.5, 29.8, 28.5, 23.2, 22.6, 13.7.

HRMS (ESI), m/z calcd for C₂₁H₂₃N₅ [M+H]⁺ 346.2032, found 346.2034.

### Example 45

### (3-fluoro-4-(8,9,10,11-tetrahydro-3H-pyrazolo[4,3-a]phenanthridin-7-yl)phenyl) (4-(methylsulfonyl)piperazin-1-yl)methanone (Compound 44)

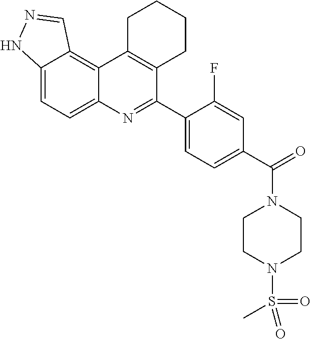 [SMILES: CS(=O)(=O)N1CCN(C(=O)c2ccc(-c3nc4ccc5[nH]ncc5c4c4c3CCCC4)c(F)c2)CC1]

¹H NMR (500 MHz, DMSO) δ 8.59 (s, 1H), 7.88 (d, J=9.1 Hz, 1H), 7.83 (d, J=9.0 Hz, 1H), 7.55 (t, J=7.5 Hz, 1H), 7.46-7.35 (m, 2H), 3.74 (s, 1H), 3.52 (s, 1H), 3.36 (t, J=6.6 Hz, 2H), 3.30 (s, 4H), 2.91 (s, 3H), 2.64 (s, 2H), 2.09-1.91 (m, 2H), 1.79 (s, 2H); ¹³C NMR (126 MHz, DMSO) δ 168.0, δ 159.35 (d, J=246.0 Hz), 151.7, 143.7, 142.5, 138.8, 138.0, 136.5, 132.2, 130.2, 130.1, 129.5, 123.6, 122.4, 116.4, 115.0, 114.8, 45.8, 34.6, 29.5, 27.4, 22.6, 22.1. HRMS (ESI), m/z calcd for C₂₆H₂₆FN₅O₃S [M+H]⁺ 508.1819, found 508.1817.

### Example 46

### 3-fluoro-N-(2-sulfamoylethyl)-4-(8,9,10,11-tetrahydro-3H-pyrazolo[4,3-a]phenanthridin-7-yl)benzamide (Compound 45)

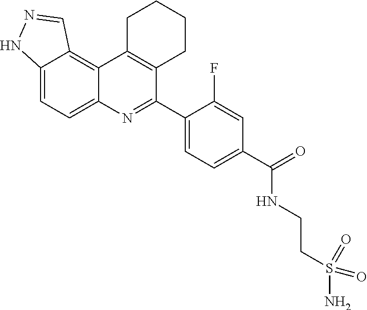 [SMILES: NS(=O)(=O)CCNC(=O)c1ccc(-c2nc3ccc4[nH]ncc4c3c3c2CCCC3)c(F)c1]

¹H NMR (500 MHz, DMSO) δ 8.81 (t, J=5.7 Hz, 1H), 8.59 (s, 1H), 7.88 (d, J=9.0 Hz, 1H), 7.84-7.74 (m, 3H), 7.58 (t, J=7.6 Hz, 1H), 6.96 (s, 2H), 3.68 (dt, J=8.4, 5.8 Hz, 2H), 3.30-3.26 (m, 2H), 2.64-2.57 (m, 2H), 1.98 (p, J=6.1 Hz, 2H), 1.81-1.74 (m, 2H), 1.25-1.18 (m, 2H); ¹³C NMR (126 MHz, DMSO) δ 165.3, δ 159.46 (d, J=244.7 Hz), 151.6, 142.5, 136.6, 136.6, 136.5, 132.0, 132.0, 131.8, 131.6, 130.1, 129.5, 123.8, 122.4, 116.4, 114.9, 114.7, 54.1, 53.9, 40.4, 35.3, 29.5, 27.4, 22.5, 22.1.

HRMS (ESI), m/z calcd for C₂₃H₂₂FN₅O₃S [M+H]⁺ 468.1506, found 468.1505.

### Example 47

### (2-fluoro-4-(8,9,10,11-tetrahydro-3H-pyrazolo[4,3-a]phenanthridin-7-yl)phenyl) (4-(methylsulfonyl)piperazin-1-yl)methanone (Compound 46)

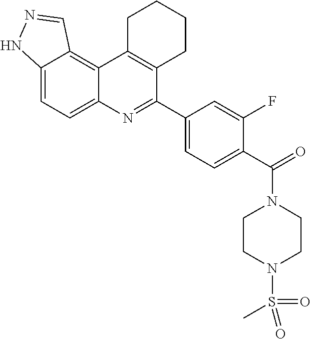 [SMILES: CS(=O)(=O)N1CCN(C(=O)c2ccc(-c3nc4ccc5[nH]ncc5c4c4c3CCCC4)cc2F)CC1]

¹H NMR (500 MHz, DMSO) δ 8.57 (s, 1H), 8.00-7.75 (m, 2H), 7.62-7.41 (m, 3H), 3.79 (t, J=5.0 Hz, 2H), 3.43 (t, J=5.0 Hz, 2H), 3.23 (t, J=5.2 Hz, 2H), 3.14 (t, J=5.0 Hz, 2H), 2.92 (s, 3H), 2.81 (t, J=6.1 Hz, 2H), 2.07 (s, 2H), 2.01 (qd, J=7.4, 4.4 Hz, 2H), 1.76 (h, J=5.5 Hz, 2H).

HRMS (ESI), m/z calcd for C₂₆H₂₆FN₅O₃S [M+H]⁺ 508.1819, found 508.1818.

### Example 48

### 2-fluoro-N-(2-sulfamoylethyl)-4-(8,9,10,11-tetrahydro-3H-pyrazolo[4,3-a]phenanthridin-7-yl)benzamide (Compound 47)

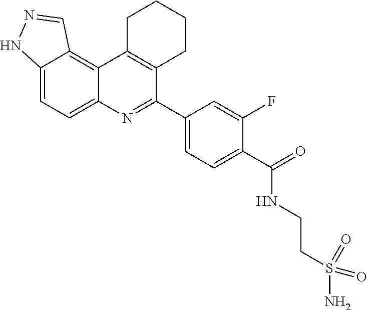 [SMILES: NS(=O)(=O)CCNC(=O)c1ccc(-c2nc3ccc4[nH]ncc4c3c3c2CCCC3)cc1F]

¹H NMR (500 MHz, DMSO) δ 8.58 (s, 1H), 8.50 (td, J=5.7, 3.3 Hz, 1H), 7.92-7.82 (m, 2H), 7.78 (t, J=7.7 Hz, 1H), 7.55-7.47 (m, 2H), 6.97 (s, 2H), 3.69 (dt, J=7.9, 6.0 Hz, 2H), 3.27 (dd, J=8.2, 6.2 Hz, 2H), 2.79 (t, J=6.2 Hz, 2H), 2.00 (dq, J=8.7, 4.5 Hz, 2H), 1.76 (ddt, J=11.4, 8.3, 4.9 Hz, 2H) ¹³C NMR (126 MHz, DMSO) δ 164.5, δ 157.69 (d, J=246.0 Hz), 154.8, 144.8, 144.8, 142.8, 138.8, 136.5, 129.6, 129.1, 129.0, 126.2, 123.4, 123.3, 117.1, 116.9, δ 47.38 45.12 (m), 41.4, 34.7, 31.2, 28.7, 22.5, 22.4.

HRMS (ESI), m/z calcd for C₂₃H₂₂FN₅O₃S [M+H]⁺ 468.1506, found 468.1504.

### Example 49

### 4-(4-(8,9,10,11-tetrahydro-3H-pyrazolo[4,3-a]phenanthridin-7-yl)benzoyl)piperazine-1-carboxamide (Compound 48)

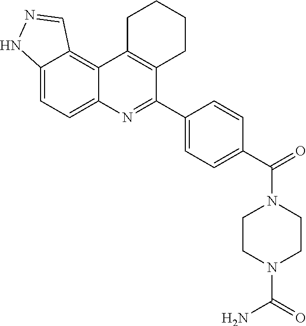 [SMILES: NC(=O)N1CCN(C(=O)c2ccc(-c3nc4ccc5[nH]ncc5c4c4c3CCCC4)cc2)CC1]

¹H NMR (500 MHz, DMSO) δ 8.57 (s, 1H), 7.85 (q, J=9.2 Hz, 2H), 7.63 (d, J=8.0 Hz, 2H), 7.51 (d, J=8.0 Hz, 2H), 6.07 (s, 2H), 3.60 (s, 2H), 3.31 (s, 8H), 2.79 (t, J=6.1 Hz, 2H), 2.00 (dq, J=8.8, 4.5 Hz, 2H), 1.79-1.71 (m, 2H); ¹³C NMR (126 MHz, DMSO) δ 169.5, 158.5, 156.1, 143.6, 142.6, 142.4, 138.7, 136.4, 135.6, 129.6, 129.4, 127.2, 122.0, 116.4, 114.6, 47.6, 43.9, 31.2, 28.9, 22.6, 22.5.

HRMS (ESI), m/z calcd for C₂₆H₂₆N₆O₂ [M+H]⁺ 455.2195, found 455.2197.

### Example 50

### (4-(methylsulfonyl)piperazin-1-yl) (5-(8,9,10,11-tetrahydro-3H-pyrazolo[4,3-a]phenanthridin-7-yl)pyridin-2-yl)methanone (Compound 49)

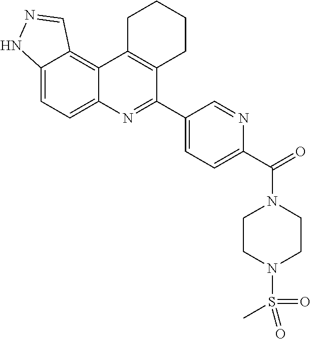 [SMILES: CS(=O)(=O)N1CCN(C(=O)c2ccc(-c3nc4ccc5[nH]ncc5c4c4c3CCCC4)cn2)CC1]

¹H NMR (500 MHz, DMSO) δ 8.80 (dd, J=2.2, 0.9 Hz, 1H), 8.58 (s, 1H), 8.18 (dd, J=8.0, 2.2 Hz, 1H), 7.87 (q, J=9.1 Hz, 2H), 7.74 (dd, J=7.9, 0.8 Hz, 1H), 3.82-3.77 (m, 2H), 3.64 (t, J=5.0 Hz, 2H), 3.40-3.32 (m, 3H), 3.25 (t, J=5.2 Hz, 2H), 3.17 (t, J=5.0 Hz, 2H), 2.92 (s, 3H), 2.89-2.81 (m, 3H), 2.08-1.98 (m, 2H), 1.77 (tt, J=8.8, 5.4 Hz, 2H); ¹³C NMR (126 MHz, DMSO) δ 167.2, 153.3, 153.0, 148.9, 143.8, 142.9, 138.8, 138.3, 137.6, 136.5, 129.8, 129.6, 123.3, 122.3, 116.4, 114.8, 46.7, 46.2, 45.7, 34.6, 29.7, 28.7, 22.5, 22.4.

HRMS (ESI), m/z calcd for C₂₅H₂₆N603S [M+H]⁺ 491.1865, found 491.1863.

### Example 51

### N-(2-sulfamoylethyl)-5-(8,9,10,11-tetrahydro-3H-pyrazolo[4,3-a]phenanthridin-7-yl)picolinamide (Compound 50)

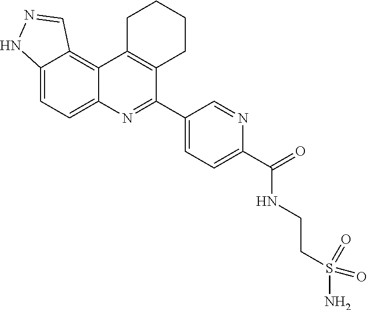 [SMILES: NS(=O)(=O)CCNC(=O)c1ccc(-c2nc3ccc4[nH]ncc4c3c3c2CCCC3)cn1]

¹H NMR (500 MHz, DMSO) δ 9.10 (t, J=6.0 Hz, 1H), 8.85 (d, J=2.0 Hz, 1H), 8.22 (dd, J=8.0, 2.2 Hz, 1H), 8.15 (d, J=8.0 Hz, 1H), 7.86 (q, J=9.2 Hz, 2H), 6.99 (s, 2H), 3.76 (q, J=6.7 Hz, 2H), 3.29 (t, J=7.0 Hz, 4H), 2.80 (t, J=6.1 Hz, 2H), 2.05-1.96 (m, 2H), 1.81-1.72 (m, 2H); ¹³C NMR (126 MHz, DMSO) δ 164.3, 153.1, 149.2, 149.1, 143.9, 143.0, 139.4, 138.7, 129.7, 129.6, 122.4, 121.8, 116.3, 114.9, 54.1, 34.8, 29.7, 28.6, 22.5, 22.4.

HRMS (ESI), m/z calcd for C₂₂H₂₂N603S [M+H]⁺ 451.1552, found 451.1551.

### Example 52

### (4-(methylsulfonyl)piperazin-1-yl) (6-(8,9,10,11-tetrahydro-3H-pyrazolo[4,3-a]phenanthridin-7-yl)pyridin-3-yl)methanone (Compound 51)

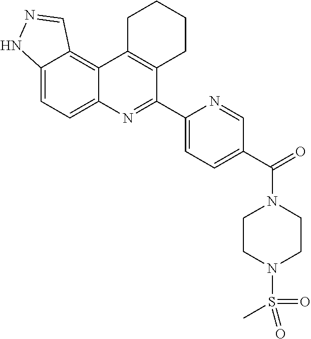 [SMILES: CS(=O)(=O)N1CCN(C(=O)c2ccc(-c3nc4ccc5[nH]ncc5c4c4c3CCCC4)nc2)CC1]

¹H NMR (500 MHz, DMSO) δ 8.72 (d, J=2.2 Hz, 1H), 8.57 (s, 1H), 8.02 (dd, J=8.0, 2.2 Hz, 1H), 7.92 (d, J=8.0 Hz, 1H), 7.86 (q, J=9.2 Hz, 2H), 3.77 (s, 2H), 3.62-3.47 (m, 2H), 3.34 (d, J=6.5 Hz, 3H), 2.99 (t, J=6.2 Hz, 3H), 2.92 (s, 3H), 2.08-1.91 (m, 4H), 1.79-1.71 (m, 2H); ¹³C NMR (126 MHz, DMSO) δ 167.5, 160.4, 153.9, 146.8, 143.3, 142.9, 138.9, 136.5, 136.1, 130.6, 130.1, 129.6, 124.6, 122.5, 116.4, 114.7, 45.7, 34.7, 31.1, 29.8, 28.2, 22.5, 22.3. HRMS (ESI), m/z calcd for C₂₅H₂₆N₆O₃S [M+H]⁺ 491.1865, found 491.1863.

### Example 53

### N-(2-sulfamoylethyl)-6-(8,9,10,11-tetrahydro-3H-pyrazolo[4,3-a]phenanthridin-7-yl)nicotinamide (Compound 52)

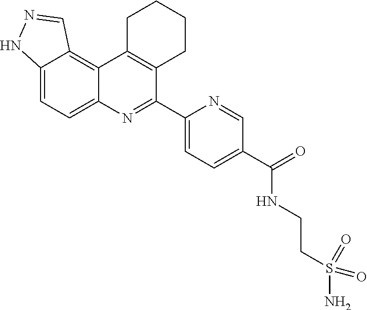 [SMILES: NS(=O)(=O)CCNC(=O)c1ccc(-c2nc3ccc4[nH]ncc4c3c3c2CCCC3)nc1]

¹H NMR (500 MHz, DMSO) δ 9.07 (dd, J=2.3, 0.9 Hz, 1H), 8.91 (t, J=5.7 Hz, 1H), 8.58 (s, 1H), 8.33 (dd, J=8.2, 2.3 Hz, 1H), 7.93 (dd, J=8.1, 0.8 Hz, 1H), 7.90-7.82 (m, 2H), 6.97 (s, 2H), 3.75-3.67 (m, 2H), 3.33-3.26 (m, 4H), 2.95 (t, J=6.2 Hz, 2H), 2.06 (s, 1H), 1.98 (qt, J=6.5, 3.3 Hz, 2H), 1.79-1.71 (m, 2H); ¹³C NMR (126 MHz, DMSO) δ 165.4, 161.7, 153.9, 147.4, 143.0, 136.6, 136.1, 130.1, 129.6, 128.9, 124.6, 122.6, 116.4, 114.7, 54.0, 35.3, 31.2, 28.2, 22.5, 22.3.

HRMS (ESI), m/z calcd for C₂₂H₂₂N603S [M+H]⁺ 451.1552, found 451.1553.

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. The terms “including” and “having” are defined as comprising (i.e., open language).

It will be appreciated by persons skilled in the art that the present disclosure is not limited by what has been particularly shown and described herein above. Rather the scope of the present disclosure includes both combinations and sub-combinations of the various features described hereinabove as well as variations and modifications which would occur to persons skilled in the art upon reading the specification and which are not in the prior art.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

1. A method for treating or inhibiting a disease associated with overacting of Stimulator of Interferon Genes (STING) comprising administering to a patient a therapeutically effective amount of a compound of formula (I): 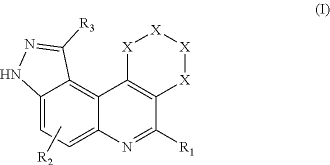
wherein
R₁ is selected from H, alkyl, cycloalkyl, heteroalkyl, cycloheteroalkyl, aryl, heteroaryl, alkenyl, alkynyl and amide, wherein the amide is selected from sulfonamide, benzamide, nicotinamide, picolinamide, pyrimidine carboxamide, imidazole carboxamide, pyrazole carboxamide and a derivative of any of the forgoing;
R₂ is selected from H, alkyl, heteroalkyl, aryl, heteroaryl, alkenyl, alkynyl, and halogen;
R₃ is selected from H, alkyl, heteroalkyl, aryl, heteroaryl, alkenyl, and alkynyl; and
each X is independently selected from O, S, SO, SO₂, CRR′, CNRR′, COR′, NR′ and Xₙ, wherein n is 0-2; wherein each R and R′ is independently selected from H, halogen, CF₃, CN, alkyl, aryl, heteroalkyl, heteroaryl, CONR₂R₂, COR₂, SO₂R₂, (C═O)NR₂, (C═NH)R₂, (C═O)OR₂, C₆H₅R₂, morpholine and piperazine, wherein R₂ is as defined above; or
R and R′, together with the atom to which they are attached, form a 4- to 6-membered cyclic or heterocyclic ring;
or a pharmaceutically acceptable salt, hydrate, tautomer, or optical isomer thereof or a pharmaceutical composition comprising the compound and a pharmaceutically acceptable carrier, excipient, or diluent.

2. The method of claim 1, wherein the disease associated with overacting of STING is selected from inflammatory diseases, autoimmune diseases, diabetes, cancer, traumatic brain injury, and fibrosis.

3. The method of claim 2, wherein the disease associated with overacting of STING is the inflammatory disease.

4. A compound of formula (IA): 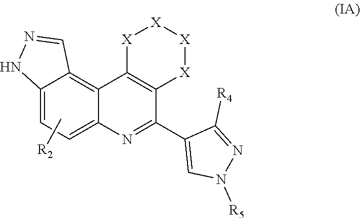
wherein
R₂ is selected from H, alkyl, heteroalkyl, aryl, heteroaryl, alkenyl, alkynyl, and halogen;
R₄ is selected from H, alkyl, cycloalkyl, heteroalkyl, heterocycloalkyl, aryl, heteroaryl, alkenyl, alkynyl, and amide, wherein the amide is selected from sulfonamide, benzamide, nicotinamide, picolinamide and a derivative of any of the forgoing; and R₄ is optionally substituted with OH, OR₂, halogen, CF₃, CN, NRR′, CONRR′, SO₂R₂, SO₂NRR′, NCOR₂ or NSO₂R₂, wherein each R and R′ is independently selected from H, halogen, CF₃, CN, alkyl, aryl, heteroalkyl, heteroaryl, CONR₂R₂, COR₂, SO₂R₂, (C═O)NR₂, (C═NH)R₂, (C═O)OR₂, C₆H₅R₂, morpholine and piperazine, wherein R₂ is as defined above; or
R and R′, together with the atom to which they are attached, form a 4- to 6-membered cyclic or heterocyclic ring;
R₅ is selected from H, alkyl, cycloalkyl, heteroalkyl, heterocycloalkyl, aryl, heteroaryl, alkenyl, alkynyl, and amide, wherein the amide is selected from sulfonamide, benzamide, nicotinamide, picolinamide and a derivative of any of the forgoing; and R₅ is optionally substituted with OH, OR₂, halogen, CF₃, CN, NRR′, CONRR′, SO₂R₂, SO₂NRR′, NCOR₂ or NSO₂R₂, wherein R₂, R and R′ are as defined above; and with the proviso that R₅ is not H, methyl, CF₃, CH₂—CH₂—OH, CH₂—CH₂—C₁, COOH, or optionally substituted phenyl, wherein the substitution on phenyl is OMe, COOMe, or amino;
R₄ and R₅ can be the same or different; and
each X is independently selected from O, S, SO, SO₂, CRR′, CNRR′, COR′, NR′ and Xₙ, wherein n is 0-2; wherein each R and R′ is independently selected from H, halogen, CF₃, CN, alkyl, aryl, heteroalkyl, heteroaryl, CONR₂R₂, COR₂, SO₂R₂, (C═O)NR₂, (C═NH)R₂, (C═O)OR₂, C₆H₅R₂, morpholine and piperazine, wherein R₂ is as defined above; or
R and R′, together with the atom to which they are attached, form a 4- to 6-membered cyclic or heterocyclic ring;
or a pharmaceutically acceptable salt, hydrate, tautomer, or optical isomer thereof.

5. The compound of claim 4, wherein the compound of formula (IA) is, 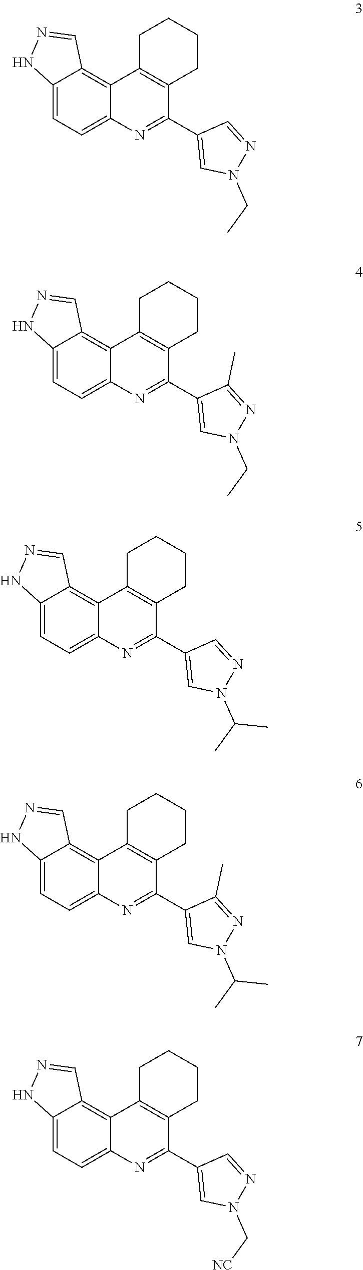 [SMILES: CCn1cc(-c2nc3ccc4[nH]ncc4c3c3c2CCCC3)cn1 ; CCn1cc(-c2nc3ccc4[nH]ncc4c3c3c2CCCC3)c(C)n1 ; CC(C)n1cc(-c2nc3ccc4[nH]ncc4c3c3c2CCCC3)cn1 ; Cc1nn(C(C)C)cc1-c1nc2ccc3[nH]ncc3c2c2c1CCCC2 ; N#CCn1cc(-c2nc3ccc4[nH]ncc4c3c3c2CCCC3)cn1] 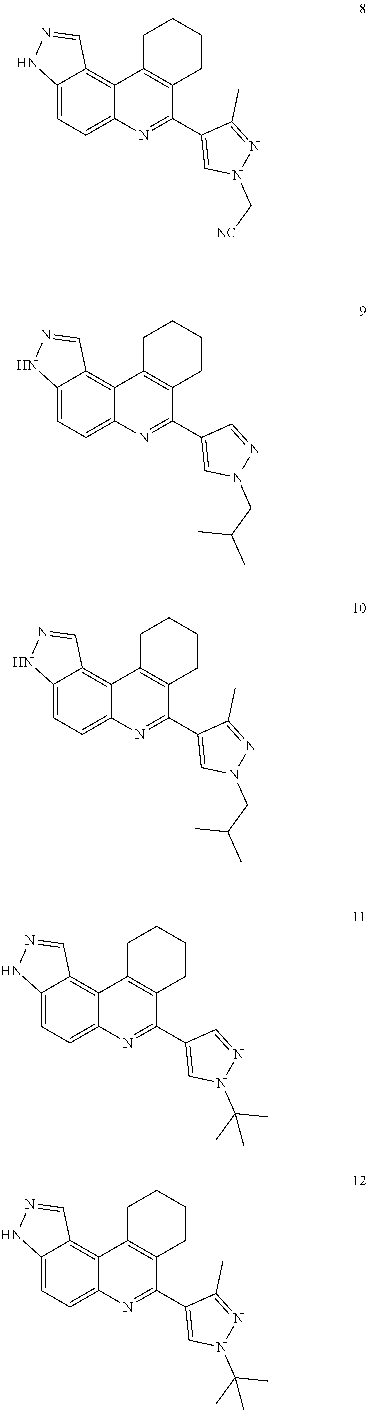 [SMILES: Cc1nn(CC#N)cc1-c1nc2ccc3[nH]ncc3c2c2c1CCCC2 ; CC(C)Cn1cc(-c2nc3ccc4[nH]ncc4c3c3c2CCCC3)cn1 ; Cc1nn(CC(C)C)cc1-c1nc2ccc3[nH]ncc3c2c2c1CCCC2 ; CC(C)(C)n1cc(-c2nc3ccc4[nH]ncc4c3c3c2CCCC3)cn1 ; Cc1nn(C(C)(C)C)cc1-c1nc2ccc3[nH]ncc3c2c2c1CCCC2] 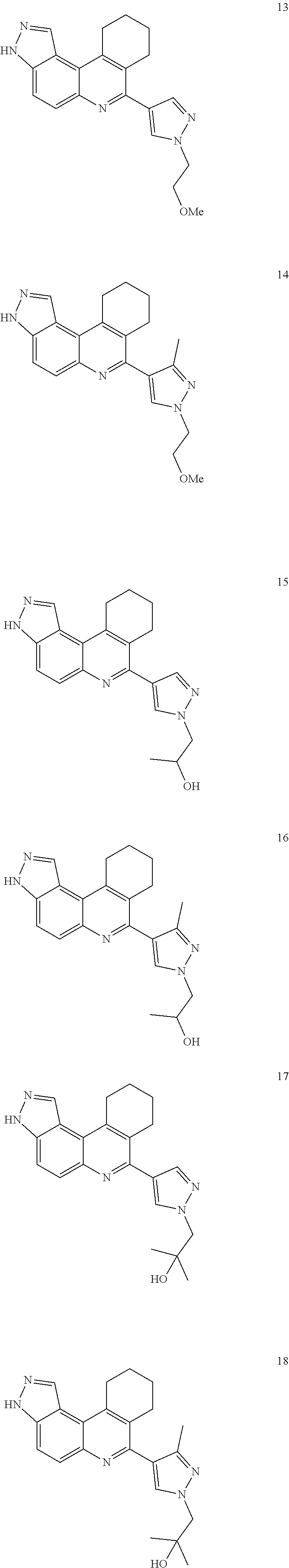 [SMILES: COCCn1cc(-c2nc3ccc4[nH]ncc4c3c3c2CCCC3)cn1 ; COCCn1cc(-c2nc3ccc4[nH]ncc4c3c3c2CCCC3)c(C)n1 ; CC(O)Cn1cc(-c2nc3ccc4[nH]ncc4c3c3c2CCCC3)cn1 ; Cc1nn(CC(C)O)cc1-c1nc2ccc3[nH]ncc3c2c2c1CCCC2 ; CC(C)(O)Cn1cc(-c2nc3ccc4[nH]ncc4c3c3c2CCCC3)cn1 ; Cc1nn(CC(C)(C)O)cc1-c1nc2ccc3[nH]ncc3c2c2c1CCCC2] 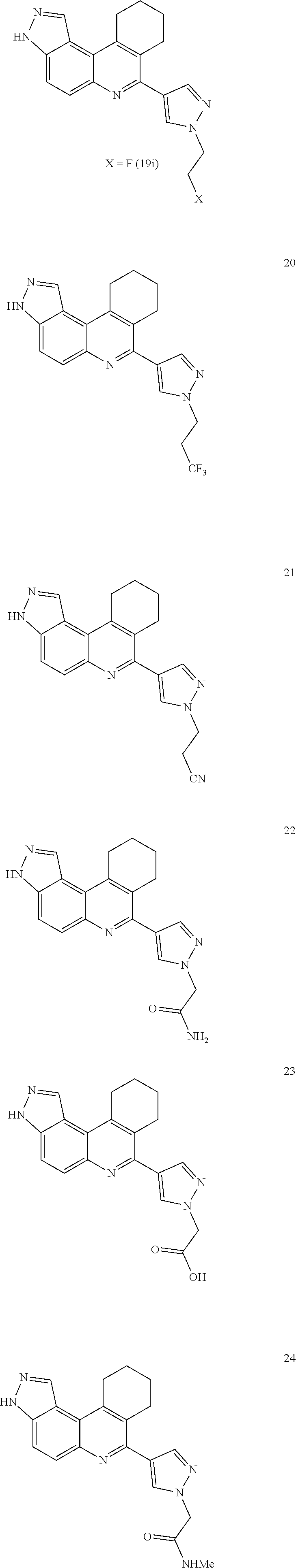 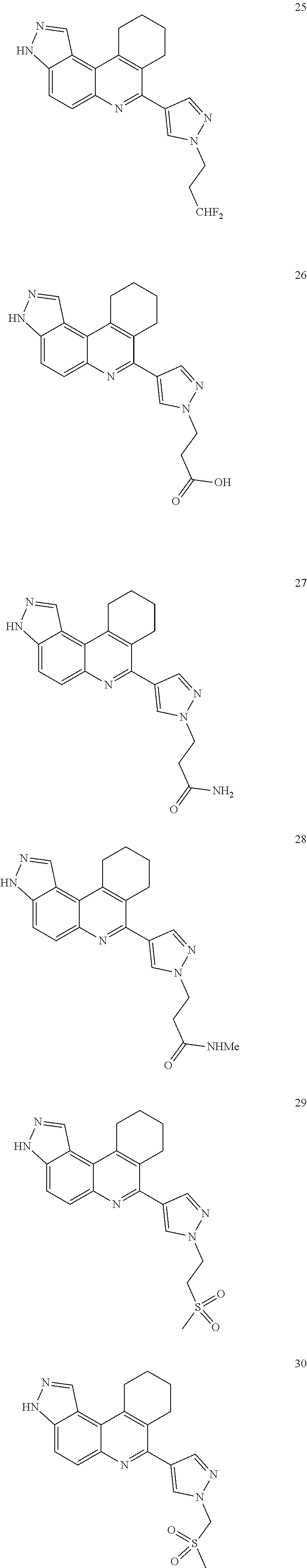 [SMILES: FC(F)CCn1cc(-c2nc3ccc4[nH]ncc4c3c3c2CCCC3)cn1 ; O=C(O)CCn1cc(-c2nc3ccc4[nH]ncc4c3c3c2CCCC3)cn1 ; NC(=O)CCn1cc(-c2nc3ccc4[nH]ncc4c3c3c2CCCC3)cn1 ; CNC(=O)CCn1cc(-c2nc3ccc4[nH]ncc4c3c3c2CCCC3)cn1 ; CS(=O)(=O)CCn1cc(-c2nc3ccc4[nH]ncc4c3c3c2CCCC3)cn1 ; CS(=O)(=O)Cn1cc(-c2nc3ccc4[nH]ncc4c3c3c2CCCC3)cn1] 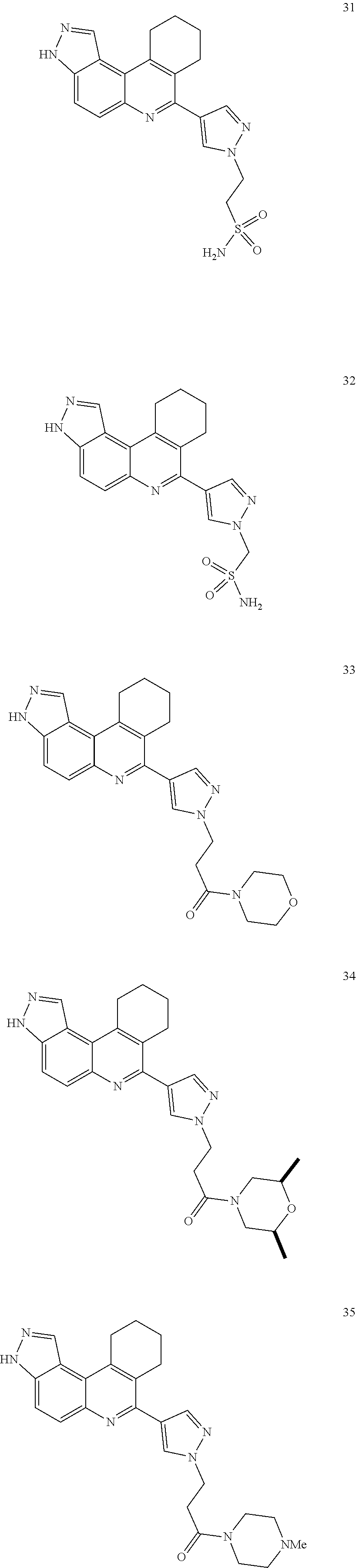 [SMILES: NS(=O)(=O)CCn1cc(-c2nc3ccc4[nH]ncc4c3c3c2CCCC3)cn1 ; NS(=O)(=O)Cn1cc(-c2nc3ccc4[nH]ncc4c3c3c2CCCC3)cn1 ; O=C(CCn1cc(-c2nc3ccc4[nH]ncc4c3c3c2CCCC3)cn1)N1CCOCC1 ; C[C@@H]1CN(C(=O)CCn2cc(-c3nc4ccc5[nH]ncc5c4c4c3CCCC4)cn2)C[C@H](C)O1 ; CN1CCN(C(=O)CCn2cc(-c3nc4ccc5[nH]ncc5c4c4c3CCCC4)cn2)CC1] 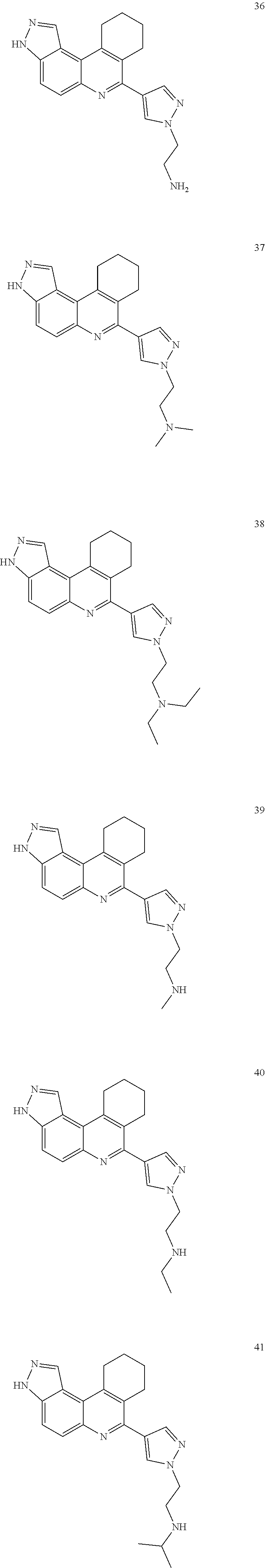 [SMILES: NCCn1cc(-c2nc3ccc4[nH]ncc4c3c3c2CCCC3)cn1 ; CN(C)CCn1cc(-c2nc3ccc4[nH]ncc4c3c3c2CCCC3)cn1 ; CCN(CC)CCn1cc(-c2nc3ccc4[nH]ncc4c3c3c2CCCC3)cn1 ; CNCCn1cc(-c2nc3ccc4[nH]ncc4c3c3c2CCCC3)cn1 ; CCNCCn1cc(-c2nc3ccc4[nH]ncc4c3c3c2CCCC3)cn1 ; CC(C)NCCn1cc(-c2nc3ccc4[nH]ncc4c3c3c2CCCC3)cn1] 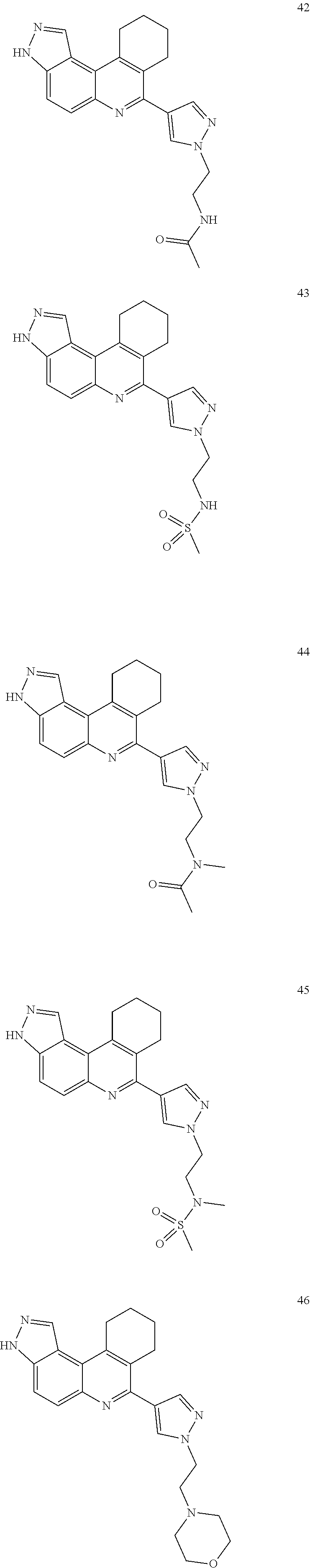 [SMILES: CC(=O)NCCn1cc(-c2nc3ccc4[nH]ncc4c3c3c2CCCC3)cn1 ; CS(=O)(=O)NCCn1cc(-c2nc3ccc4[nH]ncc4c3c3c2CCCC3)cn1 ; CC(=O)N(C)CCn1cc(-c2nc3ccc4[nH]ncc4c3c3c2CCCC3)cn1 ; CN(CCn1cc(-c2nc3ccc4[nH]ncc4c3c3c2CCCC3)cn1)S(C)(=O)=O ; c1nn(CCN2CCOCC2)cc1-c1nc2ccc3[nH]ncc3c2c2c1CCCC2] 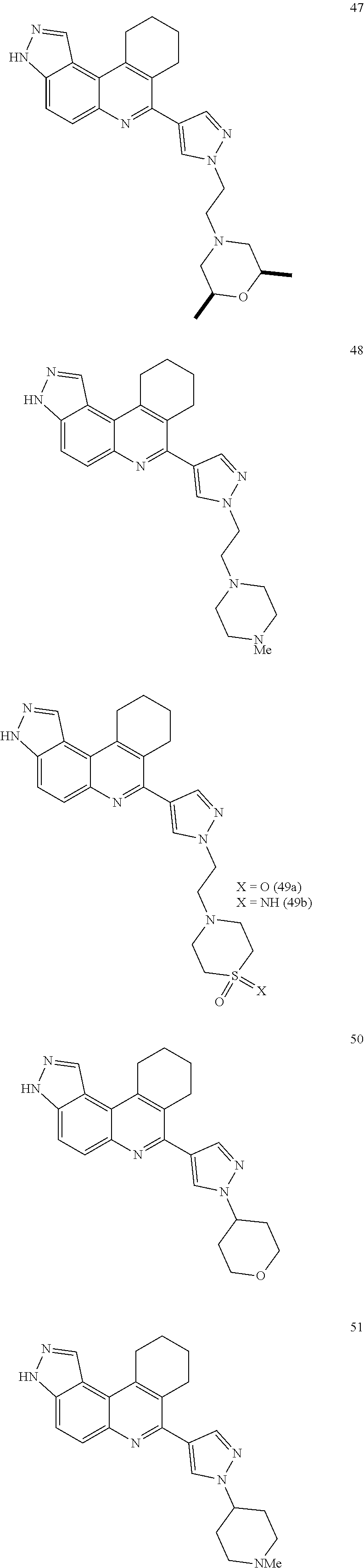 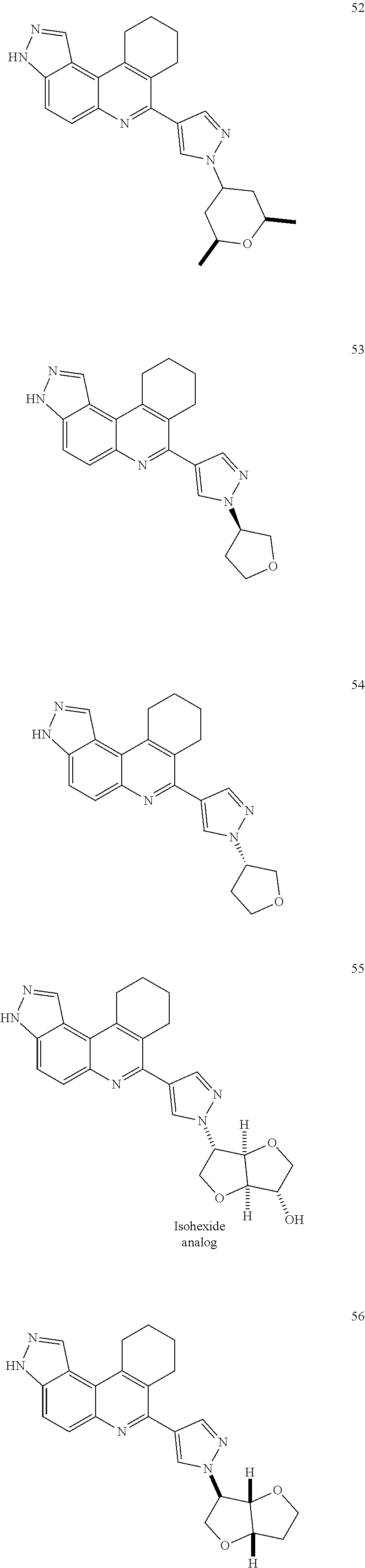 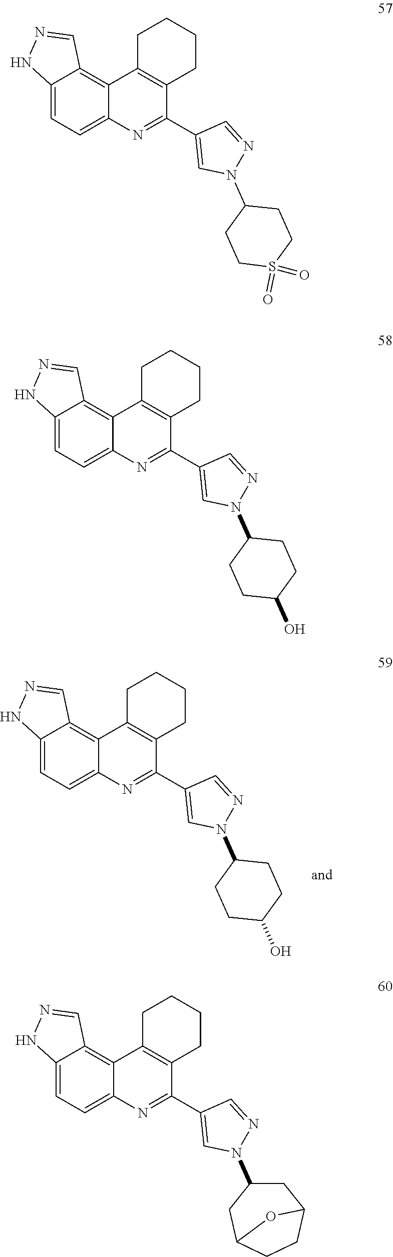
or a pharmaceutically acceptable salt, hydrate, tautomer, or optical isomer thereof.

6. The compound of claim 4, wherein the compound of formula (IA) is, 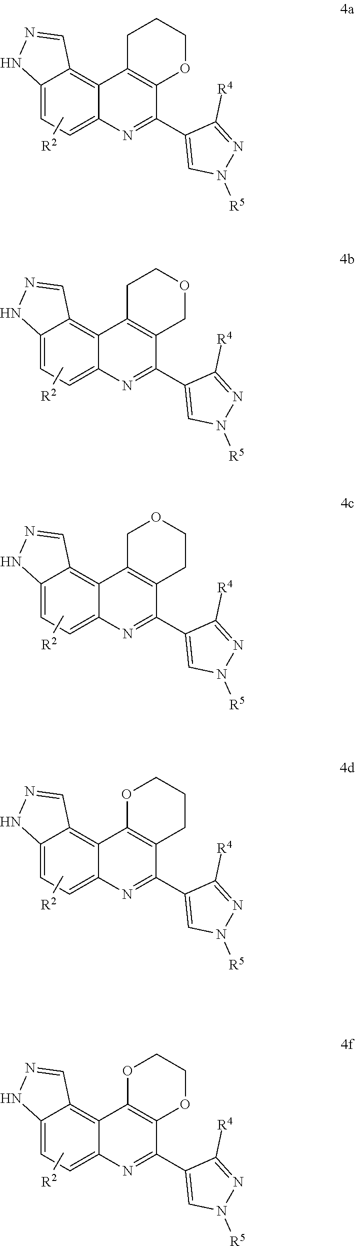 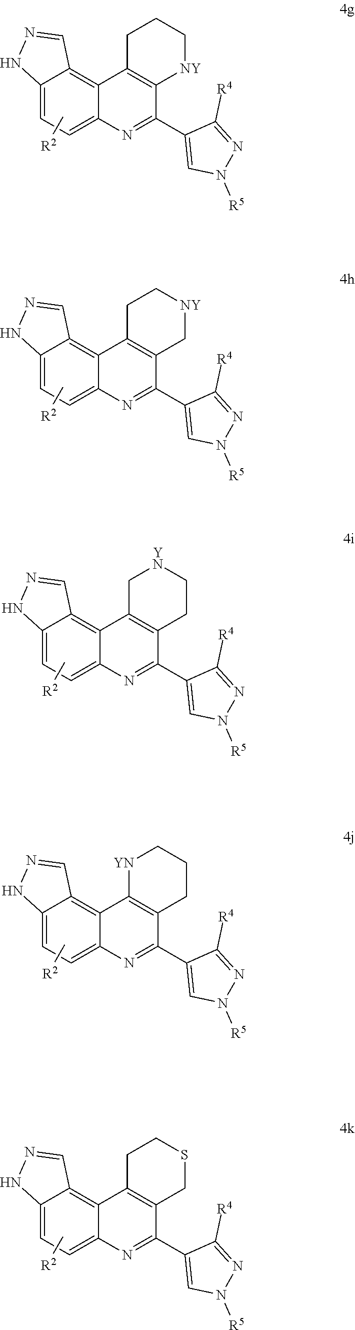 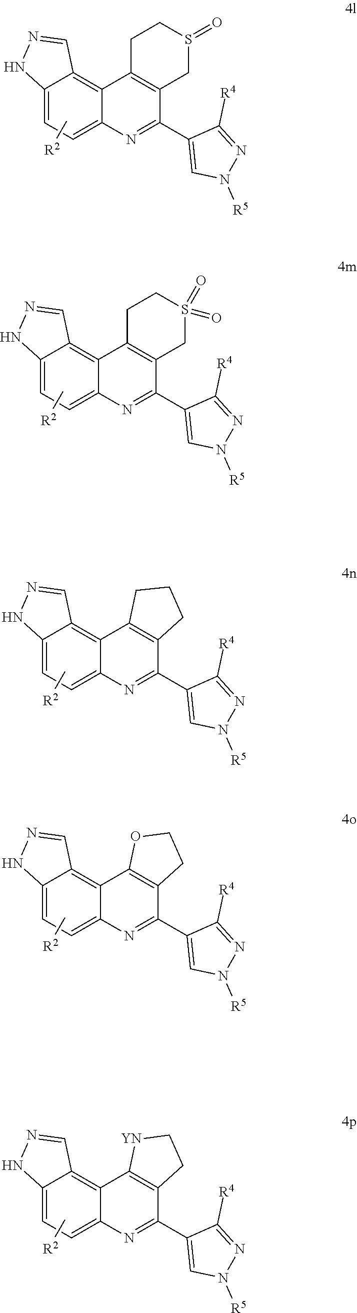 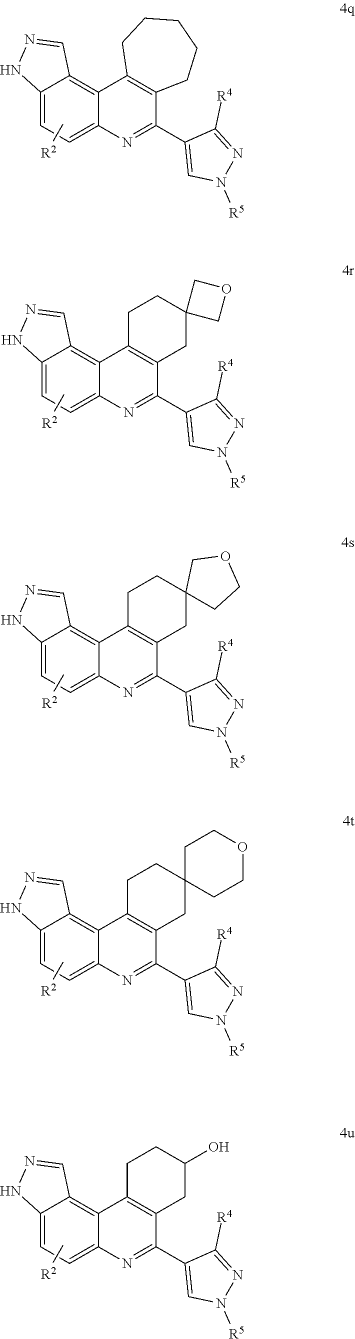 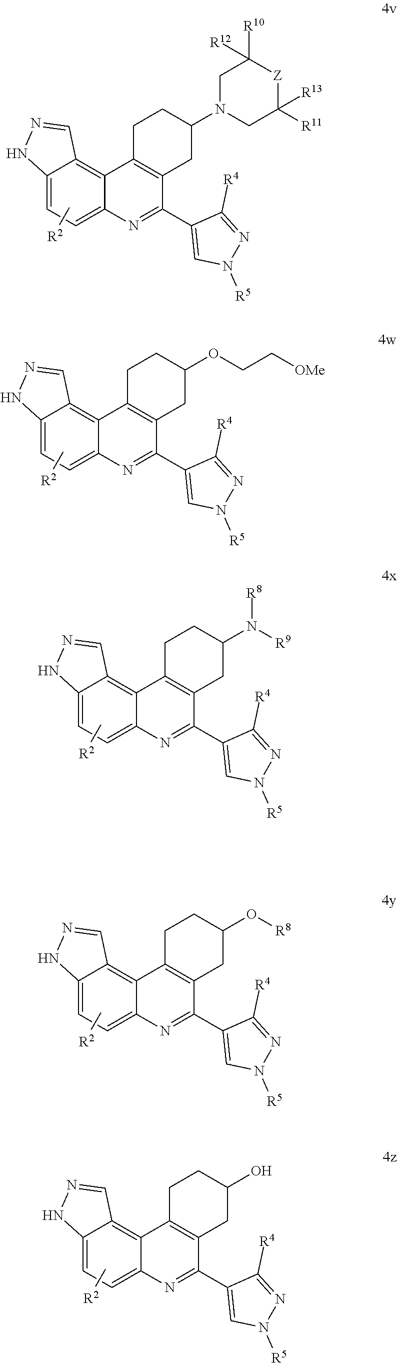 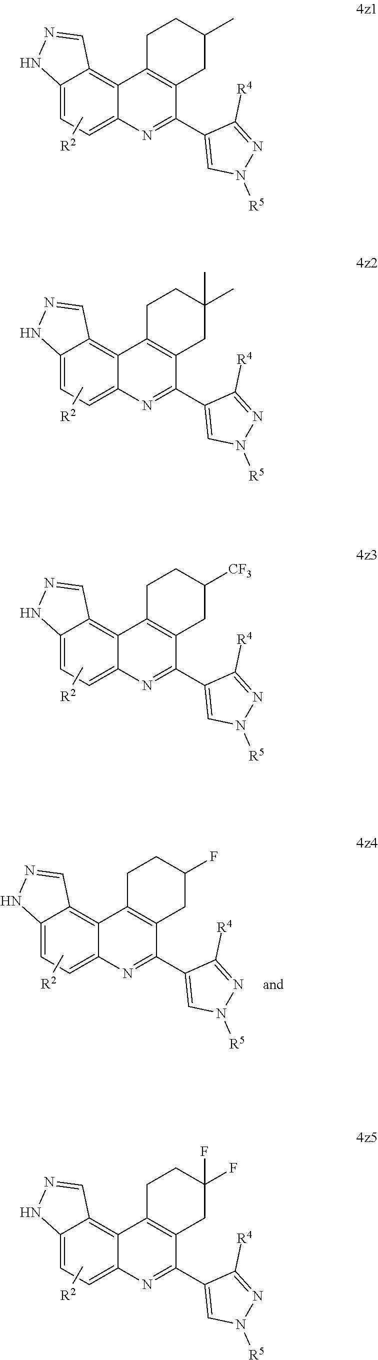
wherein
R₂ is selected from H, alkyl, heteroalkyl, aryl, heteroaryl, alkenyl, alkynyl, and halogen;
each R₄ and R₅ is independently selected from H, alkyl, cycloalkyl, heteroalkyl, heterocycloalkyl, aryl, heteroaryl, alkenyl, alkynyl, and amide, wherein the amide is selected from sulfonamide, benzamide, nicotinamide, picolinamide and a derivative of any of the forgoing, wherein alkyl is optionally substituted with OH, OR₂, halogen, CF₃, CN, NRR′, CONRR′, SO₂R₂, SO₂NRR′, NCOR₂ or NSO₂R₂, wherein each R and R′ is independently selected from H, halogen, CF₃, CN, alkyl, aryl, heteroalkyl, heteroaryl, CONR₂R₂, COR₂, SO₂R₂, (C═O)NR₂, (C═NH)R₂, (C═O)OR₂, C₆H₅R₂, morpholine and piperazine, wherein R₂ is as defined above; or
R and R′, together with the atom to which they are attached, form a 4- to 6-membered cyclic or heterocyclic ring;
each R₈ and R₉ is independently selected from H, methyl, alkyl, and heteroalkyl;
each R₁₀, R₁₁, R₁₂, and R₁₃ is independently selected from H, methyl, alkyl, and heteroalkyl; or
R₁₀ and R₁₂, together with the atom to which they are attached, form a 4- to 6-membered cyclic or heterocyclic ring; or
R₁₁ and R₁₃, together with the atom to which they are attached, form a 4- to 6-membered cyclic or heterocyclic ring;
Z is selected from O, S, SO, SO₂, CRR′, CNRR′, COR′, and NR′, wherein each R and R′ is as defined above;
Y is selected from COR₂, SO₂R₂, (C═O)NR₂, (C═NH)R₂, (C═O)OR₂ and C₆H₅R₂, wherein R₂ is as defined above;
or a pharmaceutically acceptable salt, hydrate, tautomer, or optical isomer thereof.

7. A compound of formula (IB): 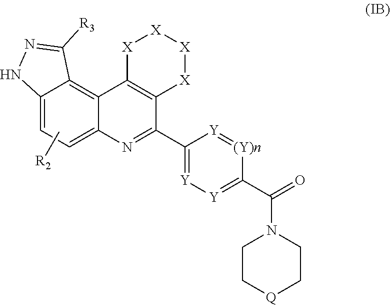
wherein
R₂ is selected from H, alkyl, heteroalkyl, aryl, heteroaryl, alkenyl, alkynyl, and halogen;
R₃ is selected from H, alkyl, heteroalkyl, aryl, heteroaryl, alkenyl, and alkynyl;
each X is independently selected from O, S, SO, SO₂, CRR′, CNRR′, COR′, NR′ and Xₙ, wherein n is 0-2; wherein each R and R′ is independently selected from H, halogen, CF₃, CN, alkyl, aryl, heteroalkyl, heteroaryl, CONR₂R₂, COR₂, SO₂R₂, (C═O)NR₂, (C═NH)R₂, (C═O)OR₂, C₆H₅R₂, morpholine and piperazine, wherein R₂ is as defined above; or
R and R′, together with the atom to which they are attached, form a 4- to 6-membered cyclic or heterocyclic ring;
Y is N or CR₂, wherein R₂ is as defined above;
n is 0 or 1;
Q is selected from NR″, CR₂R₂, N(COR₂), N(SO₂)R₂, N(C═O)NR₂, N(C═NH)R₂, and N(C═O)OR₂, with the proviso that when Q is NR″, R″ is not H, methyl, isopropyl, CH₂CH₂OH, COCH₃ or SO₂-methyl; and when Q is CR₂R₂, R₂ is not H or dimethyl amine;
or a pharmaceutically acceptable salt, hydrate, tautomer, or optical isomer thereof.

8. The compound of claim 7, wherein the compound of formula (IB) is 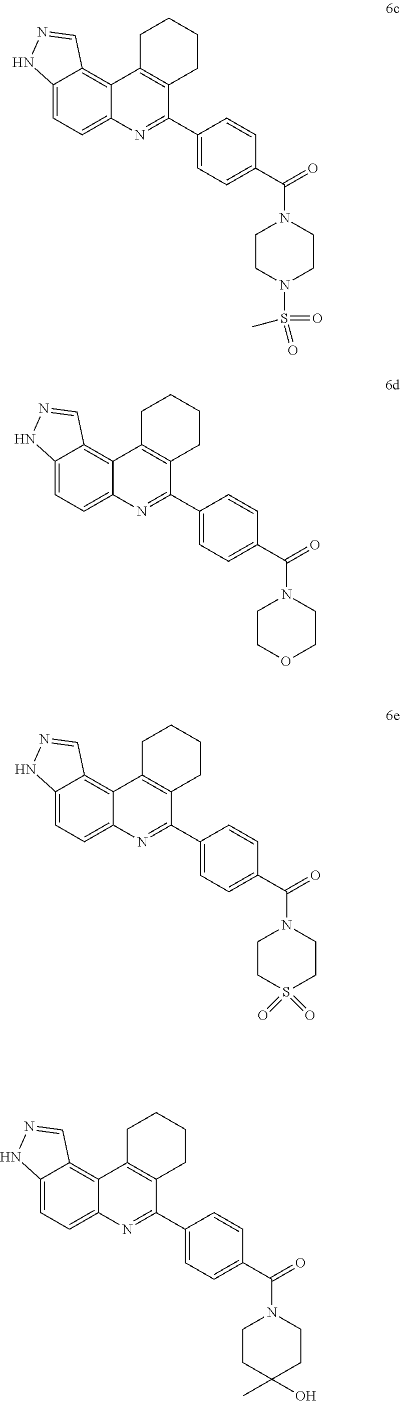 [SMILES: CS(=O)(=O)N1CCN(C(=O)c2ccc(-c3nc4ccc5[nH]ncc5c4c4c3CCCC4)cc2)CC1 ; O=C(c1ccc(-c2nc3ccc4[nH]ncc4c3c3c2CCCC3)cc1)N1CCOCC1 ; O=C(c1ccc(-c2nc3ccc4[nH]ncc4c3c3c2CCCC3)cc1)N1CCS(=O)(=O)CC1 ; CC1(O)CCN(C(=O)c2ccc(-c3nc4ccc5[nH]ncc5c4c4c3CCCC4)cc2)CC1] 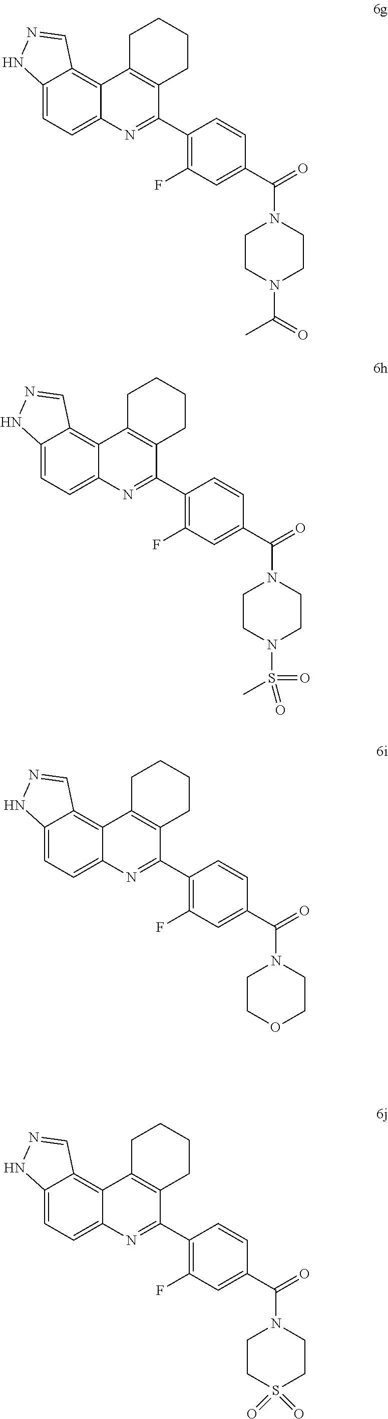 [SMILES: CC(=O)N1CCN(C(=O)c2ccc(-c3nc4ccc5[nH]ncc5c4c4c3CCCC4)c(F)c2)CC1 ; CS(=O)(=O)N1CCN(C(=O)c2ccc(-c3nc4ccc5[nH]ncc5c4c4c3CCCC4)c(F)c2)CC1 ; O=C(c1ccc(-c2nc3ccc4[nH]ncc4c3c3c2CCCC3)c(F)c1)N1CCOCC1 ; O=C(c1ccc(-c2nc3ccc4[nH]ncc4c3c3c2CCCC3)c(F)c1)N1CCS(=O)(=O)CC1] 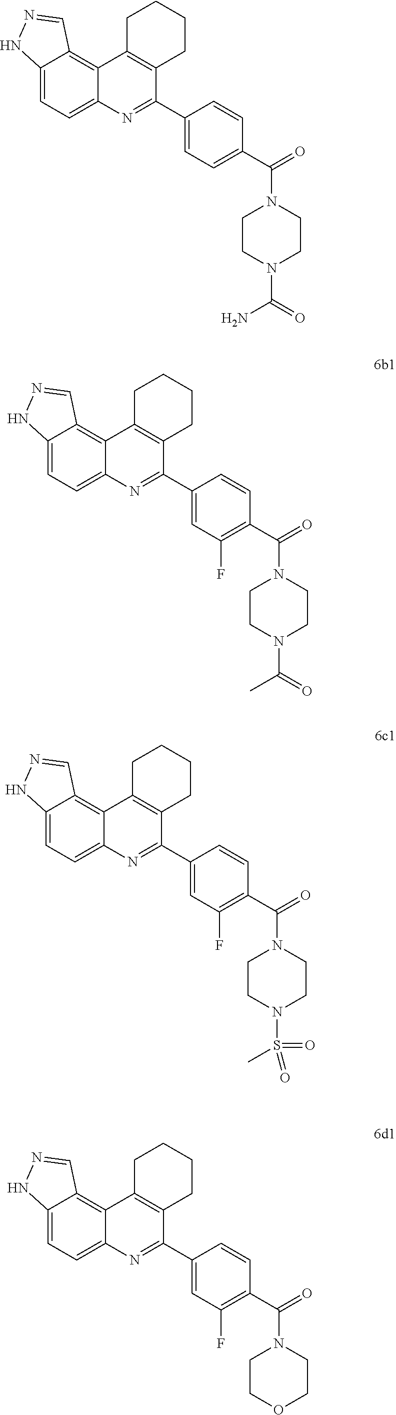 [SMILES: NC(=O)N1CCN(C(=O)c2ccc(-c3nc4ccc5[nH]ncc5c4c4c3CCCC4)cc2)CC1 ; CC(=O)N1CCN(C(=O)c2ccc(-c3nc4ccc5[nH]ncc5c4c4c3CCCC4)cc2F)CC1 ; CS(=O)(=O)N1CCN(C(=O)c2ccc(-c3nc4ccc5[nH]ncc5c4c4c3CCCC4)cc2F)CC1 ; O=C(c1ccc(-c2nc3ccc4[nH]ncc4c3c3c2CCCC3)cc1F)N1CCOCC1] 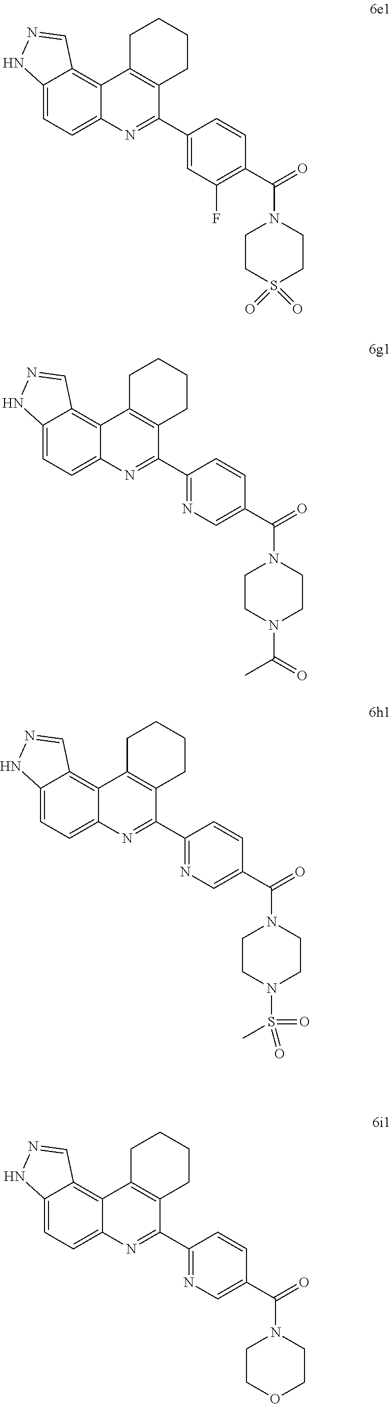 [SMILES: O=C(c1ccc(-c2nc3ccc4[nH]ncc4c3c3c2CCCC3)cc1F)N1CCS(=O)(=O)CC1 ; CC(=O)N1CCN(C(=O)c2ccc(-c3nc4ccc5[nH]ncc5c4c4c3CCCC4)nc2)CC1 ; CS(=O)(=O)N1CCN(C(=O)c2ccc(-c3nc4ccc5[nH]ncc5c4c4c3CCCC4)nc2)CC1 ; O=C(c1ccc(-c2nc3ccc4[nH]ncc4c3c3c2CCCC3)nc1)N1CCOCC1] 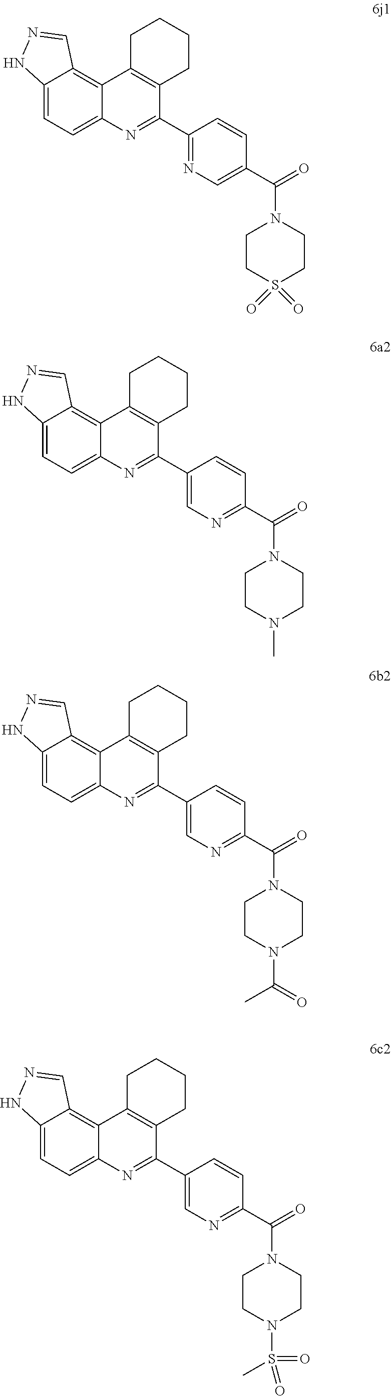 [SMILES: O=C(c1ccc(-c2nc3ccc4[nH]ncc4c3c3c2CCCC3)nc1)N1CCS(=O)(=O)CC1 ; CN1CCN(C(=O)c2ccc(-c3nc4ccc5[nH]ncc5c4c4c3CCCC4)cn2)CC1 ; CC(=O)N1CCN(C(=O)c2ccc(-c3nc4ccc5[nH]ncc5c4c4c3CCCC4)cn2)CC1 ; CS(=O)(=O)N1CCN(C(=O)c2ccc(-c3nc4ccc5[nH]ncc5c4c4c3CCCC4)cn2)CC1] 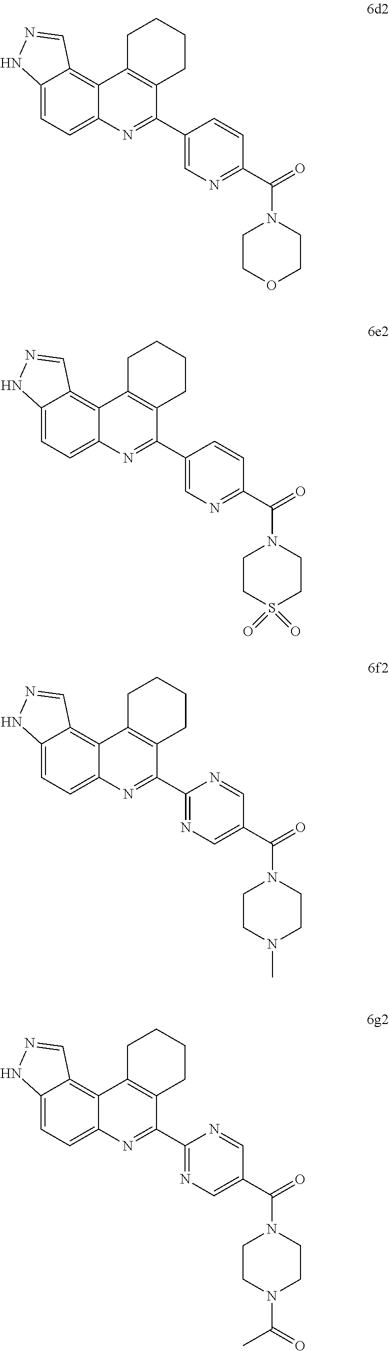 [SMILES: O=C(c1ccc(-c2nc3ccc4[nH]ncc4c3c3c2CCCC3)cn1)N1CCOCC1 ; O=C(c1ccc(-c2nc3ccc4[nH]ncc4c3c3c2CCCC3)cn1)N1CCS(=O)(=O)CC1 ; CN1CCN(C(=O)c2cnc(-c3nc4ccc5[nH]ncc5c4c4c3CCCC4)nc2)CC1 ; CC(=O)N1CCN(C(=O)c2cnc(-c3nc4ccc5[nH]ncc5c4c4c3CCCC4)nc2)CC1] 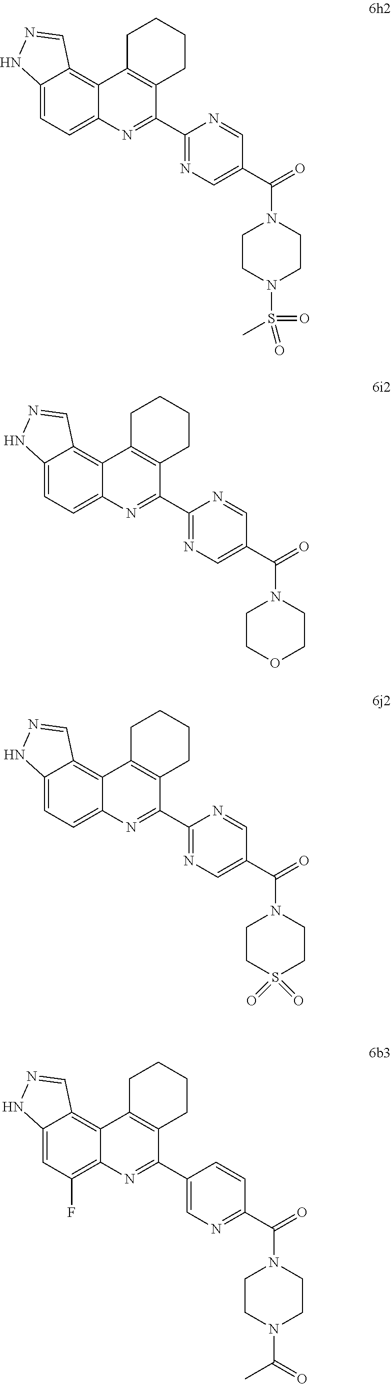 [SMILES: CS(=O)(=O)N1CCN(C(=O)c2cnc(-c3nc4ccc5[nH]ncc5c4c4c3CCCC4)nc2)CC1 ; O=C(c1cnc(-c2nc3ccc4[nH]ncc4c3c3c2CCCC3)nc1)N1CCOCC1 ; O=C(c1cnc(-c2nc3ccc4[nH]ncc4c3c3c2CCCC3)nc1)N1CCS(=O)(=O)CC1 ; CC(=O)N1CCN(C(=O)c2ccc(-c3nc4c(F)cc5[nH]ncc5c4c4c3CCCC4)cn2)CC1] 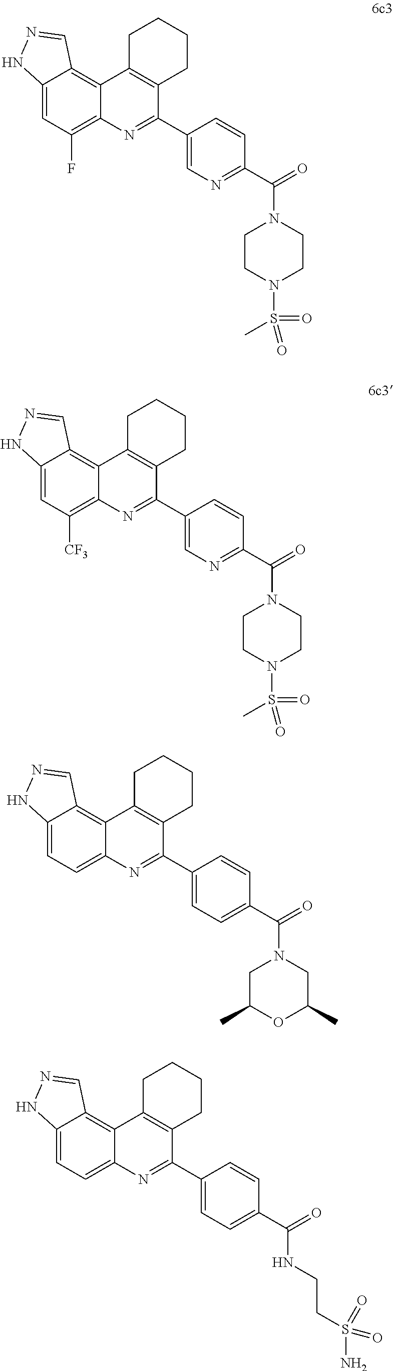 [SMILES: CS(=O)(=O)N1CCN(C(=O)c2ccc(-c3nc4c(F)cc5[nH]ncc5c4c4c3CCCC4)cn2)CC1 ; CS(=O)(=O)N1CCN(C(=O)c2ccc(-c3nc4c(C(F)(F)F)cc5[nH]ncc5c4c4c3CCCC4)cn2)CC1 ; C[C@@H]1CN(C(=O)c2ccc(-c3nc4ccc5[nH]ncc5c4c4c3CCCC4)cc2)C[C@H](C)O1 ; NS(=O)(=O)CCNC(=O)c1ccc(-c2nc3ccc4[nH]ncc4c3c3c2CCCC3)cc1] 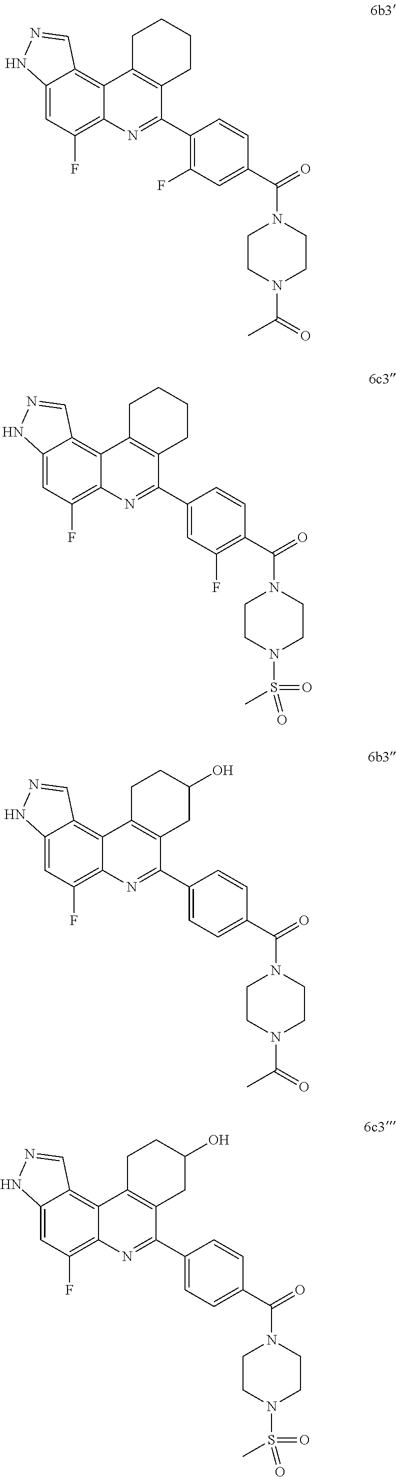 [SMILES: CC(=O)N1CCN(C(=O)c2ccc(-c3nc4c(F)cc5[nH]ncc5c4c4c3CCCC4)c(F)c2)CC1 ; CS(=O)(=O)N1CCN(C(=O)c2ccc(-c3nc4c(F)cc5[nH]ncc5c4c4c3CCCC4)cc2F)CC1 ; CC(=O)N1CCN(C(=O)c2ccc(-c3nc4c(F)cc5[nH]ncc5c4c4c3CC(O)CC4)cc2)CC1 ; CS(=O)(=O)N1CCN(C(=O)c2ccc(-c3nc4c(F)cc5[nH]ncc5c4c4c3CC(O)CC4)cc2)CC1] 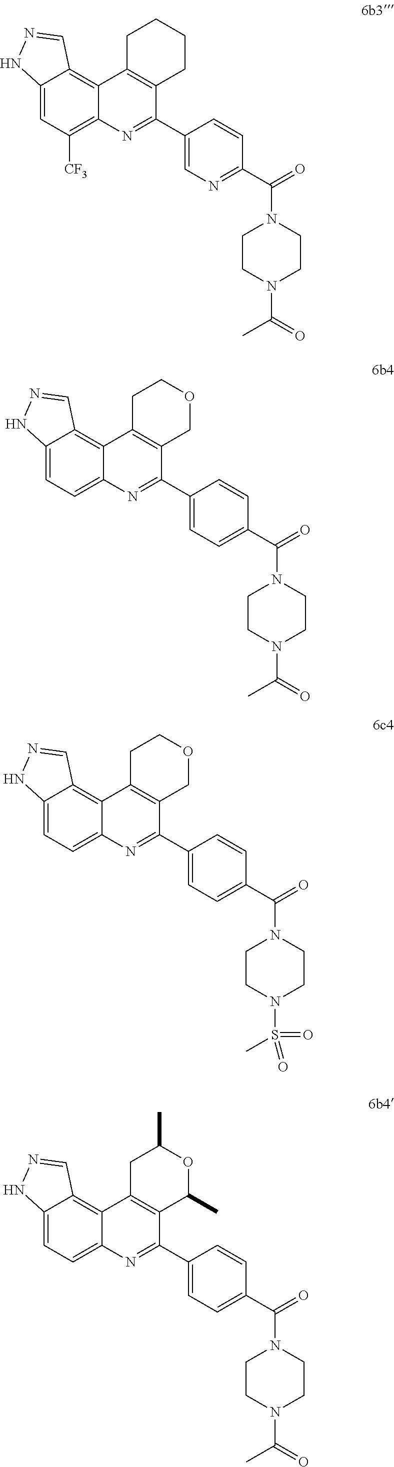 [SMILES: CC(=O)N1CCN(C(=O)c2ccc(-c3nc4c(C(F)(F)F)cc5[nH]ncc5c4c4c3CCCC4)cn2)CC1 ; CC(=O)N1CCN(C(=O)c2ccc(-c3nc4ccc5[nH]ncc5c4c4c3COCC4)cc2)CC1 ; CS(=O)(=O)N1CCN(C(=O)c2ccc(-c3nc4ccc5[nH]ncc5c4c4c3COCC4)cc2)CC1 ; CC(=O)N1CCN(C(=O)c2ccc(-c3nc4ccc5[nH]ncc5c4c4c3[C@H](C)O[C@H](C)C4)cc2)CC1] 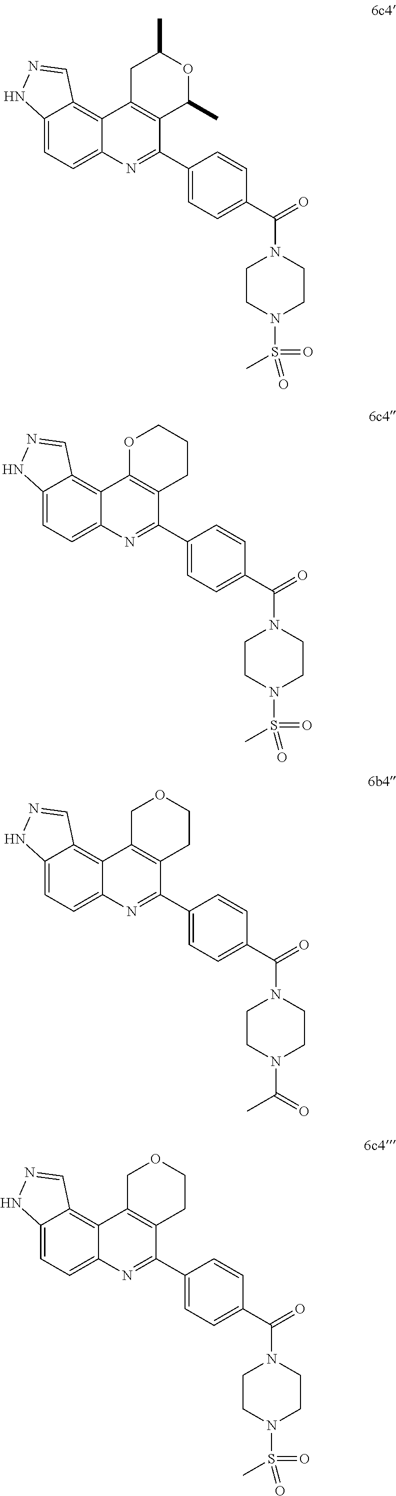 [SMILES: C[C@@H]1Cc2c(c(-c3ccc(C(=O)N4CCN(S(C)(=O)=O)CC4)cc3)nc3ccc4[nH]ncc4c23)[C@H](C)O1 ; CS(=O)(=O)N1CCN(C(=O)c2ccc(-c3nc4ccc5[nH]ncc5c4c4c3CCCO4)cc2)CC1 ; CC(=O)N1CCN(C(=O)c2ccc(-c3nc4ccc5[nH]ncc5c4c4c3CCOC4)cc2)CC1 ; CS(=O)(=O)N1CCN(C(=O)c2ccc(-c3nc4ccc5[nH]ncc5c4c4c3CCOC4)cc2)CC1] 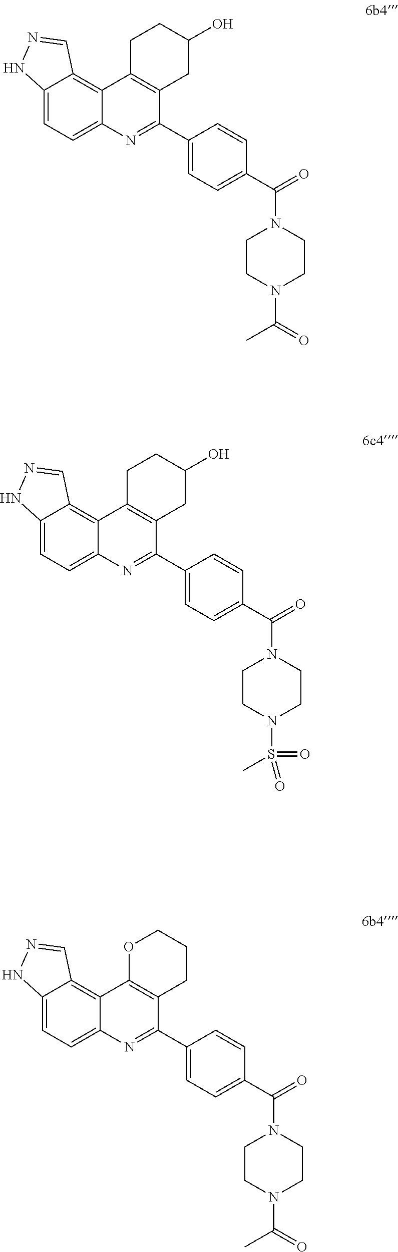 [SMILES: CC(=O)N1CCN(C(=O)c2ccc(-c3nc4ccc5[nH]ncc5c4c4c3CC(O)CC4)cc2)CC1 ; CS(=O)(=O)N1CCN(C(=O)c2ccc(-c3nc4ccc5[nH]ncc5c4c4c3CC(O)CC4)cc2)CC1 ; CC(=O)N1CCN(C(=O)c2ccc(-c3nc4ccc5[nH]ncc5c4c4c3CCCO4)cc2)CC1] 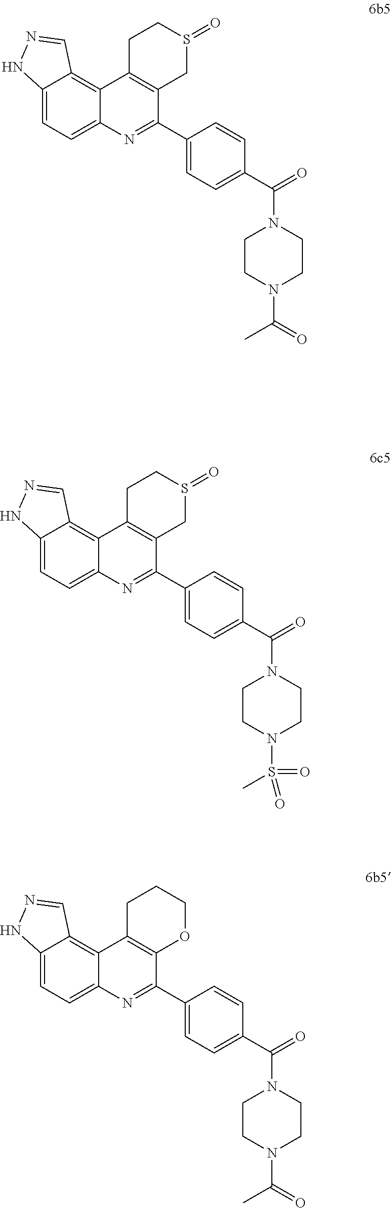 [SMILES: CC(=O)N1CCN(C(=O)c2ccc(-c3nc4ccc5[nH]ncc5c4c4c3CS(=O)CC4)cc2)CC1 ; CS(=O)(=O)N1CCN(C(=O)c2ccc(-c3nc4ccc5[nH]ncc5c4c4c3CS(=O)CC4)cc2)CC1 ; CC(=O)N1CCN(C(=O)c2ccc(-c3nc4ccc5[nH]ncc5c4c4c3OCCC4)cc2)CC1] 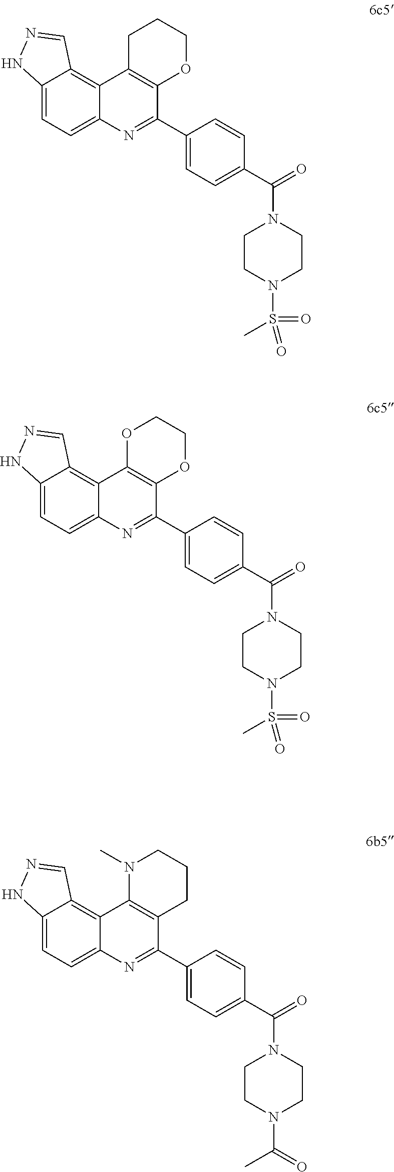 [SMILES: CS(=O)(=O)N1CCN(C(=O)c2ccc(-c3nc4ccc5[nH]ncc5c4c4c3OCCC4)cc2)CC1 ; CS(=O)(=O)N1CCN(C(=O)c2ccc(-c3nc4ccc5[nH]ncc5c4c4c3OCCO4)cc2)CC1 ; CC(=O)N1CCN(C(=O)c2ccc(-c3nc4ccc5[nH]ncc5c4c4c3CCCN4C)cc2)CC1] 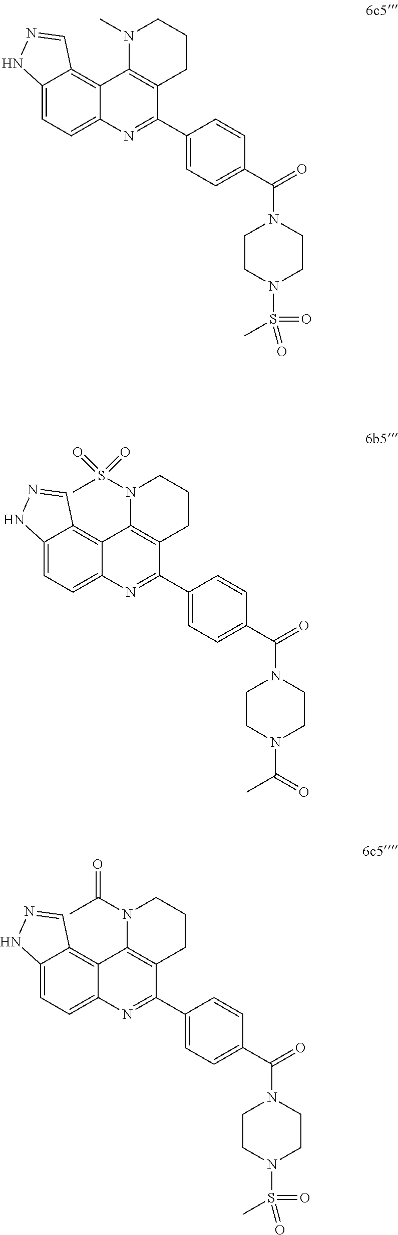 [SMILES: CN1CCCc2c(-c3ccc(C(=O)N4CCN(S(C)(=O)=O)CC4)cc3)nc3ccc4[nH]ncc4c3c21 ; CC(=O)N1CCN(C(=O)c2ccc(-c3nc4ccc5[nH]ncc5c4c4c3CCCN4S(C)(=O)=O)cc2)CC1 ; CC(=O)N1CCCc2c(-c3ccc(C(=O)N4CCN(S(C)(=O)=O)CC4)cc3)nc3ccc4[nH]ncc4c3c21] 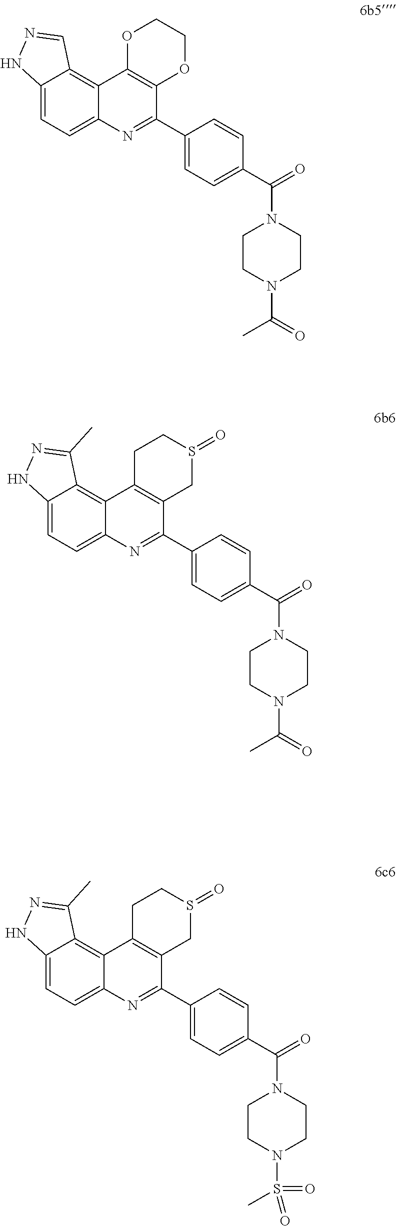 [SMILES: CC(=O)N1CCN(C(=O)c2ccc(-c3nc4ccc5[nH]ncc5c4c4c3OCCO4)cc2)CC1 ; CC(=O)N1CCN(C(=O)c2ccc(-c3nc4ccc5[nH]nc(C)c5c4c4c3CS(=O)CC4)cc2)CC1 ; Cc1n[nH]c2ccc3nc(-c4ccc(C(=O)N5CCN(S(C)(=O)=O)CC5)cc4)c4c(c3c12)CCS(=O)C4] 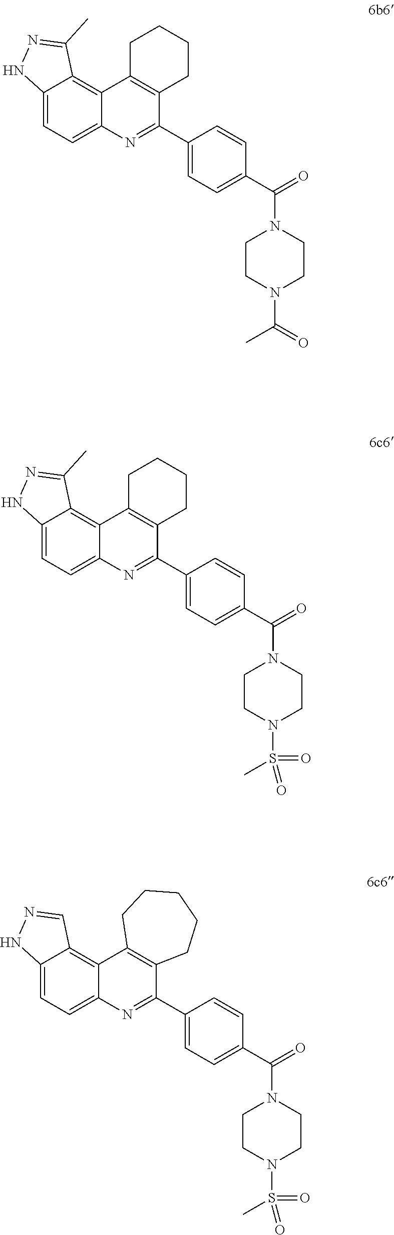 [SMILES: CC(=O)N1CCN(C(=O)c2ccc(-c3nc4ccc5[nH]nc(C)c5c4c4c3CCCC4)cc2)CC1 ; Cc1n[nH]c2ccc3nc(-c4ccc(C(=O)N5CCN(S(C)(=O)=O)CC5)cc4)c4c(c3c12)CCCC4 ; CS(=O)(=O)N1CCN(C(=O)c2ccc(-c3nc4ccc5[nH]ncc5c4c4c3CCCCC4)cc2)CC1] 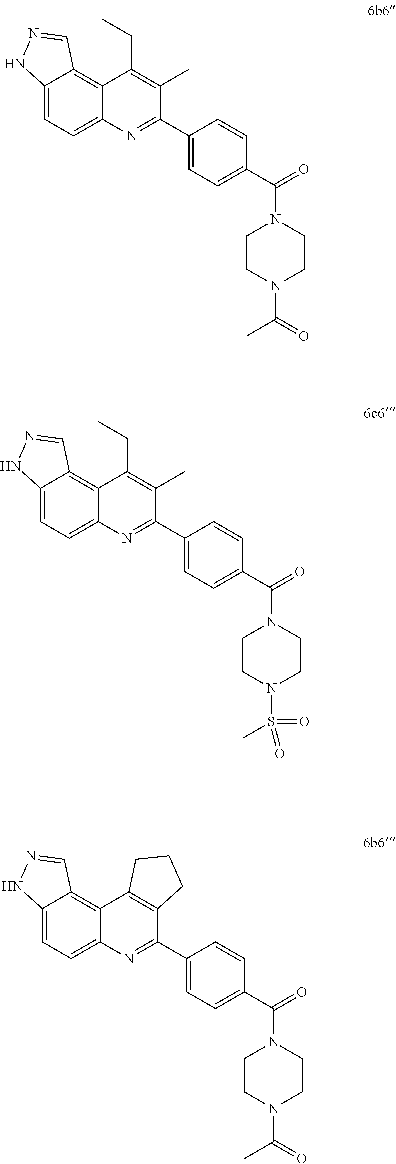 [SMILES: CCc1c(C)c(-c2ccc(C(=O)N3CCN(C(C)=O)CC3)cc2)nc2ccc3[nH]ncc3c12 ; CCc1c(C)c(-c2ccc(C(=O)N3CCN(S(C)(=O)=O)CC3)cc2)nc2ccc3[nH]ncc3c12 ; CC(=O)N1CCN(C(=O)c2ccc(-c3nc4ccc5[nH]ncc5c4c4c3CCC4)cc2)CC1] 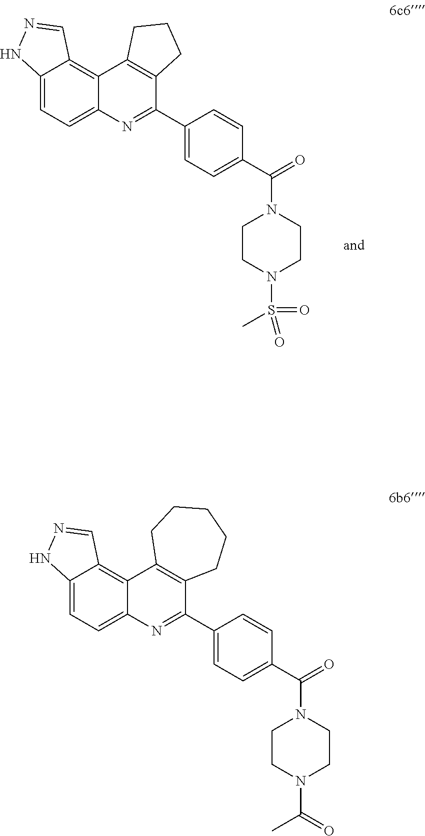 [SMILES: CS(=O)(=O)N1CCN(C(=O)c2ccc(-c3nc4ccc5[nH]ncc5c4c4c3CCC4)cc2)CC1 ; CC(=O)N1CCN(C(=O)c2ccc(-c3nc4ccc5[nH]ncc5c4c4c3CCCCC4)cc2)CC1]
or a pharmaceutically acceptable salt, hydrate, tautomer, or optical isomer thereof.

9. A compound of formula (I) represented by structures: 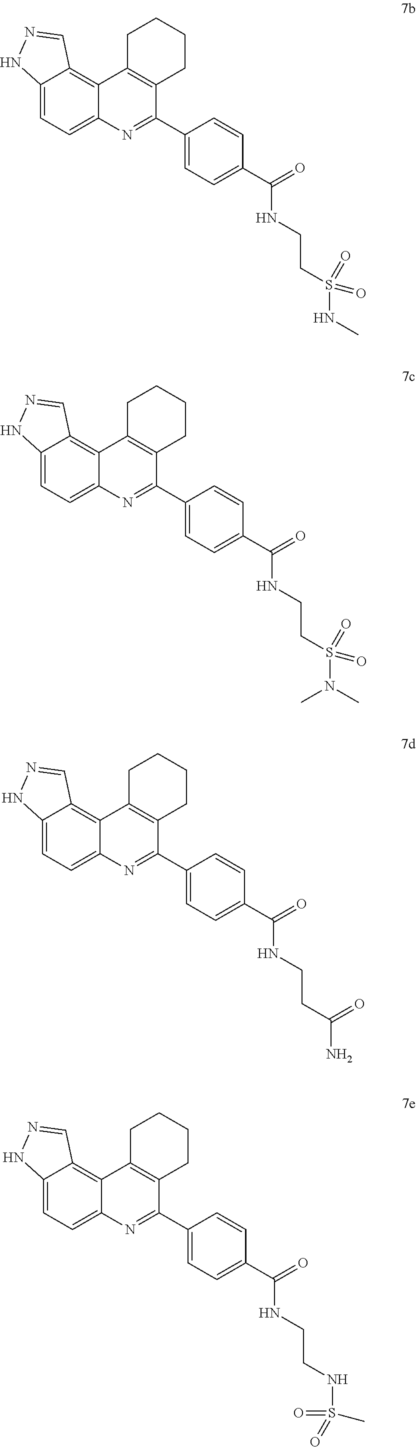 [SMILES: CNS(=O)(=O)CCNC(=O)c1ccc(-c2nc3ccc4[nH]ncc4c3c3c2CCCC3)cc1 ; CN(C)S(=O)(=O)CCNC(=O)c1ccc(-c2nc3ccc4[nH]ncc4c3c3c2CCCC3)cc1 ; NC(=O)CCNC(=O)c1ccc(-c2nc3ccc4[nH]ncc4c3c3c2CCCC3)cc1 ; CS(=O)(=O)NCCNC(=O)c1ccc(-c2nc3ccc4[nH]ncc4c3c3c2CCCC3)cc1] 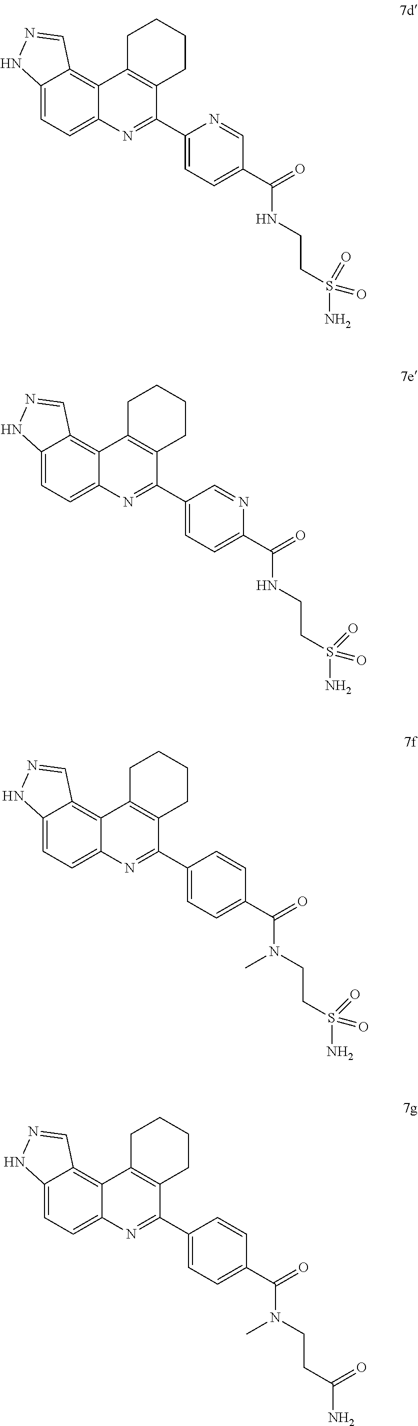 [SMILES: NS(=O)(=O)CCNC(=O)c1ccc(-c2nc3ccc4[nH]ncc4c3c3c2CCCC3)nc1 ; NS(=O)(=O)CCNC(=O)c1ccc(-c2nc3ccc4[nH]ncc4c3c3c2CCCC3)cn1 ; CN(CCS(N)(=O)=O)C(=O)c1ccc(-c2nc3ccc4[nH]ncc4c3c3c2CCCC3)cc1 ; CN(CCC(N)=O)C(=O)c1ccc(-c2nc3ccc4[nH]ncc4c3c3c2CCCC3)cc1] 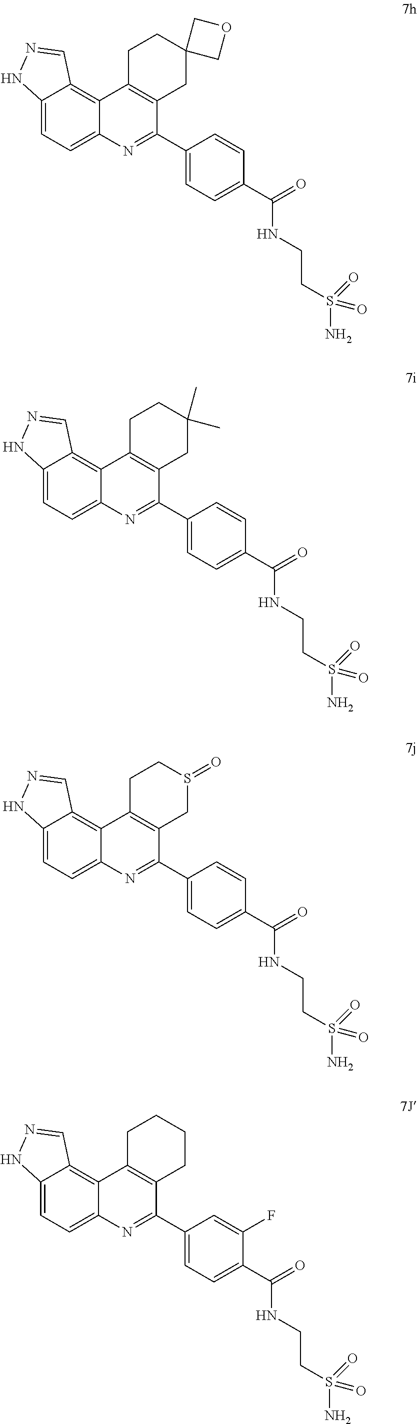 [SMILES: NS(=O)(=O)CCNC(=O)c1ccc(-c2nc3ccc4[nH]ncc4c3c3c2CC2(CC3)COC2)cc1 ; CC1(C)CCc2c(c(-c3ccc(C(=O)NCCS(N)(=O)=O)cc3)nc3ccc4[nH]ncc4c23)C1 ; NS(=O)(=O)CCNC(=O)c1ccc(-c2nc3ccc4[nH]ncc4c3c3c2CS(=O)CC3)cc1 ; NS(=O)(=O)CCNC(=O)c1ccc(-c2nc3ccc4[nH]ncc4c3c3c2CCCC3)cc1F] 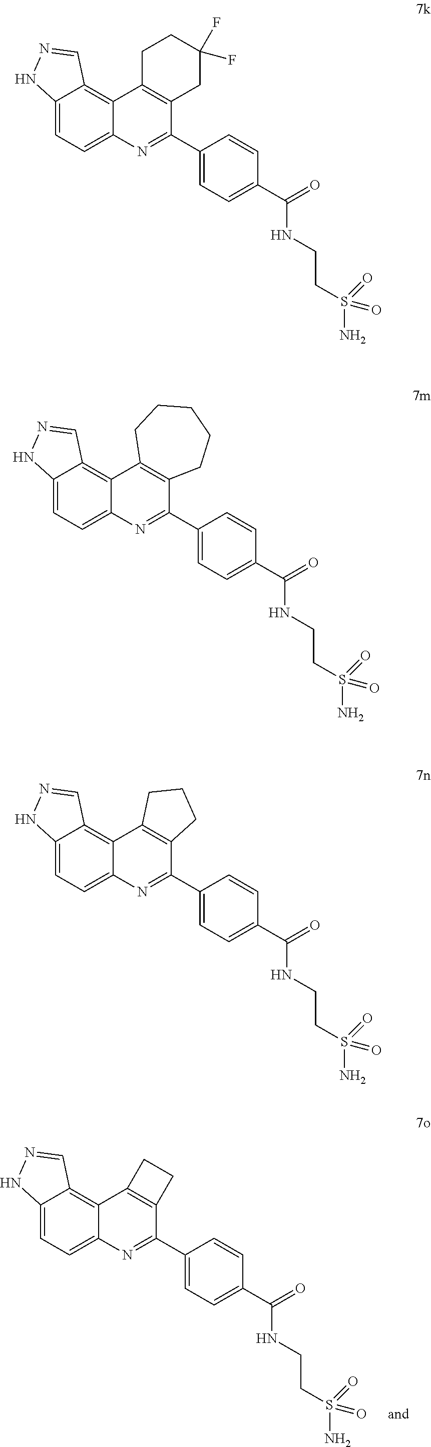 [SMILES: NS(=O)(=O)CCNC(=O)c1ccc(-c2nc3ccc4[nH]ncc4c3c3c2CC(F)(F)CC3)cc1 ; NS(=O)(=O)CCNC(=O)c1ccc(-c2nc3ccc4[nH]ncc4c3c3c2CCCCC3)cc1 ; NS(=O)(=O)CCNC(=O)c1ccc(-c2nc3ccc4[nH]ncc4c3c3c2CCC3)cc1 ; NS(=O)(=O)CCNC(=O)c1ccc(-c2nc3ccc4[nH]ncc4c3c3c2CC3)cc1] 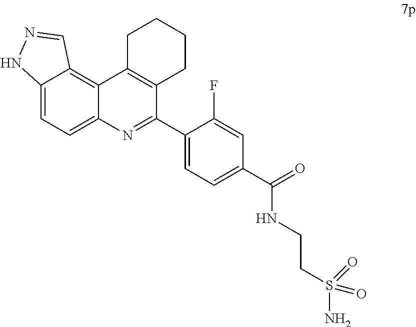 [SMILES: NS(=O)(=O)CCNC(=O)c1ccc(-c2nc3ccc4[nH]ncc4c3c3c2CCCC3)c(F)c1]
or a pharmaceutically acceptable salt, hydrate, tautomer, or optical isomer thereof.

10. A pharmaceutical composition comprising a compound of formula (I) of claim 9, or a pharmaceutically acceptable salt, hydrate, tautomer, or optical isomer thereof and a pharmaceutically acceptable carrier, excipient, or diluent.

11. A pharmaceutical composition comprising a compound of formula (IA) of claim 4 or a pharmaceutically acceptable salt, hydrate, tautomer, or optical isomer thereof, and a pharmaceutically acceptable carrier, excipient, or diluent.

12. A pharmaceutical composition comprising a compound of formula (IB) of claim 7 or a pharmaceutically acceptable salt, hydrate, tautomer, or optical isomer thereof, and a pharmaceutically acceptable carrier, excipient, or diluent.

13. A method of treating or inhibiting a disease associated with overacting of Stimulator of Interferon Genes (STING) comprising administering to a patient a therapeutically effective amount of a compound of formula (IA) of claim 4 or a pharmaceutical composition comprising the compound and a pharmaceutically acceptable carrier, excipient, or diluent.

14. The method of claim 13, wherein the disease associated with overacting of STING is selected from inflammatory diseases, autoimmune diseases, diabetes, cancer, traumatic brain injury, and fibrosis.

15. The method of claim 14, wherein the disease associated with overacting of STING is the inflammatory disease.

16. A method of treating or inhibiting a disease associated with overacting of Stimulator of Interferon Genes (STING) comprising administering to a patient a therapeutically effective amount of a compound of formula (IB) of claim 7 or a pharmaceutical composition comprising the compound and a pharmaceutically acceptable carrier, excipient, or diluent.

17. The method of claim 16, wherein the disease associated with overacting of STING is selected from inflammatory diseases, autoimmune diseases, diabetes, cancer, traumatic brain injury, and fibrosis.

18. The method of claim 17, wherein the disease associated with overacting of STING is the inflammatory disease.
